(19) United States
(12) Patent Application Publication
(10) Pub. No.: US 2026/0193694 A1
(43) Pub. Date: Jul. 9, 2026

(54) OPTICAL GENETIC SCREENS OF INTRACELLULAR AND INTERCELLULAR TRANSCRIPTIONAL CIRCUITS WITH PERTURB-FISH

(71) Applicants: The Broad Institute, Inc., Cambridge, MA (US); Trustees of Boston University, Boston, MA (US)

(72) Inventors: Samouil Farhi, Cambridge, MA (US); Loic Binan, Cambridge, MA (US); Brian Cleary, Boston, MA (US)

(21) Appl. No.: 19/553,095

(22) Filed: Feb. 27, 2026

Related U.S. Application Data
(63) Continuation of application No. PCT/US2024/044496, filed on Aug. 29, 2024.
(60) Provisional application No. 63/535,281, filed on Aug. 29, 2023.

(51) Int. Cl. C12Q 1/6841

(52) U.S. Cl. CPC C12Q 1/6841

(57) ABSTRACT

The subject matter disclosed herein is generally directed to methods for highly multiplexed spatially resolved optical perturbation screening and kits thereof. The methods use sequence specific perturbations that can be amplified in situ and optically decoded. The perturbations can be paired with optically decoded gene expression data.

### CROSS-REFERENCE TO RELATED APPLICATIONS

This application is a continuation application of International Patent Application number PCT/US2024/044496, filed Aug. 29, 2024, which claims the benefit of U.S. Provisional Application No. 63/535,281, filed Aug. 29, 2023. The entire contents of the above-identified applications are hereby fully incorporated herein by reference.

### STATEMENT REGARDING FEDERALLY SPONSORED RESEARCH

This invention was made with government support under Grant Nos. MH128366 and NM121289 awarded by the National Institutes of Health. The government has certain rights in the invention.

### REFERENCE TO AN ELECTRONIC SEQUENCE LISTING

The contents of the electronic sequence listing (“BROD-5905US_ST26.xml; Size is 55,504 bytes and it was created on Feb. 10, 2026) is herein incorporated by reference in its entirety.

### TECHNICAL FIELD

The subject matter disclosed herein is generally directed to methods for highly multiplexed spatially resolved optical perturbation screening and kits thereof.

### BACKGROUND

Perturb-seq allows perturbing a gene using CRISPR and recording the effect on the transcriptome. Perturb-seq uses a microfluidics approach for sequencing, and therefore does not allow keeping spatial information in the data (see, e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Jaitin D A, Weiner A, Yofe I, et al. Dissecting Immune Circuits by Linking CRISPR-Pooled Screens with Single-Cell RNA-Seq. Cell. 2016; 167(7):1883-1896.e15; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; Replogle, et al., “Combinatorial single-cell CRISPR screens by direct guide RNA capture and targeted sequencing” Nat Biotechnol (2020). doi.org/10.1038/s41587-020-0470-y; Schraivogel D, Gschwind A R, Milbank J H, et al. “Targeted Perturb-seq enables genome-scale genetic screens in single cells”. Nat Methods. 2020; 17(6):629-635; Frangieh C J, Melms J C, Thakore P I, et al. Multimodal pooled Perturb-CITE-seq screens in patient models define mechanisms of cancer immune evasion. Nat Genet. 2021; 53(3):332-341; US patent application publication number US20200283843A1; and U.S. Pat. No. 11,214,797B2). Prior technologies for optical spatial recording of transcriptomes cannot detect barcodes identifying a perturbation (see, e.g., Moffitt J R, Hao J, Wang G, Chen K H, Babcock H P, Zhuang X. High-throughput single-cell gene-expression profiling with multiplexed error-robust fluorescence in situ hybridization. Proc Natl Acad Sci USA. 2016; 113(39):11046-11051). Additionally, technologies for recording a DNA barcode in a cell cannot be used for multiplexing and is not compatible with MERFISH (see, e.g., Askary A, Sanchez-Guardado L, Linton J M, et al. In situ readout of DNA barcodes and single base edits facilitated by in vitro transcription [published correction appears in Nat Biotechnol. 2020 February; 38(2):245]. Nat Biotechnol. 2020; 38(1):66-75).

Citation or identification of any document in this application is not an admission that such a document is available as prior art to the present invention.

### SUMMARY

In one aspect, the present invention provides for a method for perturbation screening with spatially resolved readouts comprising: (a) perturbing a plurality of cells by introducing one or more perturbation constructs to the plurality of cells, each perturbation construct encoding for one or more sequence specific perturbations, wherein the one or more sequence specific perturbations comprise a perturbation sequence identifying the perturbation, said sequence operably linked to at least two promoters comprising a Pol III promoter and a phage promoter, and wherein the plurality of cells maintains a spatial localization; (b) fixing the perturbed cells, whereby the spatial localization of the plurality of cells is fixed; (c) contacting the fixed cells with one or more phage polymerases and reagents for in vitro transcription of the perturbation sequences; (d) encoding the plurality of cells by contacting the plurality of cells with: (i) encoding probes specific for a perturbation sequence, each probe comprising a targeting sequence specific for one perturbation sequence and one or more readout sequences specific for each perturbation; and (ii) encoding probes specific for mRNAs expressed in the plurality of cells, each probe comprising a targeting sequence specific for an mRNA sequence and one or more readout sequences specific for each mRNA sequence; (e) contacting the fixed and encoded plurality of cells with fluorescently labeled readout probes specific for a readout sequence and acquiring spatially resolved images for each readout probe using a microscope; and (f) decoding the perturbation and mRNA expression for each cell in the plurality of cells based on the images acquired. In certain embodiments, the one or more perturbation constructs comprise one or more anchor sequences downstream of the perturbation sequence, wherein the one or more anchor sequences are the same for every perturbation construct.

In certain embodiments, the encoding step (d) further comprises contacting the plurality of cells with acrydite-modified anchor probes comprising a sequence specific to an anchor sequence and contacting the plurality of cells with acrydite-modified anchor probes comprising a poly(dT) sequence, wherein the method further comprises embedding the fixed cells in a polymerized hydrogel; and removing cell components not linked to the hydrogel through an anchor probe. In certain embodiments, the polymerized gel is a non-swellable hydrogel, optionally, a polyacrylamide hydrogel. In certain embodiments, the polymerized gel is a swellable hydrogel, optionally, a polyacrylate hydrogel. In certain embodiments, the anchor probes comprise locked nucleic acids (LNAs).

In certain embodiments, the plurality of cells is grown on a solid support to maintain a spatial localization. In certain embodiments, the solid support is a glass slide or coverslip. In certain embodiments, the plurality of cells is a tissue explant. In certain embodiments, the plurality of cells is a tissue, wherein steps (a) and (b) comprise perturbing the plurality of cells in vivo and fixing the perturbed tissue to a slide.

In certain embodiments, the one or more sequence specific perturbations comprises a CRISPR system. In certain embodiments, the perturbation sequence is a guide sequence. In certain embodiments, the one or more sequence specific perturbations is an RNAi or antisense system. In certain embodiments, the perturbation sequence is an RNAi or antisense sequence.

In certain embodiments, the readout sequences of the encoding probes for encoding the perturbation sequences are different from the readout sequences of the encoding probes for encoding the mRNA sequences, and wherein step (f) is performed in two steps, one step for perturbations and one step for mRNA.

In certain embodiments, the one or more perturbation constructs are integrated into the genome of the perturbed cells. In certain embodiments, the one or more perturbation constructs are introduced by a viral vector, optionally, a lentiviral vector. In certain embodiments, the one or more perturbation constructs are introduced at a multiplicity of infection (MOI) where each cell in the plurality of cells receives one or zero perturbation constructs.

In certain embodiments, the cells are grown at high density greater than 3,000 cells/cm², 4,000 cells/cm², or 5,000 cells/cm²; or about 10⁷ cells/mL; or about 90-100% confluence. In certain embodiments, the cells are grown at low density less than 50 cells/cm², 100 cells/cm², or 200 cells/cm²; or about 10⁵ cells/mL or 10⁴ cells/mL; or about 50% confluence.

In certain embodiments, the method further comprises linking the perturbation and mRNA expression to one or more additional phenotypes detectable by microscopy. In certain embodiments, the one or more additional phenotypes comprise cell morphology or biomolecule organization. In certain embodiments, the one or more additional phenotypes comprise any time resolved phenotype, optionally, calcium imaging, voltage imaging, dynamic metabolite measurements, markers of cell stress, and/or cell migration. In certain embodiments, after step (a), perturbation, and before step (b), fixing, a movie of the plurality of cells is recorded. In certain embodiments, live cell markers are recorded.

In another aspect, the present invention provides for a kit comprising a library of perturbation constructs, each perturbation construct encoding for one or more sequence specific perturbations, wherein the one or more sequence specific perturbations comprise a perturbation sequence identifying the perturbation, said sequence operably linked to at least two promoters comprising a Pol III promoter and a phage promoter. In certain embodiments, the one or more sequence specific perturbations comprises a CRISPR system. In certain embodiments, the perturbation sequence is a guide sequence. In certain embodiments, each perturbation construct is a viral vector, optionally, a lentiviral vector. In certain embodiments, the kit further comprises encoding probes specific for a perturbation sequence, each probe comprising a targeting sequence specific for one perturbation sequence and one or more readout sequences specific for each perturbation. In certain embodiments, the kit further comprises encoding probes specific for mRNA sequences, each probe comprising a targeting sequence specific for an mRNA sequence and one or more readout sequences specific for each mRNA sequence. In certain embodiments, the perturbation constructs comprise one or more anchor sequences downstream of the perturbation sequence, wherein the one or more anchor sequences are the same for every perturbation construct. In certain embodiments, the kit further comprises acrydite-modified anchor probes comprising a sequence specific to the one or more anchor sequences downstream of the perturbation sequence. In certain embodiments, the kit further comprises acrydite-modified anchor probes comprising a poly(dT) sequence. In certain embodiments, the anchor probes comprise locked nucleic acids (LNAs). In certain embodiments, the kit further comprises fluorescently labeled readout probes specific for a readout sequence on the encoding probes.

These and other aspects, objects, features, and advantages of the example embodiments will become apparent to those having ordinary skill in the art upon consideration of the following detailed description of example embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention may be utilized, and the accompanying drawings of which:

FIG. 1—Exemplary perturbation construct. The perturbation construct inserted into the DNA of a target cell. The guide RNA is in vitro transcribed in fixed cells. Encoding probes hybridize to the transcribed guide RNAs to allow optical identification of the perturbation.

FIG. 2A-FIG. 2B—In vitro transcription amplification allows identification of guides in astrocytes. RNA identity is encoded across 15 images. Each gRNA used in the experiment is expected to be “on” in 4 out of 15 images. Each barcode is different from any other barcode by at least 4 bits (hamming weight 4 hamming distance 4, as was used in MERFISH), allowing error correction (i.e., if a spot appears “on” 5 times, it can be corrected and the identity decoded). FIG. 2A. Shows 15 images for a single cell. FIG. 2B. Shows a single image for a field of cells.

FIG. 3—Example image in THP1 cells. Shown are Cell mask in blue, mRNA transcripts (identified with MERFISH) in green, and guide RNAs in red.

FIG. 4—Example experimental timeline. Protocol showing days 1-15.

FIG. 5—Distributions of guides during perturb-FISH experiment. (top) shows the distribution of guides as they were in the solution of DNA used to make viruses. (middle) shows the distribution of guides as decoded from images. (bottom) shows the variation in guides cloned in library and decoded.

FIG. 6—Clustered effect from gRNA (y-axis) on gene expression (x-axis) in THP1 cells. Heat map showing perturbed genes (35 gRNAs) on the y-axis, decoded through the T7 amplification and encoding/decoding, and recorded genes (mRNA) on the x-axis decoded with MERFISH.

FIG. 7—Comparison of perturbation effects measured with perturb-SEQ to effects measured with perturb-FISH. Heatmap showing perturbation gene expression effects in THP1 cells. gRNAs on the y-axis and gene expression on x-axis. Perturb-FISH (lower left of each square) and perturb-seq (upper right of each square).

FIG. 8A-FIG. 8D—Individual gene comparison of perturbation effects measured with perturb-SEQ to effects measured with perturb-FISH. In these plots, one spot represents a gene, evaluated with perturb-FISH (y axis) or perturb-seq (x axis). FIG. 8A. The effect of blocking IRAK1 on gene expression. FIG. 8B. The effect of blocking MYD88 on gene expression. FIG. 8C. The effect of blocking NFKB1 on gene expression. FIG. 8D. The effect of blocking RELA on gene expression.

FIG. 9—Comparison of perturbation effects measured with perturb-FISH in high density (lower left) vs. low density (upper right) cells. Heatmap showing the effect of perturbing the genes on y-axis on the genes on x-axis. Each square shows that effect in high density cells (bottom left) and low-density cells (top right), revealing variations with cell density.

FIG. 10A-FIG. 10H—Exemplary perturb-FISH experiment to identify gene networks in the macrophage response to LPS stimulation. FIG. 10A. Package virus encoding a guide RNA and infect cells. FIG. 10B. Select cells that received a guide RNA. Live cells express guide RNAs from the U6 promoter. FIG. 10C. Optional step where cells can be differentiated and/or stimulated. FIG. 10D. The cells are fixed and the guide sequences are amplified by in vitro transcription with T7 RNA polymerase from the T7 promoter. FIG. 10E. Optional post fixation with paraformaldehyde (PFA). The amplified guide RNA sequences and mRNA sequences are encoded with encoding probes and stained with anchoring probes. The fixed cells are embedded in a polymerized gel, such that the anchor probes are anchored in the gel. The gel is cleared of lipids, proteins and any components not anchored to the gel. FIG. 10F. Images are recorded for each readout probe for guide RNAs. Each encoding probe includes four readout sequences that each have one complimentary readout probe. The encoding probes for the guide RNAs contain 4 out of 15 readout sequences. An image is generated for each of the 15 readout probes. Each guide RNA is expected to have a signal for 4 of the 15 readout probes. FIG. 10G. Images are recorded for each readout probe for mRNAs. Each mRNA encoding probe includes four readout sequences that each have one readout probe. Each mRNA is bound by multiple encoding probes. The encoding probes for mRNAs contain 4 out of 16 readout sequences. An image is generated for each of the 16 readout probes. Each mRNA is expected to have a signal for 4 of the 16 readout probes. FIG. 10H. The images are decoded by determining if a spot is positive (shown as 1) or negative (shown as zero) for a readout probe. Each mRNA has a specific binary readout code.

FIG. 11—Circular map of vector used to insert each guide RNA and generate lentivirus.

FIG. 12—(SEQ ID NO: 1-9) Full sequence map of vector used to insert each guide RNA and generate lentivirus.

FIG. 13—Matched images of transcriptome (left, MERFISH) and Perturbation (right) in a mouse tumor xenograft. The perturbations are shown as one large spot in the nucleus of the cell, the transcriptome as a large number of smaller spots distributed throughout the cell. These example images are from one single Z (out of 7), one single round of images. Scale bars: 10 um.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Key advantages of genetic screens include understanding gene interactions in healthy cells to understand disfunction in disease, stepping away from correlation approaches and establishing causality, and highly multiplexed protocols for the generation of hypotheses. Studying gene networks in space is important because tissues are highly structured, and gene expression is dependent on spatial context, such as cell density and cell neighborhood. Further, gene expression can be paired with multimodal measurements of live cell function.

Embodiments disclosed herein provide methods and kits for performing perturbation screening assays in a plurality of cells that maintain their spatial organization, such that the perturbations are multiplexed and can be both identified in each spatially resolved cell and paired with gene expression for each spatially resolved cell (perturb-FISH). In example embodiments, a set including more than one perturbation (e.g., multiple perturbations) are assayed in a plurality of cells in a single experiment. In example embodiments, the transcriptome identified for the plurality of cells includes more than one gene (e.g., genes are multiplexed). As used herein the term “transcriptome” refers to transcript molecules from a cell or population of cells. In example embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 10%, 25%, 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In example embodiments, the perturbation is encoded for on a perturbation construct integrated into the genome of a cell. The perturbation construct advantageously includes a promoter for expression in live cells to allow perturbation of the cells and includes a promoter to allow in vitro transcription amplification of the perturbation in fixed cells to allow the perturbation sequence to be reliably detected optically in single fixed cells. MERFISH cannot detect single perturbations optically because the perturbation sequence is too short. MERFISH requires multiple probes per mRNA sequence to obtain the necessary signal for optical identification. In example embodiments, the perturbations and gene expression are both optically detected on a plurality of fixed cells. In example embodiments, live perturbed cells are recorded with live cell imaging before determining the perturbation and gene expression (i.e., a spatially resolved movie of the plurality of cells is made). In example embodiments, the method can be performed in combination with any microscopy cell assay.

In example embodiments, the methods and kits allow blocking the expression of one gene per cell and recording the effect of this perturbation on the expression of other genes in many cells using an all-optical approach. This allows access to spatial information, which gives access to cell extrinsic effects, such as the effect of perturbing a gene in a cell on its neighbors, or the effect of cell density on this perturbation. Applicants overcame the obstacle of being able to record both the identity of the perturbation (e.g., guide RNA) in a cell, and the transcriptome using a microscope. Prior to this invention there were no technologies that allowed this. Applicants developed the disclosed chemistries to identify both perturbations and transcriptome optically on the same cells and developed computational tools to decode the images.

In example embodiments, the technology can be applied to any in vitro biological model to screen the effect of blocking a gene on the transcriptome with a cell, and the cells around it. In example embodiments, the technology is a useful tool to study cell-to-cell interactions in vitro, as well as combinations of Perturb-seq like measurements and optical characterizations of cells, such as morphology or temporal signaling. In example embodiments, this requires the ability to grow the cell type(s) of interest on glass and selecting which genes to perturb and measure. In example embodiments, it is necessary to clone guide RNAs into the vector disclosed herein, make lentiviruses, infect the cells of interest, and apply the perturb-FISH protocol to these cells. In example embodiments, perturbation can be performed in vivo and perturbed tissue samples fixed to slides.

Applicants disclose a map of the plasmid vector in which the guide RNAs can be cloned for use in the described technology. The described methods and kits are applicable to both fundamental and industrial research to investigate and define gene networks.

### Methods and Compositions for Performing Multiplexed Optical Spatially Resolved Perturbation Screening

In one aspect, the present invention provides for a method for perturbation screening with spatially resolved readouts comprising: (a) perturbing a plurality of cells by introducing one or more perturbation constructs to the plurality of cells, each perturbation construct encoding for one or more sequence specific perturbations, wherein the one or more sequence specific perturbations comprise a perturbation sequence identifying the perturbation, said sequence operably linked to at least two promoters comprising a Pol III promoter and a phage promoter, and wherein the plurality of cells maintains a spatial localization; (b) fixing the perturbed cells, whereby the spatial localization of the plurality of cells is fixed; (c) contacting the fixed cells with one or more phage polymerases and reagents for in vitro transcription of the perturbation sequences; (d) encoding the plurality of cells by contacting the plurality of cells with: (i) encoding probes specific for a perturbation sequence, each probe comprising a targeting sequence specific for one perturbation sequence and one or more readout sequences specific for each perturbation; and (ii) encoding probes specific for mRNAs expressed in the plurality of cells, each probe comprising a targeting sequence specific for an mRNA sequence and one or more readout sequences specific for each mRNA sequence; (e) contacting the fixed and encoded plurality of cells with fluorescently labeled readout probes specific for a readout sequence and acquiring spatially resolved images for each readout probe using a microscope; and (f) decoding the perturbation and mRNA expression for each cell in the plurality of cells based on the images acquired.

In another aspect, the present invention provides for a kit comprising a library of perturbation constructs, each perturbation construct encoding for one or more sequence specific perturbations, wherein the one or more sequence specific perturbations comprise a perturbation sequence identifying the perturbation, said sequence operably linked to at least two promoters comprising a Pol III promoter and a phage promoter. In certain embodiments, the one or more sequence specific perturbations comprises a CRISPR system. In certain embodiments, the perturbation sequence is a guide sequence. In certain embodiments, each perturbation construct is a viral vector, optionally, a lentiviral vector. In certain embodiments, the kit further comprises encoding probes specific for a perturbation sequence, each probe comprising a targeting sequence specific for one perturbation sequence and one or more readout sequences specific for each perturbation. In certain embodiments, the kit further comprises encoding probes specific for mRNA sequences, each probe comprising a targeting sequence specific for an mRNA sequence and one or more readout sequences specific for each mRNA sequence. In certain embodiments, the perturbation constructs comprise one or more anchor sequences downstream of the perturbation sequence, wherein the one or more anchor sequences are the same for every perturbation construct. In certain embodiments, the kit further comprises acrydite-modified anchor probes comprising a sequence specific to the one or more anchor sequences downstream of the perturbation sequence. In certain embodiments, the kit further comprises acrydite-modified anchor probes comprising a poly(dT) sequence. In certain embodiments, the anchor probes comprise locked nucleic acids (LNAs). In certain embodiments, the kit further comprises fluorescently labeled readout probes specific for a readout sequence on the encoding probes.

### Plurality of Cells

In example embodiments, perturbation constructs are delivered to a plurality of cells to obtain perturbed cells. In example embodiments, any method of introducing DNA constructs to a cell is used (e.g., any method of transfection, transduction). In preferred embodiments, the perturbation construct is delivered using a viral vector (e.g., lentiviral vector). Methods of packaging viral particles is well known in the art.

In example embodiments, a plurality of cells that maintains a spatial localization is perturbed. As used herein “maintains spatial localization” refers to cells that are grown on a solid support or grown in vivo such that the cells maintain their positions relative to all other cells. For example, the plurality of cells can be grown on a slide or cover slip. For example, the cells can be part of a tissue in an organism, a tissue explant, an organoid grown in a cell matrix. The plurality of cells can include any adherent tissue culture cell line or any primary cell line.

In example embodiments, the cells are grown at high density greater than 3,000 cells/cm², 4,000 cells/cm², or 5,000 cells/cm²; or about 10⁷ cells/mL; or about 90-100% confluence. In example embodiments, the cells are grown at low density less than 50 cells/cm², 100 cells/cm², or 200 cells/cm²; or about 10⁵ cells/mL or 10⁴ cells/mL; or about 50% confluence. In example embodiments, the cells are grown at a high density, the high density is greater than 1,000 cells/cm², about 2,000 cells/cm², about 3,000 cells/cm², about 4,000 cells/cm², about 5,000 cells/cm², about 6,000 cells/cm², about 7,000 cells/cells/cm², about 8,000 cells/cells/cm², about 9,000 cells/cm², about 10,000 cells/cm², about 11,000 cells/cells/cm², about 12,000 cells/cm², about 13,000 cells/cm², about 14,000 cells/cm², about 15,000 cells/cells/cm², about 16,000 cells/cm², about 17,000 cells/cm², about 18,000 cells/cm², about 19,000 cells/cells/cm², about 20,000 cells/cm², about 21,000 cells/cm², about 22,000 cells/cm², about 23,000 cells/cells/cm², about 24,000 cells/cm², about 25,000 cells/cm², about 26,000 cells/cm², about 27,000 cells/cells/cm², about 28,000 cells/cm², about 29,000 cells/cm², about 30,000 cells/cm², about 31,000 cells/cells/cm², about 32,000 cells/cm², about 33,000 cells/cm², about 34,000 cells/cm², about 35,000 cells/cells/cm², about 36,000 cells/cm², about 37,000 cells/cm², about 38,000 cells/cm², about 39,000 cells/cm², about 40,000 cells/cm², about 41,000 cells/cm², about 42,000 cells/cm², about 43,000 cells/cm², about 44,000 cells/cm², about 45,000 cells/cm², about 46,000 cells/cm², about 47,000 cells/cm², about 48,000 cells/cm², about 49,000 cells/cm², about 50,000 cells/cm², about 51,000 cells/cm², about 52,000 cells/cm², about 53,000 cells/cm², about 54,000 cells/cm², about 55,000 cells/cm², about 56,000 cells/cm², about 57,000 cells/cm², about 58,000 cells/cm², about 59,000 cells/cm², about 60,000 cells/cm², about 61,000 cells/cm², about 62,000 cells/cm², about 63,000 cells/cm², about 64,000 cells/cm², about 65,000 cells/cm², about 66,000 cells/cm², about 67,000 cells/cm², about 68,000 cells/cm², about 69,000 cells/cm², about 70,000 cells/cm², about 71,000 cells/cm², about 72,000 cells/cm², about 73,000 cells/cm², about 74,000 cells/cm², about 75,000 cells/cm², about 76,000 cells/cm², about 77,000 cells/cm², about 78,000 cells/cm², about 79,000 cells/cm², about 80,000 cells/cm², about 81,000 cells/cm², about 82,000 cells/cm², about 83,000 cells/cm², about 84,000 cells/cm², about 85,000 cells/cm², about 86,000 cells/cm², about 87,000 cells/cm², about 88,000 cells/cm², about 89,000 cells/cm², about 90,000 cells/cm², about 91,000 cells/cm², about 92,000 cells/cm², about 93,000 cells/cm², about 94,000 cells/cells/cm², about 95,000 cells/cm², about 96,000 cells/cm², about 97,000 cells/cm², about 98,000 cells/cm², about 99,000 cells/cm², or about 100,000 cells/cm². In example embodiments, the cells are grown at a high density, the high density is greater than about 10⁶ cells/mL, about 10⁷ cells/mL, or about 10⁸ cells/mL. In example embodiments, the cells are grown at a high density, the high density is 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or greater, or 100% confluence.

In an embodiment, the cells are grown at low density less than 50 cells/cm², 100 cells/cm², or 200 cells/cm²; or about 10⁵ cells/mL or 10⁴ cells/mL; or about 50% confluence. In an embodiment, the cells are grown at low density, the low density less than about 10 cells/cm², about 20 cells/cm², about 30 cells/cm², about 40 cells/cm², about 50 cells/cm², about 60 cells/cm², about 70 cells/cm², about 80 cells/cm², about 90 cells/cm², about 100 cells/cm², about 110 cells/cm², about 120 cells/cm², about 130 cells/cm², about 140 cells/cm², about 150 cells/cm², about 160 cells/cm², about 170 cells/cm², about 180 cells/cm², about 190 cells/cm², about 200 cells/cm², about 210 cells/cm², about 220 cells/cm², about 230 cells/cm², about 240 cells/cm², about 250 cells/cm², about 260 cells/cm², about 270 cells/cm², about 280 cells/cm², about 290 cells/cm², or about 300 cells/cm². In an embodiment, the cells are grown at low density less than about 10⁶ cells/mL, 10⁵ cells/mL, 10⁴ cells/mL, or 10³ cells/mL. In an embodiment, the cells are grown at low density less than about 60%, about 50%, about 40%, about 30%, or about 25%.

In example embodiments, the perturbed cells are fixed. Various fixing methods can be used. In one example embodiment, fixing is accomplished by crosslinking. Non-limiting methods of crosslinking are known in the art. Fixation methods can be divided into two groups: additive and denaturing fixation. Additive fixation solutions (also called cross-linking fixations) contain various aldehydes, including formaldehyde, paraformaldehyde, glutaraldehyde, etc., and can create covalent chemical bonds between proteins. This method can preserve the natural structure of proteins, i.e., secondary and tertiary structures. Another group is the denaturing (or precipitating) fixations. These methods can denature proteins by reducing their solubility and/or disrupting the hydrophobic interactions, and thus modify the tertiary structures of proteins as well as inactivate enzymes. Alcohols, such as methanol and ethanol, are commonly used for denaturing fixation. However, alcohols are seldom solely applied since they can induce serious cell shrinkage. Other denaturing chemicals, like acetone and acetic acid, are usually combined with alcohols to enhance the fixation performance. Common fixation solutions include 2.5% glutaraldehyde, 4-10% formalin (formalin is an alternative name for an aqueous solution of formaldehyde), 4% paraformaldehyde, methanol/acetone (1:1), and ethanol/acetic acid (3:1). Techniques for fixing cells and tissues are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE).

### Perturbations

In example embodiments, a plurality of cells is perturbed with sequence specific perturbations. As used herein “sequence specific” refers to a perturbation that targets a specific nucleotide sequence in a cell (e.g., a DNA or RNA sequence). As used herein the term “perturbation” refers to any alteration of the function of a biological system by external or internal means, such as alterations in gene expression, alterations by environmental stimuli, or alterations by drug treatment. In example embodiments, the perturbation is genetic, chemical, or biological. In example embodiments, perturbations are identified by a barcode that can be detected by sequential rounds of single-molecule FISH (smFISH). In the most commonly used smFISH technique, detection of an individual RNA molecule requires multiple short DNA probes (often ~48 20-mer probes) that are complementary to the target RNA and are conjugated to the same fluorescent dye. Binding of a single fluorescent probe results in weak signal, but the signal from the ensemble of all the probes is robust. This feature greatly improves the signal-to-noise ratio because even though a single probe can exhibit off-target binding, such signal is expected to be very weak compared to that of the target RNA molecule (Chen J, McSwiggen D, Ünal E. Single Molecule Fluorescence In Situ Hybridization (smFISH) Analysis in Budding Yeast Vegetative Growth and Meiosis. J Vis Exp. 2018 May 25; (135):57774).

In example embodiments, the perturbation is a genetic perturbation (e.g., CRISPR, INDELs, substitutions, CRISPRa (CRISPR activation), CRISPRi (CRISPR interference), RNAi (RNA interference), and base editor mediated mutagenesis). As used herein a genetic perturbation refers to a perturbation that perturbs a nucleic acid, such as a genome sequence (e.g., a target gene or regulatory element) or RNA sequence (e.g., a transcript sequence). As used herein a chemical perturbation refers to a perturbation such as a small molecule, compound, or drug (e.g., a chemotherapy). As used herein a biological perturbation refers to a perturbation such as a biologic drug (e.g., antibody or peptide). In example embodiments, the perturbations can be identified by a barcode sequence or barcode sequences. In example embodiments, the one or more perturbations target specific genes of interest (e.g., kinases, GPCRs, pathways specific genes). In example embodiments, the one or more perturbations are genome-wide perturbations.

### Vectors

In example embodiments, the sequence specific perturbation is encoded for by a vector. In example embodiments, the vector encoding for one or more sequence specific perturbations comprises a perturbation sequence identifying the perturbation and preferably encoding the perturbation. In example embodiments, the perturbation sequence is operably linked to at least two promoters comprising a Pol III promoter and a phage promoter (or any promoter capable of use in in vitro transcription). In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome (e.g., lentivirus). Moreover, certain vectors are capable of directing the expression of genes to which they are operatively linked (i.e., operably linked to a regulatory element). Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids. The term “regulatory element” is intended to include promoters, enhancers, internal ribosomal entry sites (IRES), and other expression control elements (e.g., transcription termination signals, such as polyadenylation signals and poly-U sequences). In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the 3-actin promoter, the phosphoglycerol kinase (PGK) promoter, and the EF1α promoter. In example embodiments, a phage promoter is included in the perturbation construct to allow for in vitro transcription of the perturbation sequence in fixed cells, preferably, the phage promoter includes T3, T7 or SP6 promotor sequences or derivatives thereof. As used herein an in vitro transcribed RNA is an RNA molecule that has been synthesized from a template DNA, commonly a linearized and purified plasmid template DNA, a PCR product, or an oligonucleotide, but also includes fixed genomic DNA in a fixed cell. RNA synthesis occurs in a live cell free (“in vitro”) assay catalyzed by DNA dependent RNA polymerases. Particular examples of DNA dependent RNA polymerases are the T7, T3, and SP6 RNA polymerases. In preferred embodiments, the perturbation construct includes a pol III promoter to express the perturbation sequence in live cells and a phage promoter for in vitro transcription in fixed cells. Examples of a U6T7 promoter applicable to the present invention have been described (see, e.g., Romanienko P J, Giacalone J, Ingenito J, et al. A Vector with a Single Promoter for In Vitro Transcription and Mammalian Cell Expression of CRISPR gRNAs. PLoS One. 2016; 11(2):e0148362).

Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

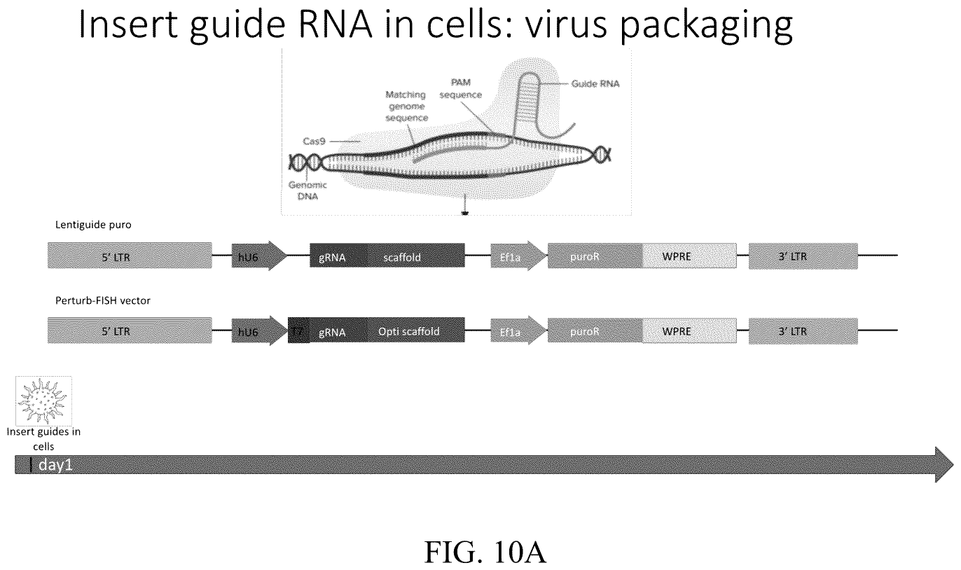

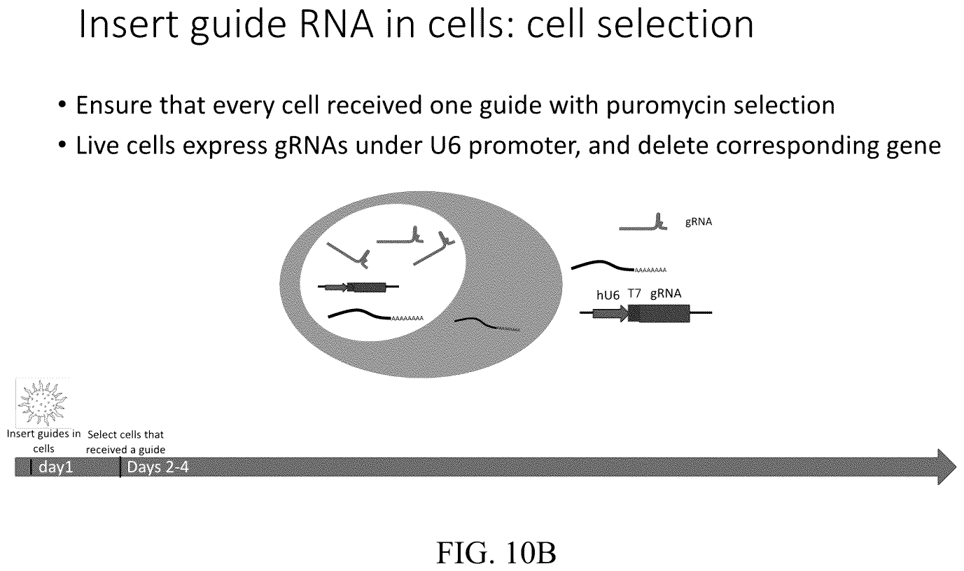

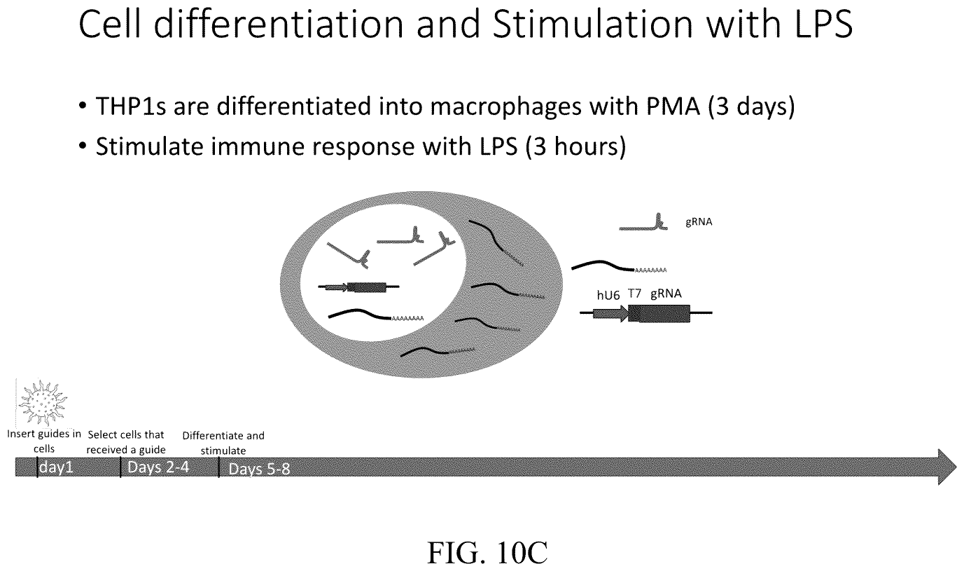

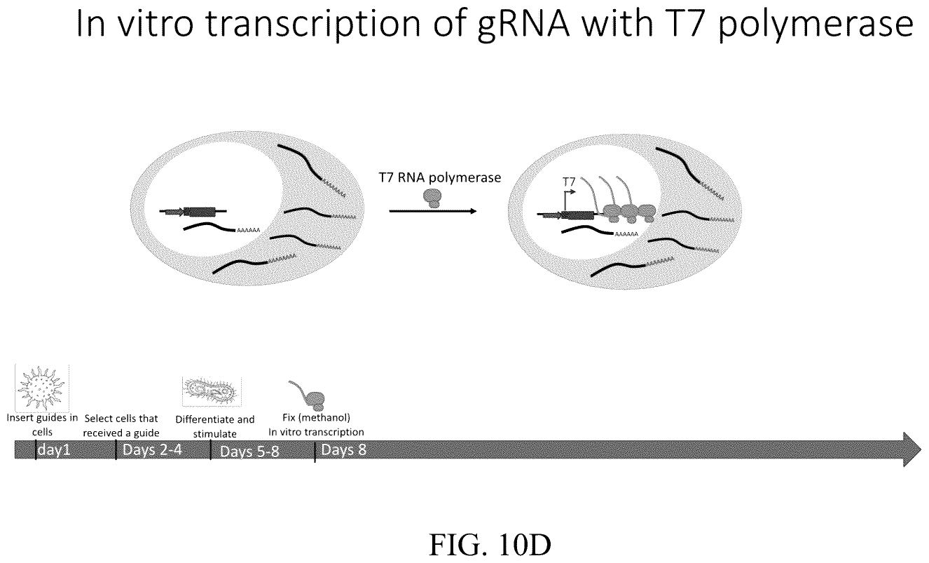

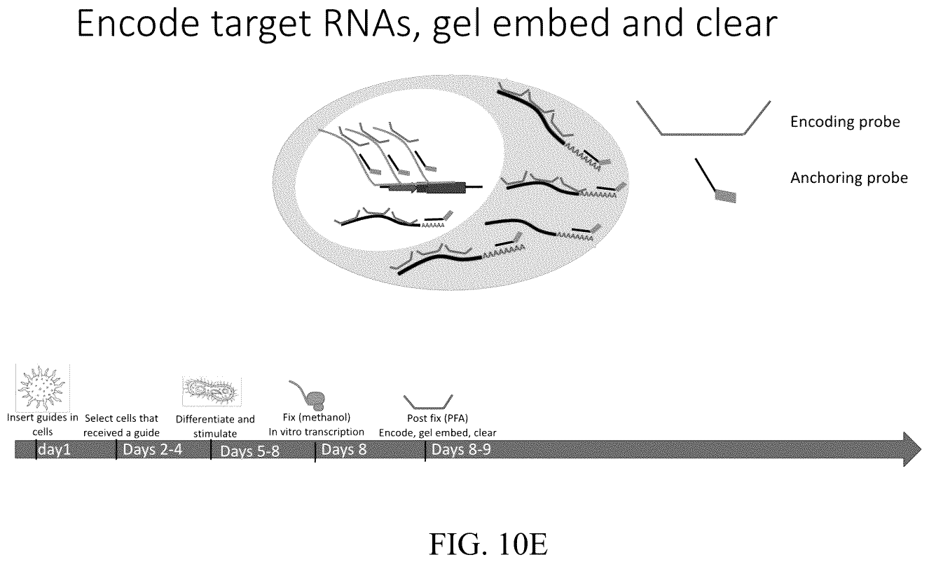

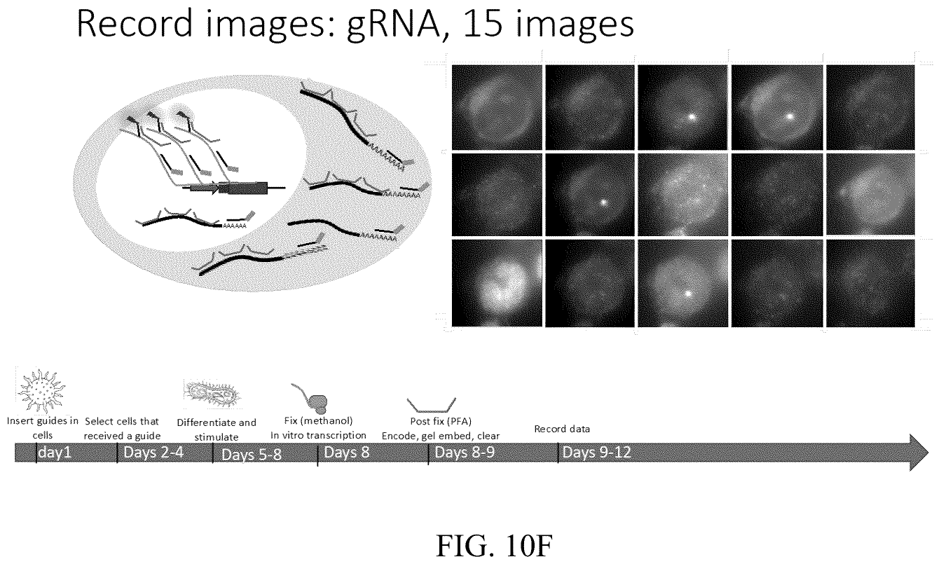

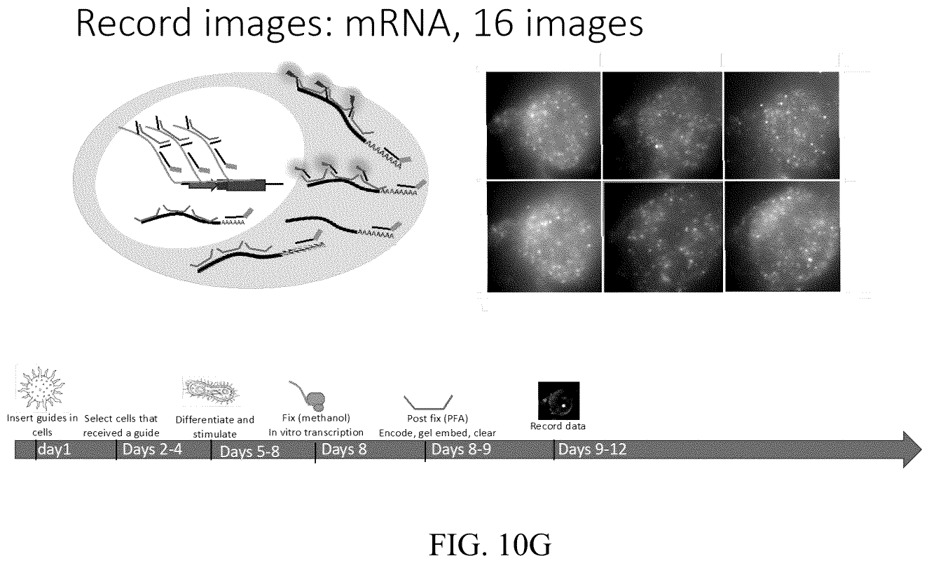

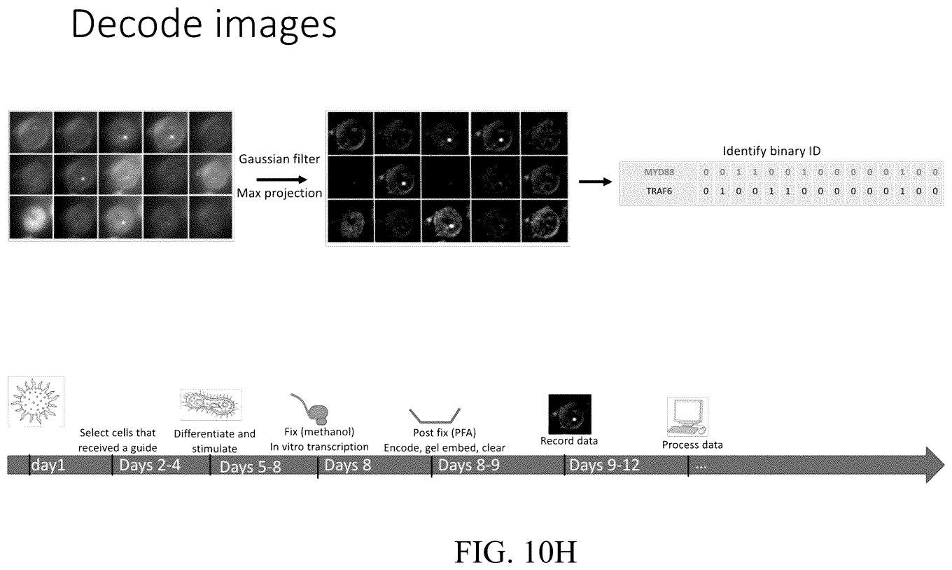

Advantageous vectors include lentiviruses and adeno-associated viruses, and types of such vectors can also be selected for targeting particular types of cells. An exemplary vector is provided for in FIGS. 10, 11, and 12.

In an example embodiment, the one or more perturbation constructs are encoded into for by a viral vector and the one or more perturbation constructs are introduced to a plurality of cells by the viral vector. In an example embodiment, the viral vector introduces the one or more perturbation constructs to a plurality of cells in vivo. In an embodiment, the viral vector introduces the one or more perturbation constructs to a plurality of cells ex vivo and the externally transfected cells are then delivered two a second plurality of cells in vivo.

### Xenografts and Transplantation

Xenografts are a material that include cells, tissues, and/or organs of one species, which can be used as transplant material for another species. This technique may also be referred to as xenotransplantation. In an embodiment, methods described herein further include xenotransplantation. In an embodiment, one or more perturbation constructs is introduced to one or more cell, tissue, and/or organ of one species using any of the methods described herein. The transfected one or more cell, tissue, and/or organ of one species can be xenografted to another species. See e.g., A. N. Carrier, et al., Xenotransplantation: A New Era. Frontiers in Immunology, 2022, 13.

In an embodiment, one or more transplantation methods are used. For example, allotransplantation, syngeneic transplantation, isotransplantation, and autotransplantation.

### Barcodes and Detection of Barcodes

In example embodiments, perturbations and mRNAs are identified in single fixed cells optically using perturbation specific binary barcodes. The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin, sample of origin, or individual transcript. In situ imaging-based approaches to single-cell transcriptomics allow not only the expression profile of individual cells to be determined, but also the spatial positions of individual RNA molecules to be localized. These approaches provide powerful means to map the spatial organizations of RNAs inside cells and the transcriptionally distinct cells in tissues. Currently available image-based single-cell RNA profiling methods rely on either multiplexed fluorescence in situ hybridization (FISH) or in situ sequencing. In particular, multiplexed error-robust FISH (MERFISH), a massively multiplexed form of single-molecule FISH (smFISH), allows RNA imaging at the transcriptomic scale. As a powerful method that images individual RNA molecules inside cells, smFISH provides the precise copy number and spatial organization of RNAs in single cells. MERFISH multiplexes smFISH measurements by labeling RNAs combinatorically with oligonucleotide probes (referred to as encoding probes herein) which contain error-robust barcodes and measuring these barcodes through sequential rounds of smFISH imaging (i.e., using readout probes). In example embodiments, the encoding probes may include smFISH or MERFISH probes, such as those discussed in Int. Pat. Apl. Pub. Nos. WO 2016/018960 or WO 2016/018963, each incorporated herein by reference in its entirety. Using this approach, simultaneous imaging of hundreds to thousands of RNA species in individual cells using error detection/correction barcoding schemes have been demonstrated. See, e.g., U.S. Pat. Apl. Pub. No. US20170220733, entitled “Systems and Methods for Determining Nucleic Acids,” and U.S. Pat. Apl. Pub. No. US20170212986, entitled “Probe Library Construction,” each incorporated herein by reference in its entirety.

Oligonucleotide probes which contain error-robust barcodes are referred to herein as encoding probes. In example embodiments, the encoding probes comprises a targeting sequence that hybridizes to a target RNA and comprises readout sequences, which act as a barcode sequence because each encoding probe targeting different RNA sequences includes different combinations of readout sequences. In example embodiments, the encoding probes include, 2, 3, 4, 5, 6, 7, or up to 8 readout sequences. In example embodiments, the sequence targeted by the encoding probe is also referred to as the barcode sequence (i.e., the perturbation sequence) because this sequence is targeted by the encoding probe and identifies the perturbation. In example embodiments, the DNA sequence encoding the guide sequence is the perturbation sequence (i.e., barcode). In example embodiments, the combination of readout sequences is specific to a guide RNA or an mRNA and is also referred to as a barcode. In example embodiments, sets of encoding probes include encoding probes specific to perturbations and encoding probes specific to mRNAs. In example embodiments, the encoding probes for mRNA include different readout sequences than encoding probes for perturbations. In this way, the perturbations can be imaged separately from mRNA sequences. In example embodiments, the number of different readout sequences depends on the number of sequences to be identified. For example, the perturbations may only require 5, 10, 15, or 20 readout sequences to be able to identify the perturbations and 5, 10, 15, or 20 images would be required to identify all of the perturbations. For example, Applicants detect 35 perturbations using combinations of four readout sequences for each encoding probe selected from 15 separate readout sequences. Applicants detect 15 separate images, one for each readout sequence. In the present invention, a single encoding probe is used to detect each perturbation identity, in contrast to mRNA that is detected by many encoding probes. Applicants discovered that amplification of the perturbation sequence allows for the single encoding probe to detect a guide sequence without background noise from off-target binding. In example embodiments, detection of the perturbation is imaged separately from mRNA imaging due to the difference in how each is detected.

A variety of techniques may be used to determine binding, including optical techniques such as fluorescence microscopy. In some cases, spatial positions may be determined at super resolutions, or at resolutions better than the wavelength of light or the diffraction limit (although in other embodiments, super resolutions are not required). For example, techniques such as STORM (stochastic optical reconstruction microscopy) may be used. See, for example, U.S. Pat. No. 7,838,302, issued Nov. 23, 2010, entitled “Sub-Diffraction Limit Image Resolution and Other Imaging Techniques,” by Zhuang, et al., incorporated herein by reference in its entirety.

In example embodiments, detection of perturbation sequences and mRNA can be improved by embedding the fixed cells in a polymerized hydrogel and anchoring the target RNA sequences to the polymerized hydrogel. See, e.g., U.S. Pat. Apl. Pub. No. US20190276881A1. In example embodiments, the fixed and embedded cells are cleared to reduce background. In example embodiments, the polymerized hydrogel can be swelled or expanded to improve detection of barcodes. In example embodiments, the measurement throughput of MERFISH to tens of thousands of cells per single-day-long measurement may be increased. In example embodiments, sample clearing approaches are used that increase the signal-to-background ratio by anchoring cellular RNAs to a polymer matrix and removing other cellular components that give rise to fluorescence background have been developed, and these clearing approaches allows high-quality MERFISH measurement of tissue sections. See, e.g., U.S. Pat. Apl. Pub. No. US20190264270, entitled “Matrix Imprinting and Clearing”, incorporated herein by reference in its entirety.

In example embodiments, anchor probes are contacted to the fixed cells in combination with encoding probes. As used herein anchor probes are sequences that hybridize to sequences present on perturbation constructs (anchor sequences) or mRNA sequences and include a moiety that is capable of being linked to the hydrogel during polymerization. In example embodiments, the anchor probes are specific to anchor sequences present on the perturbation constructs. The anchor sequences are the same across all perturbation constructs. In example embodiments, anchor probes are specific for hybridizing to poly-A tailed mRNA and comprise a poly-dT sequence.

In example embodiments, anchor probes may be used during the polymerization process. The anchor probes may include an anchor portion that is able to polymerize with the expandable material, e.g., during and/or after the polymerization process, and a targeting portion that is able to immobilize a target, e.g., chemically and/or physically. For example, in the case of polyacrylamide, the anchor probe may include an acrydite portion that can polymerize and become incorporated into the polymer. As another example, an anchor probe may contain, as a targeting portion, a sequence of nucleic acids that is complementary to a target that is a nucleic acid, such as RNA (e.g., mRNA) or DNA. The targeting portion may be specific to a target, and/or may randomly associate with different targets within a sample (for example, due to non-specific binding). Other portions may be present within the anchor probes as well.

For example, to associate with a target nucleic acid, the anchor probe may comprise a nucleic acid sequence substantially complementary to at least a portion of the target nucleic acid. For instance, the nucleic acid may be complementary to at least 5, 6, 7, 8, 9, 10, 11, 12, 13, 14, 15, 16, 17, 18, 19, or 20 or more nucleotides of the nucleic acid. In some cases, the complementarity may be exact (Watson-Crick complementarity), or there may be 1, 2, or more mismatches.

Thus, the anchor probe may contain a portion that can interact with and bind to nucleic acid molecules in some embodiments, and/or other molecules in which immobilization is desired, e.g., proteins or lipids, other desired targets, etc. The immobilization may be covalent or non-covalent. For example, to immobilize a target nucleic acid, the anchor probe may comprise a nucleic acid comprising an acrydite portion (e.g., at the 5′ end, the 3′ end, an internal base, etc.), and a portion able to recognize the target nucleic acid.

In some cases, the anchor probe can be configured to immobilize mRNA, e.g., in the case of transcriptome analysis. For instance, in one set of embodiments, the anchor probe may contain a plurality of thymine nucleotides, e.g., sequentially, for binding to the poly-A tail of an mRNA. Thus, for example, the anchor probe can have at least 5, 6, 7, 8, 9, 10, 11, 12, 13, 14, 15, 16, 17, 18, 19, or 20 or more consecutive thymine nucleotides (e.g., a poly-dT portion) within the anchor probe. In some cases, at least some of the thymine nucleotides may be “locked” thymine nucleotides. These may comprise at least 20%, at least 30%, at least 40%, at least 50%, at least 60%, at least 70%, or at least 80% of these thymine nucleotides. In certain embodiments, the locked and non-locked nucleotides may alternate. Such locked thymine nucleotides may be useful, for example, to stabilize the hybridization of the poly-A tails of the mRNA with the anchor probe.

In another set of embodiments, the anchor probe may comprise a sequence substantially complementary to mRNA (or another target nucleic acid), as noted above. The sequence may be substantially complementary to all, or only a portion, of the target nucleic acid, for example, an end portion (e.g., towards a 5′ end or a 3′ end), or a middle portion between the end portions. For example, a nucleic acid may be immobilized using anchor probes having substantially complementary portions to the DNA or RNA target. There may be, e.g., 5 or more, 6 or more, 7 or more, 8 or more, 9 or more, 10 or more, 12 or more, 13 or more, 14 or more, 15 or more, 20 or more, 25 or more, 30 or more, 35 or more, 40 or more, 45 or more, or 50 or more complementary nucleotides between the anchor probe and the nucleic acid.

Other methods may be used to anchor nucleic acids, or other molecules in which immobilization is desired. In one set of embodiments, nucleic acids such as DNA or RNA may be immobilized by covalent bonding. For example, in one set of embodiments, an alkylating agent may be used that covalently binds to RNA or DNA and contains a second chemical moiety that can be incorporated into the polyelectrolytes as it is polymerized. In yet another set of embodiments, the terminal ribose in an RNA molecule may be oxidized using sodium periodate (or another oxidizing agent) to produce an aldehyde, which may be cross-linked to acrylamide, or other polymer or gel. In other embodiments, chemical agents that are able to modify bases may be used, such as aldehydes, e.g. paraformaldehyde or gluteraldehyde, alkylating agents, or succinimidyl-containing groups; chemical agents that modify the terminal phosphate, such as carbodiimides, e.g., EDC (1-ethyl-3-(3-dimethylaminopropyl)carbodiimide); chemical agents that modify internal sugars, such as p-maleimido-phenyl isocyanate; or chemical agents that modify terminal sugars, such as sodium periodate. In some cases, these chemical agents can carry a second chemical moiety that can then be directly cross-linked to the gel or polymer, and/or which can be further modified with a compound that can be directly cross linked to the gel or polymer.

In example embodiments, any nucleic acid probe described herein may comprise nucleic acids (or entities that can hybridize to a nucleic acid, e.g., specifically) such as DNA, RNA, LNA (locked nucleic acids), PNA (peptide nucleic acids), or combinations thereof. In some cases, additional components may also be present within the nucleic acid probes. In example embodiments, the cell or other sample is fixed prior to introducing the nucleic acid probes, e.g., to preserve the positions of the nucleic acids within the sample.

In example embodiments, the fixed cells are embedded in a polymerized gel that is a non-swellable hydrogel. In example embodiments, the fixed cells are embedded in a polymerized gel that is a swellable hydrogel. As used herein, the term “swellable material” generally refers to a material that expands when contacted with a liquid, such as water or other solvent. Preferably, the swellable material uniformly expands in three dimensions. Additionally or alternatively, the material is transparent such that, upon expansion, light can pass through the sample. Preferably, the swellable material is a swellable polymer or hydrogel. In one embodiment, the swellable material is formed in situ from precursors thereof. For example, one or more polymerizable materials, monomers or oligomers can be used, such as monomers selected from the group consisting of water-soluble groups containing a polymerizable ethylenically unsaturated group. Monomers or oligomers can comprise one or more substituted or unsubstituted methacrylates, acrylates, acrylamides, methacrylamides, vinylalcohols, vinylamines, allylamines, allylalcohols, including divinylic crosslinkers thereof (e.g., N, N-alkylene bisacrylamides). Precursors can also comprise polymerization initiators and crosslinkers.

In a preferred embodiment, the swellable polymer is polyacrylate and copolymers or crosslinked copolymers thereof. Alternatively or additionally, the swellable material can be formed in situ by chemically crosslinking water-soluble oligomers or polymers. Thus, the invention envisions adding precursors (such as water-soluble precursors) of the swellable material to the sample and rendering the precursors swellable in situ.

Preferably, “embedding” the sample in a swellable material comprises permeating (such as, perfusing, infusing, soaking, adding or other intermixing) the sample with the swellable material, preferably by adding precursors thereof. Alternatively or additionally, embedding the sample in a swellable material comprises permeating one or more monomers or other precursors throughout the sample and polymerizing and/or crosslinking the monomers or precursors to form the swellable material or polymer. In this manner the sample of interest is embedded in the swellable material. Preferably a sample of interest, or a labeled sample, is permeated with a composition comprising water soluble precursors of a water swellable material and reacting the precursors to form the water swellable material in situ.

In example embodiments, the fixed cells are embedded in a non-swellable material. In example embodiments, embedding the sample in a non-swellable material comprises permeating one or more monomers or other precursors throughout the sample and polymerizing and/or crosslinking the monomers or precursors to form the non-swellable material or polymer. In example embodiments, “re-embedding” the expanded sample comprises permeating (such as, perfusing, infusing, soaking, adding or other intermixing) the sample with the non-swellable material, preferably by adding precursors thereof. In this manner the first enlarged sample, for example, is embedded in the non-swellable material. The non-swellable material can be charge-neutral hydrogels. For example, it can be polyacrylamide hydrogel, composed of acrylamide monomers, bisacrylamide crosslinker, ammonium persulfate (APS) initiator and tetramethylethylenediamine (TEMED) accelerator.

As mentioned, in one aspect, a sample is embedded or contained within an expandable material. The sample may be any suitable sample and may be biological in some embodiments. In some cases, the sample contains DNA and/or RNA, e.g., that may be determined within the sample. (In other embodiments, other targets within the sample may be determined.) In some cases, the sample may include cells, such as mammalian cells (including human cells), or other types of cells. The sample may contain viruses in some cases. In addition, in some cases, the sample may be a tissue sample, e.g., from a biopsy, artificially grown or cultured, etc.

In example embodiments, the expandable material is one that can be expanded, for example, when exposed to water or another suitable liquid. For example, the material may exhibit a relative change in size of at least 1.1, at least 1.2 at least 1.3, at least 1.5, at least 2, at least 3, at least 4, at least 5, at least 7, at least 10, or at least 15, etc., and/or a relative change in size that is less than 15, less than 10, less than 7, less than 5, less than 4, less than 3, less than 2, less than 1.5, less than 1.3, or less than 1.2 (i.e., a change in size of 2 means that a sample doubles in linear dimension), or inverses of these (i.e., an inverse change in size of 2 means that a sample halves in linear dimensions).

In some embodiments, the expandable material may be one that does not significantly distort during the expansion process (e.g., the expandable material may expand substantially uniformly or isotropically in all 3 dimensions), although in some cases, the expandable material may exhibit some distortion or non-isotropic expansion. For example, the expandable material may expand in one dimension, relative to an orthogonal dimension, by less than 150%, less than 130%, less than 125%, less than 120%, less than 115%, less than 110%, or less than 105% by linear dimension relative to the shorter linear expansion.

In some cases, the expandable material is a polymer. Non-limiting examples of suitable polymers include polyelectrolytes and agarose. In some cases, the polymer is a gel or a hydrogel. A variety of polymers can be used in various embodiments including but not limited to acrylic acid, acrylamide, ethylene glycol diacrylate, ethylene glycol dimetharcrylate, poly(ethylene glycol dimethacrylate), poly(N-isopropyl acrylamide), methyl cellulose, (ethylene oxide)-(propylene oxide)-(ethylene oxide) terpolymers, sodium alginate, poly(vinyl alcohol), alginate, chitosan, gum Arabic, gelatin, agarose, or the like. In some cases, the polymer may be selected to be relatively optically transparent. In some cases, the expandable material may be formed from monomers or oligomers, for example, comprising one or more substituted or unsubstituted methacrylates, acrylates, acrylamides, methacrylamides, vinylalcohols, vinylamines, allylamines, allylalcohols, including divinylic crosslinkers thereof (e.g., N,N-alkylene bisacrylamides such as N,N-methylenebisacrylamide), or the like. In some cases, polymerization initiators and/or crosslinkers may be present. For example, a precursor may include one or more cross-linking agents, which may be used to cross-link a polymeric expandable material as it forms, e.g., during the polymerization process.

After immobilization of nucleic acids, or other suitable molecules, to the polymer or gel, other components within the sample may be “cleared.” Such clearance may include removal of the components, and/or degradation of the components (e.g., to smaller components, components that are not fluorescent, etc.) that are not the desired target. In some cases, at least 50%, at least 60%, at least 70%, at least 80%, or at least 90% of the undesired components within the sample may be cleared. Multiple clearance steps can also be performed in certain embodiments, e.g., to remove various undesired components. As discussed, it is believed that the removal of such components may decrease background during analysis (for example, by decreasing background and/or off-target binding), while desired components (such as nucleic acids) can be immobilized and thus not cleared.

For example, proteins may be cleared from the sample using enzymes, denaturants, chelating agents, chemical agents, and the like, which may break down the proteins into smaller components and/or amino acids. These smaller components may be easier to remove physically, and/or may be sufficiently small or inert such that they do not significantly affect the background. Similarly, lipids may be cleared from the sample using surfactants or the like. In some cases, one or more of these are used, e.g., simultaneously or sequentially. Non-limiting examples of suitable enzymes include proteinases such as proteinase K, proteases or peptidases, or digestive enzymes such as trypsin, pepsin, or chymotrypsin. Non-limiting examples of suitable denaturants include guanidine HCl, acetone, acetic acid, urea, or lithium perchlorate. Non-limiting examples of chemical agents able to denature proteins include solvents such as phenol, chloroform, guanidinium isocyananate, urea, formamide, etc. Non-limiting examples of surfactants include Triton X-100 (polyethylene glycol p-(1,1,3,3-tetramethylbutyl)-phenyl ether), SDS (sodium dodecyl sulfate), Igepal CA-630, or poloxamers. Non-limiting examples of chelating agents include ethylenediaminetetraacetic acid (EDTA), citrate, or polyaspartic acid. In some embodiments, compounds such as these may be applied to the sample to clear proteins, lipids, and/or other components. For instance, a buffer solution (e.g., containing Tris or tris(hydroxymethyl)aminomethane) may be applied to the sample, then removed.

Non-limiting examples of DNA enzymes that may be used to remove DNA include DNase I, dsDNase, a variety of restriction enzymes, etc. Non-limiting examples of techniques to clear RNA include RNA enzymes such as RNase A, RNase T, or RNase H, or chemical agents, e.g., via alkaline hydrolysis (for example, by increasing the pH to greater than 10). Non-limiting examples of systems to remove sugars or extracellular matrix include enzymes such as chitinase, heparinases, or other glycosylases. Non-limiting examples of systems to remove lipids include enzymes such as lipidases, chemical agents such as alcohols (e.g., methanol or ethanol), or detergents such as Triton X-100 or sodium dodecyl sulfate. Many of these are readily available commercially. In this way, the background of the sample may be removed, which may facilitate analysis of the nucleic acid probes or other desired targets, e.g., using fluorescence microscopy, or other techniques as discussed herein. As mentioned, in various embodiments, various targets (e.g., nucleic acids, certain proteins, lipids, viruses, or the like) may be immobilized, while other non-targets may be cleared using suitable agents or enzymes. As a non-limiting example, if a protein (such as an antibody) is immobilized, then RNA enzymes, DNA enzymes, systems to remove lipids, sugars, etc. may be used.

### Genetic Modification Systems

In one example embodiment, the perturbation comprises a genetic modification system to either decrease or increase expression of one or more genes in the plurality of cells. The genetic modifying agent may comprise a programmable nuclease, such as, a CRISPR system, a zinc finger nuclease system, a TALEN, or a meganuclease, or an OMEGA system. In addition, a number of alternate gene modification systems have been developed by modifying Cas nuclease so that they are catalytically inactive (“dead Cas” or “dCas”) or cut only a single strand of DNA (“nickase”) and then coupling these modified Cas nucleases with a further functional domain such as base editors, reverse transcriptases, recombinases, transposases and retrotransposases. For sake of convenience these alternative systems (e.g., Base Editors, Prime Editors, CAST, Non-LTR Retrotransposon Systems, Epigenetic Editors) are described further below in the context of use with a modified Cas. However, it is further contemplated that the modified Cas could be substituted with another similarly modified programmable nuclease like Zinc Finger nucleases, TALENs, Omega nucleases (e.g., Iscb, Isrb, TnpB, Fanzor), or a meganuclease. In example embodiments, the genetic modifying agent is administered using a vector, such as a viral vector or liposome. Programmable nucleases may use two different cell repair pathways to effectuate edits to one or more target sequences, non-homologous end joining (NHEJ) or homology-directed repair (HDR).

### Example NHEJ-Mediated Modifications

Programmable nuclease may be used to introduce insertions and deletions via NHEJ-mediated cell repair that control expression of one or more genes. The modifications may be made in a non-coding region that controls expression of the one or more target genes, in a coding region encoding a gene expression product (e.g., a polypeptide) of the one or more target genes, or a combination thereof. More than one programmable nuclease type may be used, for example and in the case of CRISPR-Cas, to maximize targets sites adjacent to different PAMs.

NHEJ-Mediated Modifications that Decrease Expression by Targeting a Non-Coding Region

In one embodiment, the one or more programmable nucleases may be configured to introduce one or more insertions or deletion in a non-coding region controlling expression of one or more genes such that expression of the one or more genes is reduced. In one embodiment, the insertions or deletions may disrupt the binding site in an enhancer of one or more proteins, such as a transcription factor or other regulatory proteins, needed to initiation transcription of one or more genes. In one embodiment, the one or more insertions or deletions may disrupt one or more promoters controlling expression of one or more genes such that binding of transcription factors and/or RNA polymerase binding is blocked or reduced. In one embodiment, the one or more insertions or deletions may disrupt one or more insulator regions such that silencer regions or repressive chromatin structures controlling expression of the one or more genes are no longer muted or blocked by the insulator region and can decrease gene expression.

NHEJ-Mediated Modification that Increase Expression by Targeting a Non-Coding Region

In one embodiment, the one or more programmable nucleases may be configured to introduce one or more insertions or deletions in a non-coding region controlling expression of one or more genes such that expression of the one or more target genes is increased. In one embodiment, the one or more insertions or deletions modify one or more enhancer regions controlling expression of one or more genes such that binding of transcription factors or other regulatory proteins is increased or strengthened and gene expression is increased. In one embodiment, the one or more insertions or deletions modify one or more promoter regions controlling expression of one or more genes such that binding of transcription factors and/or RNA polymerase is increased or strengthened and gene expression is increased. In one embodiment, the one or more insertions or deletions disrupt one or more silencer regions controlling expression of one or more genes, such that binding of transcriptional repressor is blocked or reduced and gene expression is increased.

NHEJ-Mediated Modification that Decrease Expression by Targeting a Coding Region

In one embodiment, the programmable nuclease is used to introduce one or more insertions or deletions to coding sequence of one or more genes, such that one or more indels or insertions reduce expression or activity of one or more genes. For example, the insertion or deletion may cause a frame shift in the coding sequence such that expression is reduced or such that the resulting gene product is non-functional or exhibits reduced activity relative to an unmodified gene. In one embodiment, the insertion(s) or deletion(s) may alter a splice site such that transcription or translation is reduced or such that a resulting gene product is non-functional or exhibits reduced activity relative to an unmodified gene. The insertion or deletion may introduce a premature stop codon such that expression is reduced. The insertion or deletion may alter a post-translational modification site such that the activity of the resulting gene product is reduced.

NHEJ-Mediated Modification that Increase Expression by Targeting a Coding Region

In one embodiment, the programmable nuclease is used to introduce one or more deletions or insertions in the coding sequence of one or more genes such that expression of the one or more genes is increased. For example, the insertion or deletion may cause a frame shift in the coding sequence such that expression is increased or such that the resulting gene product is exhibits increased activity relative to an unmodified gene. The insertion or deletion may alter a splice site such that transcription or translation is increased or such a that resulting gene product exhibits increased activity relative to an unmodified gene. The insertion or deletion may introduce a premature stop codon such that expression is increased. The insertion or deletion may alter a post-translational modification site such that the activity of the resulting gene product is increased.

### Example HDR-Mediated Modifications

In one example embodiment, a donor template is provided along with a programmable nuclease to facilitate homology direct repair (HDR) which results insertion of a donor sequence comprising one or more insertions, deletions, or substitutions relative to the target sequence it replaces. A donor template may comprise an insertion sequence flanked by two homology regions. The insertion sequence comprises an edited sequence to be inserted in place of the target sequence (e.g., a portion of genomic DNA to be edited). The homology regions comprise sequences that are homologous to the genomic DNA strands at the site of the CRISPR-Cas induced double-strand break. Cellular HDR mechanisms then facilitate insertion of the insertion sequence at the site of the DSB.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

The use of donor templates may be used to introduce insertions, deletions, or substitutions (modifications) that control expression of the one or more genes. The modifications may be made in a non-coding region that controls expression of the one or more target genes, in a coding region encoding a gene expression product (e.g., a polypeptide), or both. Example modifications are described in further detail below.

HDR-Based Modifications that Decrease Expression by Targeting Non-Coding Regions

In one example embodiment, the donor template is configured to introduce a deletion, insertion, or mutation in one or more enhancer regions such that binding of transcription factors or other regulatory proteins controlling expression of the one or more genes is disrupted thereby reducing transcription initiation and gene expression. In one example embodiment, the donor template is configured to introduce a deletion, insertion, or mutation in one or more promoters controlling expression of one or more genes to prevent or disrupt the binding of transcription factors and RNA polymerase such that transcription initiation and gene expression are blocked or reduced. In one example embodiment, the donor template is configured to introduce a silencer element into a non-coding region controlling expression of one or more genes leading to the recruitment of transcriptional repressors that block or decrease gene expression. In one embodiment, the donor template is configured to modify or replace an existing silencer element controlling expression of one or more genes such that the silencing function of the silencer element is increased relative to an unmodified silencer sequence. In another embodiment, the donor template is configured to disrupt or replace one or more insulator sequences controlling expression of one or more genes such that nearby silencer element or repressive chromatin structures decrease gene expression.

HDR-Based Modifications that Increase Expression by Targeting Non-Coding Regions

In one embodiment, the programable nuclease and donor template may be configured to make one or more modifications (insertions, substitutions, deletions) in a non-coding region of one or more genes that result in increased expression of the one or more genes. In one embodiment, the one or more modifications modify one or more enhancer regions controlling expression of one or more genes such that binding of transcription factors or other regulatory proteins is increased or strengthened and gene expression increased. In another embodiment, the one or more modifications modify one or more promoters controlling expression of one or more genes such that binding of transcription factors and/or RNA polymerase is increased or strengthened and gene expression is increased. In another embodiment, the one or modifications disrupt or remove one or more silencer elements that control expression of one or more genes, such that binding of transcriptional repressors is prevented or weakened and gene expression is increased. In another embodiment, the one or more modifications introduce or strengthen insulator sequences controlling expression of the one or more genes thereby reducing the influence of nearby silencer elements or repressive chromatin structures such that gene expression is increased.

HDR-Based Modifications that Decrease Expression by Targeting Coding Regions

In one embodiment, the programmable nuclease and donor template are configured such that one or more modifications (e.g. insertions, deletions, substitutions) are made in a coding region of the one or more genes such that expression of the one or more genes is reduced. In one embodiment, the one or more modifications result in a frame-shift mutation leading to introduction of a premature stop codon and the production of non-functional, truncated proteins or the triggering of nonsense-mediated mRNA decay (NMD) thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to active a proteins function. Accordingly, modifications that introduce inhibitory PTMs or remove activating PTMs may be made to decrease protein function, stability, and/or degradation.

HDR-Based Modifications that Increase Expression by Targeting Coding Regions

In one embodiment, the programmable nuclease and donor template are configured such that one or more modifications (e.g., insertions, deletions, substitutions) are mode in a coding region of the one or more genes such that expression of the one or more genes is increased. In one embodiment, the one or more modifications comprise removing inhibitors sequences, such as IRESs or upstream open reading frames (uORFs), which can negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modification may comprise mutations or modifications within the coding region that improve catalytic activity, binding affinity, or other functional properties of the protein. This approach does not directly increase gene expression but can result in an overall increase in the functional output of the gene product.

### Example Programmable Nucleases

The following provides further details and nuclease specific considerations for example programmable nucleases that may be used to make the NHEJ-mediated and HDR-mediated modifications described above.

### CRISPR-Cas

In one example embodiment, the genetic modifying agent is a CRISPR-Cas system. CRISPR-Cas systems comprise a Cas polypeptide and a guide sequence, wherein the guide sequence is capable of forming a CRISPR-Cas complex with the Cas polypeptide and directing site-specific binding of the CRISPR-Cas sequence to a target sequence in one or more of the target genes. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to a desired target site at or near the one or more target genes. Additionally, CRISPR systems can be used in vivo (see, e.g., Chen H, Shi M, Gilam A, et al. Hemophilia A ameliorated in mice by CRISPR-based in vivo genome editing of human Factor VIII. Sci Rep. 2019; 9(1):16838; Hana S, Peterson M, McLaughlin H, et al. Highly efficient neuronal gene knockout in vivo by CRISPR-Cas9 via neonatal intracerebroventricular injection of AAV in mice. Gene Ther. 2021; 28(10-11):646-658; and Rosenblum D, Gutkin A, Kedmi R, et al. CRISPR-Cas9 genome editing using targeted lipid nanoparticles for cancer therapy. Sci Adv. 2020; 6(47):eabc9450).

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

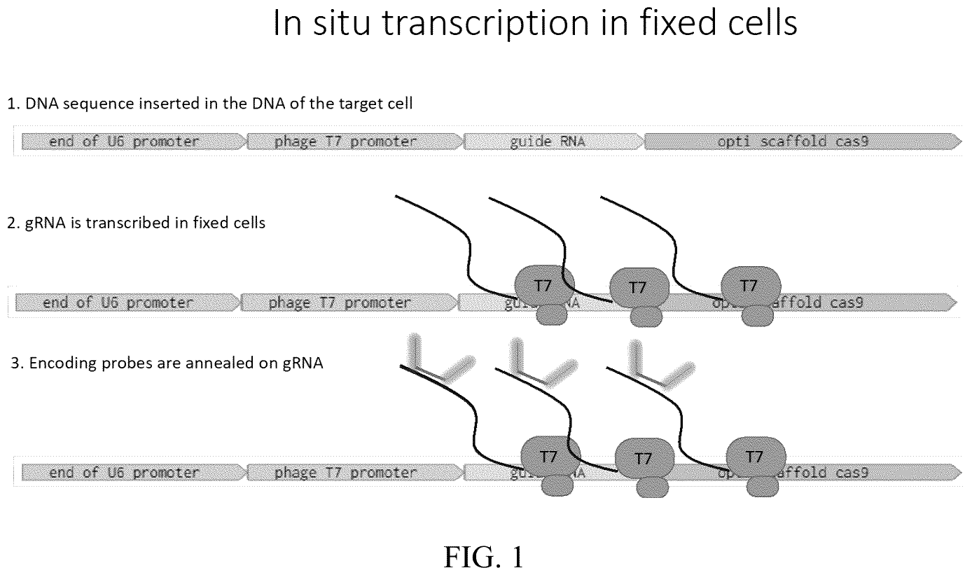

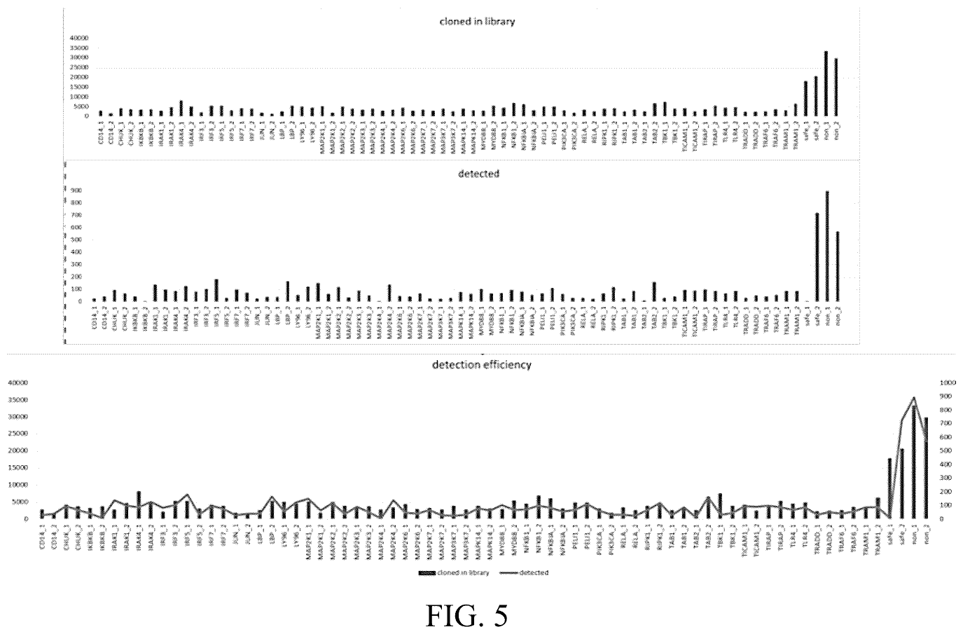

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically comprise a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

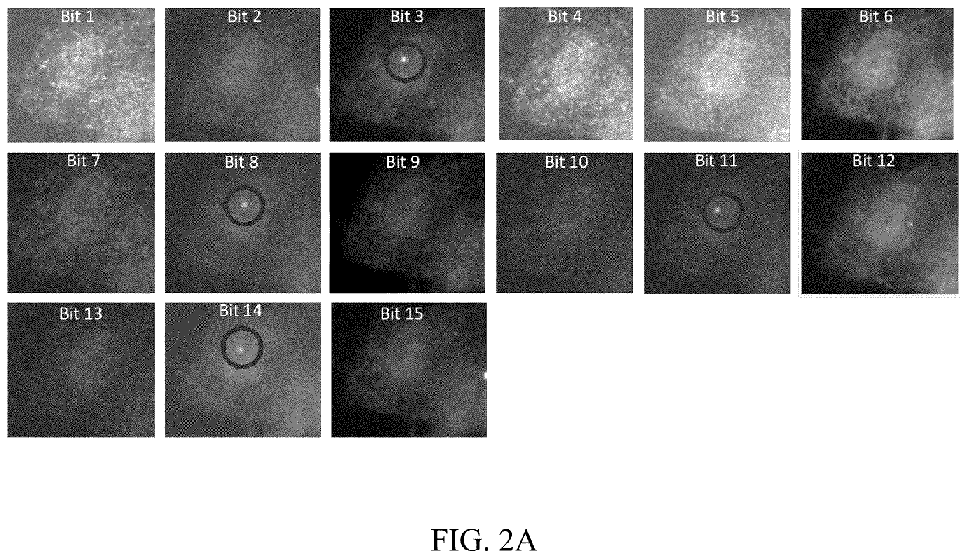

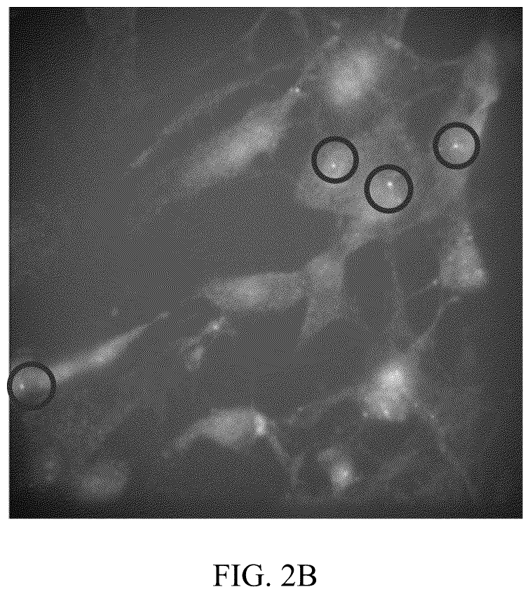

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at FIG. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the spacer sequence and tracr sequence, along the length of the shorter of the two sequences. Optimal alignment may be determined by any suitable alignment algorithm and may further account for secondary structures, such as self-complementarity within either the spacer sequence or tracr sequence. In some embodiments, the degree of complementarity between the tracr sequence and spacer sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A (from Gleditzsch et al. 2019) below shows several Cas polypeptides and the PAM sequence they recognize.

TABLE A
Example PAM Sequences

| | |
|---|---|
| Cas Protein | PAM Sequence |
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including LbCpf1 | |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, TAG, and |
| | CAC |
| Type I-C | NTTC |
| Type I-E | 5′-AAG-3′ |
| Type I-F | GG |

In a preferred embodiment, the CRISPR effector protein may recognize a 3′ PAM. In one example embodiment, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 10) or PKKKRKVEAS (SEQ ID NO: 11); the NLS from nucleoplasmin (e.g., the nucleoplasmin bipartite NLS with the sequence KRPAATKKAGQAKKKK (SEQ ID NO: 12)); the c-myc NLS having the amino acid sequence PAAKRVKLD (SEQ ID NO: 13) or RQRRNELKRSP (SEQ ID NO: 14); the hRNPA1 M9 NLS having the sequence NQSSNFGPMKGGNFGGRSSGPYGGGGQYFAKPRNQGGY (SEQ ID NO: 15); the sequence RMRIZFKNKGKDTAELRRRRVEVSVELRKAKKDEQILKRRNV (SEQ ID NO: 16) of the IBB domain from importin-alpha; the sequences VSRKRPRP (SEQ ID NO: 17) and PPKKARED (SEQ ID NO: 18) of the myoma T protein; the sequence PQPKKKPL (SEQ ID NO: 19) of human p53; the sequence SALIKKKKKMAP (SEQ ID NO: 20) of mouse c-abl IV; the sequences DRLRR (SEQ ID NO: 21) and PKQKKRK (SEQ ID NO: 22) of the influenza virus NS1; the sequence RKLKKKIKKL (SEQ ID NO: 23) of the Hepatitis virus delta antigen; the sequence REKKKFLKRR (SEQ ID NO: 24) of the mouse Mx1 protein; the sequence KRKGDEVDGVDEVAKKKSKK (SEQ ID NO: 25) of the human poly(ADP-ribose) polymerase; and the sequence RKCLQAGMNLEARKTKK (SEQ ID NO: 26) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

In certain embodiments, the CRISPR-Cas protein and a functional domain protein (described further herein) are delivered to the cell or expressed within the cell as separate proteins. In these embodiments, each of the CRISPR-Cas and functional domain protein can be provided with one or more NLSs as described herein. In certain embodiments, the CRISPR-Cas and functional domain protein are delivered to the cell or expressed with the cell as a fusion protein. In these embodiments one or both of the CRISPR-Cas and functional domain protein is provided with one or more NLSs. Where the functional domain protein is fused to an adaptor protein (such as MS2) as described above, the one or more NLS can be provided on the adaptor protein, provided that this does not interfere with aptamer binding. In particular embodiments, the one or more NLS sequences may also function as linker sequences between the functional domain protein and the CRISPR-Cas protein.

In certain embodiments, guides of the disclosure comprise specific binding sites (e.g., aptamers) for adapter proteins, which may be linked to or fused to a functional domain protein or catalytic domain thereof. When such a guide forms a CRISPR complex (e.g., CRISPR-Cas protein binding to guide and target), the adapter proteins bind and the functional domain protein or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the functional domain protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively, or additionally, the NES or NLS may be at the N terminus of component. In some examples, the Cas protein and optionally said functional domain protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### OMEGA Systems

In one example embodiment, the programmable nuclease to modify the one or more target genes is a transposon-encoded RNA-guided nuclease system, referred to herein as OMEGA (obligate mobile element-guided activity). See, e.g., Altae-Tran H, Kannan S, Demircioglu F E, et al. The widespread IS200/IS605 transposon family encodes diverse programmable RNA-guided endonucleases. Science. 2021; 374(6563):57-65. OMEGA systems include, but are not limited to IscB, IsrB, TnpB systems.

In some embodiments, the nucleic acid-guided nucleases herein may be an IscB protein (see, e.g., International patent application publication No. WO2022087494A1; and Altae-Tran H, et al. 2021). An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated. In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the nucleic acid-guided nucleases herein may be an IsrB (Insertion sequence RuvC-like OrfB) protein (see, e.g., International patent application publication No. WO2022087494A1; and Altae-Tran H, et al. 2021). IsrB refers to a group of shorter, ~350 aa IscB homologs that are also encoded in IS200/605 superfamily transposons. These proteins contain a PLMP domain and split RuvC but lack the HNH domain.

In some embodiments, the nucleic acid-guided nucleases herein may be a TnpB protein (see, e.g., International patent application publication No. WO2022159892A1; and Altae-Tran H, et al. 2021). TnpB is a putative endonuclease distantly related to IscB and thought to be the ancestor of Cas12, the type V CRISPR effector. The TnpB system comprises a TnpB polypeptide and a nucleic acid component capable of forming a complex with the TnpB polypeptide and directing the complex to a target polynucleotide. The TnpB systems and TnpB/nucleic acid component complexes may also be referred to herein as OMEGA (Obligate Mobile Element Guided Activity) systems or complexes, or Ω systems or complexes for short. TnpB systems are a distinct type of Ω system, which further include IscB, IsrB, and IshB systems. The nucleic acid component of Ω systems is structurally distinct from other RNA-guided nucleases, such as CRISPR-Cas systems, and may also be referred to as a ωRNA. In certain example embodiments, the TnpB systems are RNA-predominate, that is the nucleic acid component makes a larger contribution to the overall size of the TnpB complex relative to other RNA-guided nuclease systems such as CRISPR-Cas. Also, given the more minimal structural features of TnpB relative other known programmable nucleases such as CRISPR-Cas, the polynucleotide binding pocket is open and more accessible, which can facilitate greater access to and ability to manipulate, modify, edit, remove, or delete nucleotides at a target region on the bound polynucleotide.

Accordingly, it is contemplated within the scope of the present invention that OMEGA systems may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

Accordingly, it is contemplated within the scope of the present invention that Zn finger nucleases may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IIPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more-half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 27)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 28)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Accordingly, it is contemplated within the scope of the present invention that TALE nucleases may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

Accordingly, it is contemplated within the scope of the present invention that meganucleases may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### Other Genetic Modification Systems

A number of alternative gene modification systems have been developed that utilize the target specificity of a programmable nuclease but that modify or replace that nuclease activity with another functional activity. For example, programmable nucleases may be modified such that they cleave only a single strand as opposed to both strands of a target polynucleotide. Such “nickases” may then be paired with other functional domains such as reverse transcriptases, recombinases and non-LTR retrotransposon polypeptides to make genetic modifications that do not rely on creating double strand breaks. Similarly, programmable nucleases may also be modified to eliminate the nuclease activity altogether. These catalytically inactive or “dead” nucleases may then be combined with other functional domains like nucleotide deaminases, transposases, non-LTR retrotransposon polypeptides, methylases, deactylases, and acetylases, among other domains. The following provides further examples of gene modification systems that may be used in the context of the present invention. For ease of reference the gene modifications systems that follow will be discussed in the context of using CRISPR-Cas as the programmable nuclease system, but it is contemplated within the scope of this invention that the nickase or dead Cas versions described below could be replaced by a comparable nickase or dead nuclease variant of other programmable nucleases/systems such as OMEGA systems, Zn finger nucleases, TALE nucleases, and meganucleases.

### DNA and RNA Base Editing

In one example embodiment, the perturbation comprises administering a DNA or RNA base editing system to either decrease expression of one or more genes or increase expression of one or more genes. In one example embodiment, a catalytically inactive Cas protein is connected or fused to a nucleotide deaminase. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems. Accordingly, in one example embodiment, the base editing system edits the target gene to reduce or eliminate its expression or to increase its expression.

In one example embodiment, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In one example embodiment, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Example DNA Base Editing Modifications to Increase or Decrease Expression of Target Genes

In one embodiment, a DNA base editing system may be configured to make one or more base edits in a non-coding region of one or more genes that result in decreased expression of the one or more genes or increase expression of one or more genes.

DNA Base-Editing Modifications that Decrease Expression by Targeting Non-Coding Regions

In one embodiment, a DNA base editing system may be configured to make one or more base edits in a non-coding region of one or more genes that result in decreased expression of the one or more genes. In one embodiment, the one or more base edits introduce mutations in an enhancer region controlling expression of one or more genes to prevent or disrupt the binding of transcription factors such that transcription initiation and gene expression are blocked or reduced. In one example embodiment, the one or more base edits introduce mutations in a promoter region controlling expression of one or more genes to prevent or disrupt the binding of transcription factors and/or RNA polymerase such that transcription initiation and gene expression are blocked or reduced. In one example embodiment, the base editor configured to one or more base edits that introduce a new silence region or modify and strengthen an existing silence region controlling expression of one or more genes leading to the recruitment of transcriptional repressors that block or decrease gene expression. In another embodiment, the base editor is configured to make one or more base edits that disrupt one or more insulator sequences controlling expression of one or more genes such that nearby silencer elements or repressive chromatin structures are able to decrease gene expression. In one embodiment, a DNA base editing system may be configured to make one or more base edits in a non-coding region of one or more genes that result in increased expression of the one or more genes.

DNA Base-Editing Modifications that Increase Expression by Targeting Non-Coding Regions

In one embodiment, a DNA base editing system may be configured to make one or more base edits in a non-coding region of one or more genes that result in increased expression of the one or more genes. In one embodiment, the base editing system is configured to introduce one or more base edits in one or more enhancer regions controlling expression of one or more genes such that binding of transcription factors or other regulatory proteins is increased or strengthened and gene expression increased. In another embodiment, the base editing system is configured to introduce one or more base edits in one or more promoters controlling expression of one or more genes such that binding of transcription factors and/or RNA polymerase is increased or strengthened and gene expression is increased. In another embodiment, the base editing system is configured to introduce one or more base edits that disrupt or remove one or more silencer elements that control expression of one or more genes, such that binding of transcriptional repressors is prevented or weakened and gene expression is increased. In another embodiment, the base editing system is configured to introduce or strengthen insulator sequences controlling expression of the one or more genes thereby reducing the influence of nearby silencer elements or repressive chromatin structures such that gene expression is increased.

DNA Base-Editing Modifications that Decrease Expression by Targeting Coding Regions

In one embodiment, a DNA base editing system may be configured to make one or more base edits in a coding region of one or more genes that result in decreased expression of the one or more genes. In one embodiment, the one or more base edits result in a frame-shift mutation leading to introduction of a premature stop codon and the production of non-functional truncated gene products, or the triggering of nonsense-mediated mRNA decay (NMD), thereby resulting in reduced expression or gene product activity. In another embodiment, the one or more base edits result in introduction of a premature stop codon within the coding region resulting in production of truncated non-functional proteins or the triggering of NMD. thereby resulting in reduced gene expression or gene product activity. In another embodiment, the one or more base edits target specific functional domains within the coding region to create mutations that impair the function of the gene product. While this approach may not directly decrease gene expression, it can lead to the production of non-functional proteins, effectively resulting in a loss-of-function effect. In another embodiment, the one or more base edits may introduce mutations in the coding region at exon-intron boundaries or splice sites leading to aberrant splicing, production of non-function proteins or triggering NMD and thereby reducing gene expression or activity of a resulting gene product. In another embodiment, the one or more base edits may target regulatory elements within the coding regions that affect gene expression, such as internal ribosome entry sites (IRES). One or more modifications may be made at these regulatory elements to reduce gene expression. In another embodiment, the one or more base edits may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to active a proteins function. Accordingly, modifications that introduce inhibitory PTMs or remove activating PTMs may be made to decrease protein function, stability, and/or degradation.

DNA Base-Editing Modifications that Increase Expression by Targeting Coding Regions

In one embodiment, the base editing system are configured such that one or more base edits are mode in a coding region of the one or more genes such that expression of the one or more genes is increased. In one embodiment, the one or more base edits comprise removing or disrupting inhibitor sequences, such as IRESs or upstream open reading frames (uORFs), which can negatively affect expression. In one embodiment, the one or more base edits may comprise introducing specific mutations within the coding region that can potentially improve protein stability, folding, or resistance to degradation. While this does not directly increase gene expression, it can lead to higher protein levels and enhanced function. In one embodiment, the one or more base edits may comprise removal or disruption of a sequence encoding an inhibitory PTM site, removal or disruption of one or more ubiquitination sites, or introduction of PTM sites that stabilize or enhance protein function. In one embodiment, the one or more base edits may comprise mutations or modifications within the coding region that improve catalytic activity, binding affinity, or other functional properties of the protein. This approach does not directly increase gene expression but can result in an overall increase in the functional output of the gene product.

### Example RNA Base Editing Modifications to Increase or Decrease Expression of Target Genes

RNA base editors enable targeted RNA editing without modifying the underlying DNA sequence and may be useful where more temporal control of gene expression is desired.

In one embodiment, a RNA base editing system is used to introduce one or more base edits to one or more RNA molecules transcribed from one or more genes. such that expression or activity of the gene product is reduced. In one embodiment, the one or more base edits introduce a frame-shift mutation leading to introduction of a premature stop code resulting in production of a truncated protein or triggering NMD, both which lead to decreased gene expression. In another embodiment, the one or more base edits introduce splice sites or splice regulatory elements that lead to aberrant splicing and production of non-functional proteins or mRNA that is degraded through NMD, thereby decreasing gene expression. In one embodiment, the one or more base edits target specific functional domains of the gene product encoded within the mRNA that impair the function of the gene product. While this approach may not directly decrease translation of the mRNA, it leads to the production of non-functional gene product or gene products with decreased function, effectively achieving a loss-of-function effect. In one embodiment, the one or more base edits modify regulatory elements within the mRNA. Some mRNAs have regulatory elements that can affect gene expression, such as upstream reading frames (uORFs) or IRESs and disrupting these elements may reduce gene expression. In one embodiment, the one or more base edits target translation initiation or elongation by introducing mutations in the mRNA's 5′ untranslated (5′UTR), 3′ untranslated region (3′UTR), or within the coding sequence, affecting translation initiation or elongation and resulting in decreased production of a gene product.

In one embodiment, a RNA base editing system is used to introduce one or more base edits to one or more RNA molecules transcribed from one or more genes. such that expression or activity of the gene product is increased. In one embodiment, the one or more base edits are used to change suboptimal codons to more frequently used codes (while maintaining the same amino acid sequence) in the coding region of the mRNA, leading to improved translation efficiency and gene product production. In one embodiment, the one or more base edits remove inhibitor sequences in the mRNA. Some mRNAs contain regulatory elements, such as uORFs and IRESs, that inhibit gene expression. The one or more base edits may be used to disrupt or remove these inhibitor sequences thereby increasing gene product production. In one embodiment, the one or more base edits may be used to modify regulatory elements within the mRNA, such as mRNA stability elements, microRNA binding sites, or RNA binding protein sites may enhance mRNA stability, translation efficiency, or prevent degradation, leading to increased gene expression. In one embodiment, the one or more base edits may introduce one or more mutations in the 5′ UTR or the 3′UTR that enhance translation initiation or elongation, resulting in increased gene product production. In one embodiment, the one or more base edits may be used to introduce specific point mutations or modifications within the coding region of the mRNA using RNA based editors that can potentially improve the catalytic activity, binding affinity, or other functional properties of the gene product. While this approach may not directly increase mRNA translation it can result in an overall increase of the functional output of the gene product.

### ARCUS Base Editing

In one example embodiment, a target gene is modified with an ARCUS base editing system. Exemplary methods for using ARCUS can be found in U.S. Pat. No. 10,851,358, US Publication No. 2020-0239544, and WIPO Publication No. 2020/206231 which are incorporated herein by reference.

In certain embodiments, the ARCUS base editing system comprises a nuclease, derived from I-CreI endonuclease (hereinafter, an “ARC Nuclease”) with a recognition sequence for one or more genes. In certain embodiments, the nuclease is a homing endonuclease or meganuclease as described in the section titled “Meganucleases”. In certain embodiments, the ARC Nuclease is an engineered meganuclease prepared to recognize a target gene or transcription factor, or region of a target gene or transcription factor. In certain embodiments, the ARC Nuclease comprises a single-component protein containing both a site-specific DNA recognition interface and endonuclease activity. The combination of both substrate-recognition and catalytic motifs into a single protein have been shown to allow for both viral and non-viral delivery modalities (see, e.g., Gorsuch et al. (2022). Targeting the hepatitis B cccdna with a sequence-specific arcus nuclease to eliminate hepatitis B virus in vivo. Molecular Therapy, 30(9), 2909-2922. doi.org/10.1016/j.ymthe.2022.05.013).

In one example embodiment, the ARC nuclease is configured to decrease the expression of the one or more genes or transcription factors or increase the expression of the one or more genes or transcription factors. In an example embodiment, the ARC nuclease scans a region of a target gene for the target site. For example, the ARCUS nuclease looks for a polynucleotide or region within one or more open reading frames of one or more genes. After binding to the target site, the DNA sequence is cut, created a sticky 4-base 3′ overhang wherein the cut target site is repaired via HDR or NHEJ. As discussed previously, NHEJ can result in insertions, deletions, substitutions, or otherwise a frameshift mutation that can interfere with gene expression. In one example embodiment, the interference with gene expression results in the decreased expression of one or more genes or increased expression of one or more genes. HDR or NHEJ methods for repaired joining, an optionally, specific templates that could be utilized, are described in the respective sections titled “HDR Template Based Editing” and “NHEJ-Based Editing”. In certain embodiments, an additional template may prevent off-site insertions or deletions.

### Prime Editors

In one example embodiment, the perturbation comprises administering a prime editing system to either decrease expression of one or more genes or increase the expression of one or more genes. Prime editing systems comprise a programable nuclease (e.g. Cas), most often a nickase, linked to a reverse transcriptase domain and a guide molecule (prime editing guide pegRNA), which comprises a target-specific spacer, a primer binding site, and RT template. See e.g., Anzalone et al. 2019. Nature. 576: 149-157; and International Patent Application Publication No. WO2022150790A2. In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

Prime editing systems can also be used in tandem such that, the two pegRNAs template the synthesis of complementary DNA flaps on opposing strands of genomic DNA, which replace the endogenous DNA sequence between the PE-induced nick sites. See, e.g., Anzalone A V, Gao X D, Podracky C J, et al. Programmable deletion, replacement, integration and inversion of large DNA sequences with twin prime editing. Nat Biotechnol. 2022; 40(5):731-740. Thus, use of two pegRNAs allows for larger insertions or deletions because of the two overlapping 3′ flaps created by the two nicked sites. In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

### Recombinase-Mediated Modifications

Prime editing and twinPE systems can also be further combined with site-specific recombinases, such as integrases, to facilitate even larger insertions, substitutions and deletions. See e.g., WO 2021/138469; Anzalone A V, Gao X D, Podracky C J, et al. Programmable deletion, replacement, integration and inversion of large DNA sequences with twin prime editing. Nat Biotechnol. 2022; 40(5):731-740; Yarnall et al., Nat Biotechnol (2022). doi.org/10.1038/s41587-022-01527-4, which is incorporated by reference as if expressed in its entirety herein. The prime editing system is used to insert a recombinase recognition site at the desire site of modification and an integrase facilitates the insertion of a donor sequence from a donor template. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

In example embodiments, the recombinase of the present invention is a serine integrase. In example embodiments, serine integrases specifically recombine when recognizing the two attachment sites specific for the integrase. In example embodiments, the heterologous sites are referred to as attP and attB, however, these terms refer to the specific sequences recognized by the specific integrase and do not refer to a single consensus sequence. Serine integrases mediate site-specific recombination between short recognition sites located in phage genomes and bacterial chromosomes, respectively, the attachment site of phage (attP) and attachment site of bacteria (attB) (i.e., the target sites of the integrase), to form the hybrid attachment sites attL and attR. Unlike Cre and Flp recombinases that catalyze reversible site-specific recombination reactions, serine integrases are unidirectional and catalyze only attP and attB recombination without RDF or Xis accessory proteins. Thus, in the absence of any accessory factors, integrase is unidirectional. In addition, DNA substrates identified by serine integrases (attP and attB) are relatively short (30-50 bp) and have a minimal length of approximately 34-40 base pairs (bp) (Groth A C et al., Proc. Natl. Acad. Sci. USA 97, 5995-6000 (2000)). The compatibility of distinct DNA topological structures is also quite different from recognition of DNA by Hin recombinase or Tn3 resolvase. Serine integrases recognize DNA substrates specifically, not at random, but can facilitate recombination at sequences with partial identity with wild-type recombination sites, termed pseudo attachment sites (either pseudo attP or pseudo attB). A “pseudo-recombination site” is a DNA sequence recognized by a recombinase enzyme such that the recognition site differs in one or more base pairs from the wild-type recombinase recognition sequence and/or is present as an endogenous sequence in a genome that differs from the genome where the wild-type recognition sequence for the recombinase resides. “Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site. Specific attB and attP sequences for use in the present invention include all wildtype sequences as well as pseudo attB and attP sequences.

Recombination sites used in the present methods include those recognized by unidirectional, site-directed recombinases (e.g., integrases). Non-limiting examples of serine integrases and recombination sites applicable to the present invention include φC31 integrase, Bxb1, φBT1 integrase, A118, TP901-1, and R4 and the corresponding recombination sites for each (see, e.g., Groth, A. C. and Calos, M. P. (2004) J. Mol. Biol. 335, 667-678; Lei, et al., FEBS Lett. 2018 April; 592(8):1389-1399; Singh, et al., Attachment Site Selection and Identity in Bxb1 Serine Integrase-Mediated Site-Specific Recombination, PLoS Genet. 2013 May; 9(5):e1003490; and Gupta, et al., Nucleic Acids Res. 2007 May; 35(10): 3407-3419). Additional serine recombinases and recombination sites may be any of those disclosed in US 20180346934A1 and US 2010/0190178. In certain embodiments, a functional domain of the serine integrase is used.

In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### Example Prime Editing Modifications for Decreasing or Increasing Expression of Target Genes

Prime Editing systems may be used to introduce insertions, deletions, or substitutions (modifications) that control expression of one or more genes. The modifications may be made in a non-coding region that controls expression of the one or more target genes, in a coding region encoding a gene expression product (e.g., a polypeptide), or both. Example modifications are described in further detail below. As a threshold matter, Primer Editing systems are capable of making all 4 base edits (A, T, C, G) and thus can be used to make all of the same DNA base edits described above in the Base Editor section. The following examples focus on additional insertions, deletions and substitutions that may be made using prime editors beyond single base edits using standard prime editors (PE), twinPE, or PE/twinPE in combination with a recombinase, which for purposes of the following example modification sections will be referred to collectively as prime editors.

Prime Editing Modifications that Decrease Expression by Targeting Non-Coding Regions

In one example embodiment, the prime editing system is configured to introduce a deletion, insertion, or mutation in one or more non-coding controlling expression of one or more genes such the expression of the one or more genes is reduced. In one embodiment, the one or more modifications remove, modify, or disrupt an enhancer such that binding of transcription factors or other regulatory proteins controlling expression is disrupted thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the prime editing system is configured to introduce a silencer element into the non-coding region leading to the recruitment of transcriptional repressors that block or decrease gene expression. In one embodiment, the prime editor is configured to modify or replace an existing silencer element such that the silencing function of the silencer element is increased relative to an unmodified silencer sequence. In another embodiment, the prime editor is configured to disrupt or replace one or more insulator sequences such that nearby silencer elements or repressive chromatin structures can decrease gene expression.

Prime Editing Modifications that Increase Expression by Targeting Non-Coding Regions

In another embodiment, the prime editor is configured to introduce one or more enhancer regions controlling expression of one or more genes such that binding of transcription factors or other regulatory proteins is increased or strengthened and gene expression is increased. In another embodiment, the primer editor is configured to introduce one or more modifications in one or more promoters controlling expression of one or more genes such that binding of transcription factors or RNA polymerase is increased or strengthened and gene expression is increased. In another embodiment, the prime editor is configured to introduce insertions/deletions/substitution that disrupt or remove one or more silence elements thereby preventing binding of transcriptional repressors and increasing gene expression. In another embodiment, the prime editor is configured to introduce or strengthen insulator sequences, thereby reducing the influence of nearby silencer elements or repressive chromatin structures such that gene expression is increased.

Prime Editing Modifications that Decrease Expression by Targeting Coding Regions

In one embodiment, the prime editor is configured such that one or more modifications (e.g., insertions, deletions, substitutions) are mode in a coding region of the one or more genes such that expression of the one or more genes is reduced. In on embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to active a proteins function. Accordingly, modifications that introduce inhibitory PTMs or remove activating PTMs may be made to decrease protein function, stability, and/or degradation.

Prime Editing Modifications that Increase Expression by Targeting Coding Regions

In one embodiment, the programmable nuclease and donor template are configured such that one or more modifications (e.g., insertions, deletions, substitutions) are made in a coding region of one or more genes such that expression of the one or more genes is increased. In one embodiment, the one or more modifications comprise removing inhibitors sequences, such as IRESs or upstream open reading frames (uORFs), which can negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modification may comprise mutations or modifications within the coding region that improve catalytic activity, binding affinity, or other functional properties of the protein. This approach does not directly increase gene expression but can result in an overall increase in the functional output of the gene product. In another embodiment, prime editing and/or twinPE are used in combination with a recombinase to insert an additional functional copy of one or more genes.

### CRISPR Associated Transposase (CAST) Systems

In one example embodiment, the perturbation comprises administering a gene editing system to either decrease expression of one or more target genes or increase expression of one or more target, wherein the gene editing system configured to modify the one or more target genes or transcription factors is a CAST system.

CAST systems are able to insert DNA sequences at a target site in a DNA molecule without relying on host cell repair machinery. CAST systems can be Class 1 or Class 2 CAST systems. For example, a Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference. Suitable hybrid systems have also been described such as those described in Tou et al. bioRxiv 2022.01.07.475005, doi.org/10.1101/2022.01.07.475005, which is incorporated herein by reference. Additional CAST systems are disclosed, e.g., in WO 2020/131862 to Zhang et al and WO/2021/257997 to Zhang et al. (CAST-12k); WO 2021/087394 to Zhang et al. and WO/2022/147321 to Zhang et al. (CAST-lb); WO 2022/076820 to Zhang et al. (CAST-if); WO/2022/150651 to Zhang et al. (minimal Tn7-like CAST systems), all of which are incorporated herein by reference.

The CAST system may comprise a Cas linked to a transposase subunit to achieve RNA-guided DNA-transposition, optionally linked to a guide molecule. In some embodiments, the Cas may be catalytically inactive (e.g., Type I, IV, or Type V systems). Transposases suitable for the CAST system may be of any variety generally derived Tn7-like transposons (e.g. non-limiting examples, including TnsA, TnsB, TnsC, or TniQ). Guide molecules can guide the catalytically inactive Cas and a Tn7 or Tn7-like subunit to a target site to direct insertion of a donor at the target site.

CAST systems may require combinatorial transposases for efficient deposition. For example, TnsA is an endonuclease that cleaves the 5′-ends of the transposon and interacts with TnsB, TnsC, and DNA. TnsB is a recombinase capable of cleaving the 3′-end of the transposon. In some CAST systems with these components, the interaction between TnsA and TnsB achieves catalysis. In another instance, TnsC can direct TnsA and TnsB to the insertion site. In another instance, TniQ and DNA can be recognized by TnsC and enable the Cas complex to achieve insertion at the site.

In one example embodiment, the CAST system can be used to insert or replace a sequence into one or more target genes. In example embodiments, the insertion or replacement results in an inactive target gene or less active form of the target gene. In one example embodiment, a CAST system is used to replace all or a portion of an enhancer controlling the target gene expression. In an example embodiment, the enhancer controls the expression of one or more target genes.

CAST systems may be used to introduce one or more modifications (insertions, deletions, substitutions) that modify expression of one or more genes. The modifications may be made in a non-coding region that controls expression of the one or more target genes, in a coding region encoding a gene expression product (e.g., a polypeptide), or both. Example modifications are described in further detail below.

CAST Modifications that Decrease Expression by Targeting Non-Coding Regions

In one example embodiment, the CAST system is configured to introduce a deletion, insertion, or mutation in one or more non-coding regions controlling expression of one or more genes such the expression of the one or more genes is reduced. In one embodiment, the one or more modifications remove, modify, or disrupt an enhancer such that binding of transcription factors or other regulatory proteins controlling expression is disrupted, thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the one or more modifications comprise introduction of a silencer element into the non-coding region leading to the recruitment of transcriptional repressors that block or decrease gene expression. In one embodiment, the one or more modifications comprise modifying or replacing an existing silencer element such that the silencing function of the silencer element is increased relative to an unmodified silencer sequence. In another embodiment, the one or more modifications comprise disrupting or replacing one or more insulator sequences such that nearby silencer elements or repressive chromatin structures can decrease gene expression.

CAST Modifications that Decrease Expression by Targeting Non-Coding Regions

In another embodiment, the CAST system is configured to introduce one or more modifications controlling expression of one or more genes such that binding of transcription factors or other regulatory proteins is increased or strengthened and gene expression is increased. In one embodiment, the one or more modifications comprise one or more modifications in one or more promoters such that binding of transcription factors or RNA polymerase is increased or strengthened and gene expression is increased. In another embodiment, the one or more modifications comprise introduction of insertions/deletions/substitution that disrupt or remove one or more silence elements thereby preventing binding of transcriptional repressors and increasing gene expression. In another embodiment, the one or more modifications comprise the introduction of insulator sequences, either new insulator sequences or modified versions of pre-existing insulator sequences, thereby reducing the influence of nearby silencer elements or repressive chromatin structures such that gene expression is increased.

CAST Modifications that Decrease Expression by Targeting Coding Regions

In one embodiment, the CAST systems are configured such that one or more modifications (e.g., insertions, deletions, substitutions) are made in a coding region of the one or more genes such that expression of the one or more genes is reduced. In on embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to active a proteins function. Accordingly, modifications that introduce inhibitory PTMs or remove activating PTMs may be made to decrease protein function, stability, and/or degradation.

CAST Modifications that Increase Expression by Targeting Coding Regions

In one embodiment, the CAST system are configured such that one or more modifications (e.g., insertions, deletions, substitutions) are made in a coding region of one or more genes such that expression of the one or more genes is increased. In one embodiment, the one or more modifications comprise removing inhibitors sequences, such as IRESs or upstream open reading frames (uORFs), that negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modifications may comprise mutations or modifications within the coding region that improve catalytic activity, binding affinity, or other functional properties of the protein. This approach does not directly increase gene expression but can result in an overall increase in the functional output of the gene product. In another embodiment, the CAST system is used to insert an additional functional copy of one or more genes.

### Non-LTR Retrotransposon Systems

In one example embodiment, the perturbation comprises administering a Non-LTR Retrotransposon system to either decrease expression of one or more target genes or increase expression of one or more target genes.

The Non-LTR retrotransposon system may comprise one or more components of a retrotransposon, e.g., a non-LTR retrotransposon. Native or wild-type non-LTR retrotransposons encode the protein machinery necessary for their self-mobilization. The non-LTR retrotransposon element comprises a DNA element integrated into a host genome. The DNA element may encode one or two open reading frames (ORFs). For example, the R2 element of Bombyx mori encodes a single ORF containing reverse transcriptase (RT) activity and a restriction enzyme-like (REL) domain. Li elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. In an example embodiment, the RNA-transposase complex nicks the genome and the 3′ end of the nicked DNA serves as a primer to allow the reverse transcription of the transposon RNA into cDNA. The transposase proteins may then integrate the cDNA into the genome.

Elements of these systems may be engineered to work within the context of the invention. For example, a non-LTR retrotransposon polypeptide may be fused to a programmable nuclease. The binding elements that allow a non-LTR retrotransposon polypeptide to bind to the native retrotransposon DNA element, may be engineered into a donor construct to facilitate entry of a donor polynucleotide sequence into a target polypeptide.

In certain embodiments, the protein component of the non-LTR retrotransposon may be connected to or otherwise engineered to form a complex with a programmable nuclease, e.g., a Cas polypeptide. The retrotransposon RNA may be engineered to encode a donor polynucleotide sequence. Thus, in certain example embodiments, the Cas polypeptide, via formation of a CRISPR-Cas complex with a guide sequence, directs the retrotransposon complex (i.e., the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence. Example non-LTR retrotransposon systems are disclosed in WO 2021/102042, WO 2022/173830, which are incorporated herein by reference.

Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805, which are incorporated by reference herein in their entireties.

Examples of the non-LTR retrotransposon polypeptides also include R2 from Clonorchis sinensis, or Zonotrichia albicollis. Example non-LTR retrotransposon polypeptides and binding components (5′ and 3′ UTRs) that may be used in the context of the invention are listed in Table 1 along with codon optimized variants of the non-LTR retrotransposons for expression in eukaryotic cells.

A non-LTR retrotransposon may comprise multiple retrotransposon polypeptides or polynucleotides encoding same. In some embodiments, the retrotransposon polypeptides may form a complex. For example, a non-LTR retrotransposon is a dimer, e.g., comprising two retrotransposon polypeptides forming a dimer. The dimer subunits may be connected or form a tandem fusion. A Cas protein or polypeptide may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a Cas protein or polypeptide.

The retrotransposon polypeptides may be enzymes or variants thereof. In some examples, a retrotransposon polypeptide may be a reverse transcriptase, a nuclease, a nickase, a transposase, nucleic acid polymerase, ligase, or a combination thereof. In one example, a retrotransposon polypeptide is a reverse transcriptase. In another example, a retrotransposon polypeptide is a nuclease. In another example, a retrotransposon polypeptide is nickase. In a particular example, a non-LTR retrotransposon comprises a first retrotransposon polypeptide and a second retrotransposon polypeptide, wherein the second retrotransposon polypeptide comprises nuclease or nickase activity. In certain cases, a retrotransposon polypeptide may comprise an inactive enzyme. For example, a retrotransposon polypeptide may comprise a nuclease domain that is inactivated. Such inactivated domain may serve as a nucleic acid binding domain.

The retrotransposon polypeptides may comprise one or more modifications to, for example, enhance specificity or efficiency of donor polynucleotide recognition, target-primed template recognition (TPTR), and/or reduce or eliminate homing function. The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol2 promoter, a pol3 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In some embodiments, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end, the 3′ end, or a location in between.

In some embodiments, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Donor Constructs

The systems may comprise one or more donor constructs comprising one or more donor polynucleotide sequences for insertion into a target polynucleotide. The donor construct comprises one or more binding elements. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for interacting to the retrotransposon polypeptide.

In certain example embodiments, the donor construct comprises a 5′ binding element and a 3′ binding element with a donor polynucleotide sequence located between the 5′ and 3′ prime binding element.

A donor polynucleotide may be any type of polynucleotides, including, but not limited to, a gene, a gene fragment, a non-coding polynucleotide, a regulatory polynucleotide, a synthetic polynucleotide, etc.

A target polynucleotide may comprise a protospacer adjacent motif (PAM) sequence. An example of the PAM sequence is AT.

The donor construct may further comprise one or more processing element. The processing element is an element that may be added to ensure accurate processing and incorporation of the donor polynucleotide sequence by the fusion proteins disclosed herein. Example processing elements include, but are not limited to, LRNA processing elements (e.g. GGCTCGTTGGGAGGTCCCGGGTTGAAATCCCGGACGAGCCCG (SEQ ID NO: 29)), human 28s processing elements (e.g. TAGCCAAATGCCTCGTCATCTAATTAGTGACGCGCATGAATGGATGAACGAGATTCC CACTGTCCCTACCTACTATCCAGCGAAACCACAGCCAAGGGAA (SEQ ID NO: 30)), and natural retrotransposon processing elements such as R2 processing elements from Bombyx mori (e.g. tagccaaatgcctcgtcatctaattagtgacgcgcatgaatggattaacgagattcccactgtccctatctactatctagcgaaaccacagcca agggaacgggcttgggagaatcagcggggaa (SEQ ID NO: 31)).

The donor construct may comprise one or more homology sequence. A homology sequence is a sequence that shares or complete or partial homology with a target sequence at the site the targeted site of insertion. The homology sequence may be located on the 5′ end, ′3 end, or on both the 5′ and 3′ end of the donor construct. In certain example embodiments, the homology sequence is only located on the 5′ end of the donor construct. In certain example embodiments, the homology sequence is located only on the 3′ end of the donor construct. In certain example embodiments, the location of the homology sequence may depend on whether the site-specific nuclease is being directed to create a nick or cut 5′ or 3′ of the targeted insertion site, e.g. a 5′ homology sequence on the donor construct may be used when the site specific nuclease creates a nick or cut 5′ of the targeted insertion site and a 3′ homology sequence may be used when the site-specific nuclease is configured to create a nick or cut 3′ of the targeted insertion site. In certain example embodiments, the homology sequence is included on both the 5′ and 3′ ends of the donor construct regardless of whether the site-specific nuclease creates a nick or cut 5′ or 3′ of the targeted insertion site. In certain example embodiments the donor construct may comprise in a 5′ to 3′, a binding element, and the donor sequence. In certain example embodiments the donor construct may comprise in a 5′ to 3′ direction a homology sequence, a binding element, and the donor sequence. In certain example embodiments the donor construct may comprise in a 5′ to 3′ direction a homology sequence, a first binding element, the donor sequence, and second binding element. In certain example embodiments, the donor construct may comprise in a 5′ to 3′ direction a first homology sequence, a first binding element, the donor sequence, and a second homology sequence. In certain example embodiments the donor construct may comprise, in a 5′ to 3′ direction, a first homology sequence, a first binding element, the donor sequence, a second binding element, and a second homology sequence. In certain example embodiments, the donor construct may comprise, in a 5′ to 3′ direction, the donor sequence and a binding element. In certain example embodiments, the donor construct may comprise, in a 5′ to 3′ direction, the donor sequence, a binding element, and a homology sequence. A processing element may be further incorporated 3′ of the donor sequence in any of the above donor construct configurations.

The homology sequence may have at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 bases of homology to the target DNA. In certain example embodiments, the homology sequence may have between 8, 9, 10, 11, 12, 13, 14, 15, 16, 17, 18, 19, 20, 21, 22, 23, 24, or 25 base pairs of homology to the target sequence. In embodiments, with a homology sequence on both the 5′ and 3′ end of the donor construct, the size of the homology may be the same or different on each end. In some examples, the homology sequence comprises from 1 to 30, from 4 to 10, or from 10 to 25 nucleotides. For example, the homology sequence comprises from 4 to 10 nucleotides. For example, the homology sequence comprises from 10 to 25 nucleotides. For example, the homology sequence comprises 1 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

The donor polynucleotides may be inserted to the upstream or downstream of the PAM sequence of a target polynucleotide. For example, the donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

In a strand of a polynucleotide, anything towards the 5′ end of a reference point is “upstream” of that point, and anything towards the 3′ end of a reference point is “downstream” of that point. A location upstream of a PAM sequence refers to a location at the 5′ side of the PAM sequence on the PAM-containing strand of the target sequence. A location downstream of a PAM sequence refers to a location at the 3′ side of the PAM sequence on the PAM-containing strand of the target sequence.

The compositions and systems herein may be used to insert a donor polynucleotide with desired orientation. For example, appropriate homology sequence may be selected to control the orientation of insertion on the 5′ or 3′ strand of the target sequence.

The donor polynucleotide comprises a homology sequence of a region of the target sequence. The homology sequence may share at least 50%, at least 60%, at least 70%, at least 80%, at least 90%, at least 95%, or 100% sequence identity with the region of the target sequence. In an example, the homology sequence shares 100% sequence identity with the region of the target sequence.

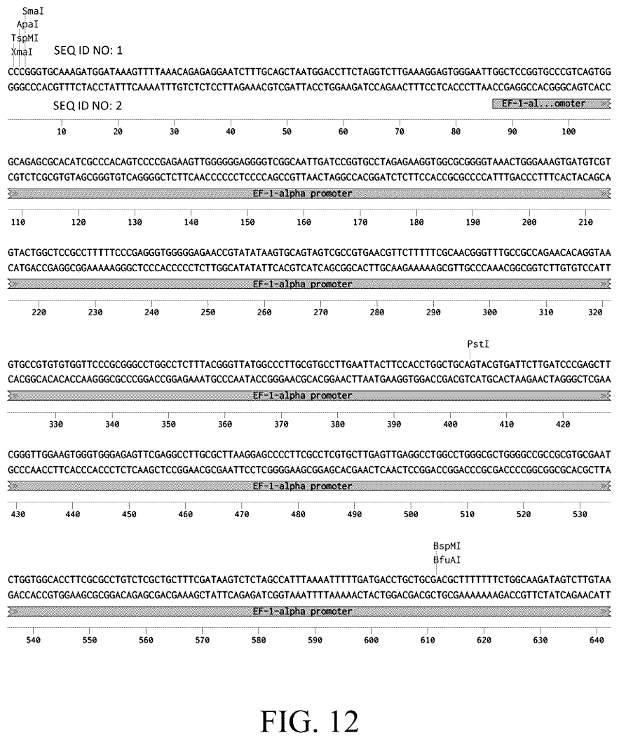

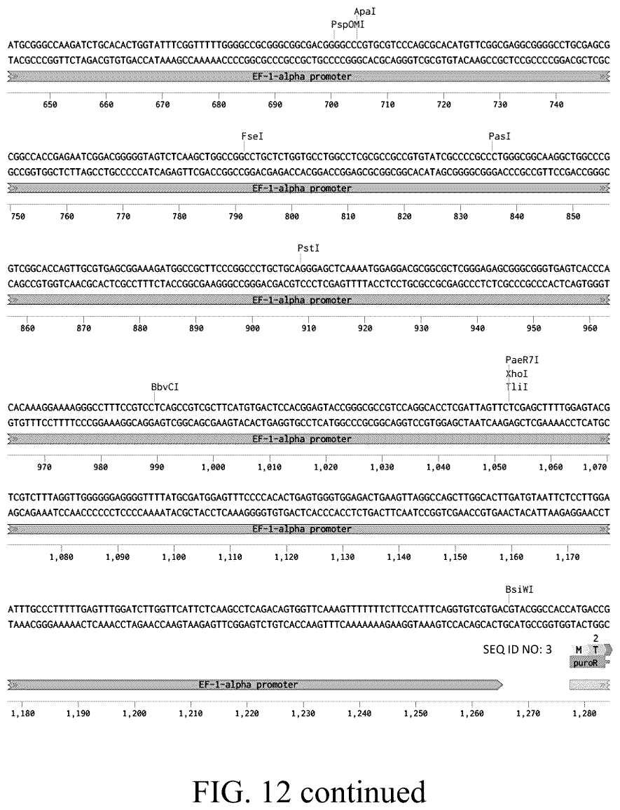

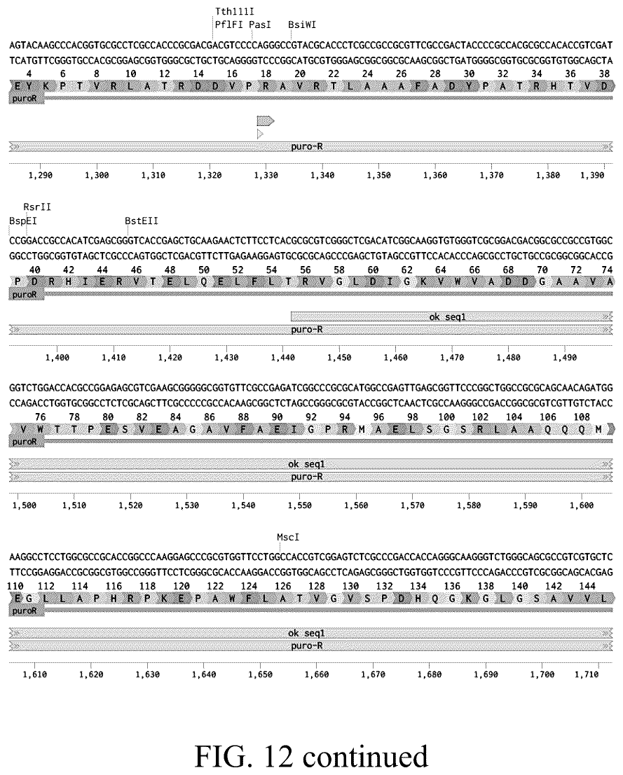

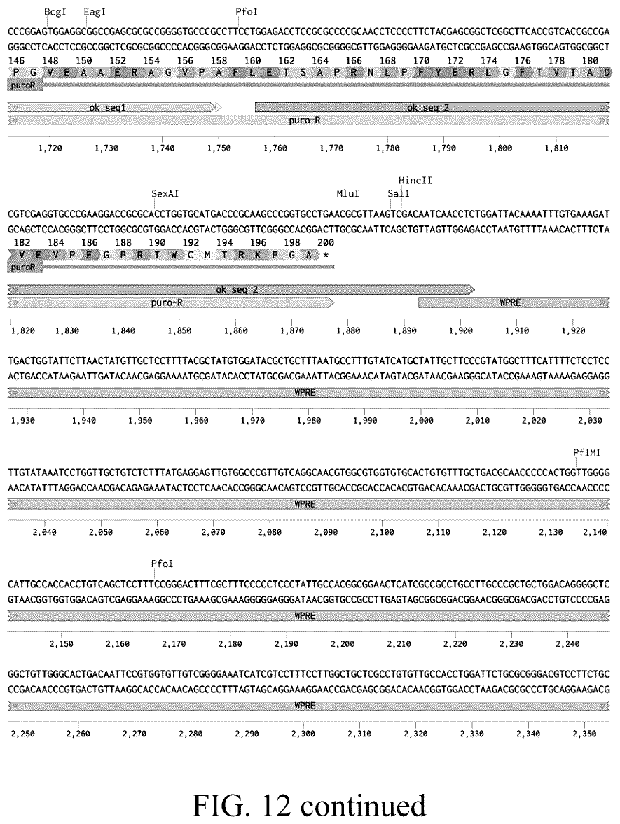

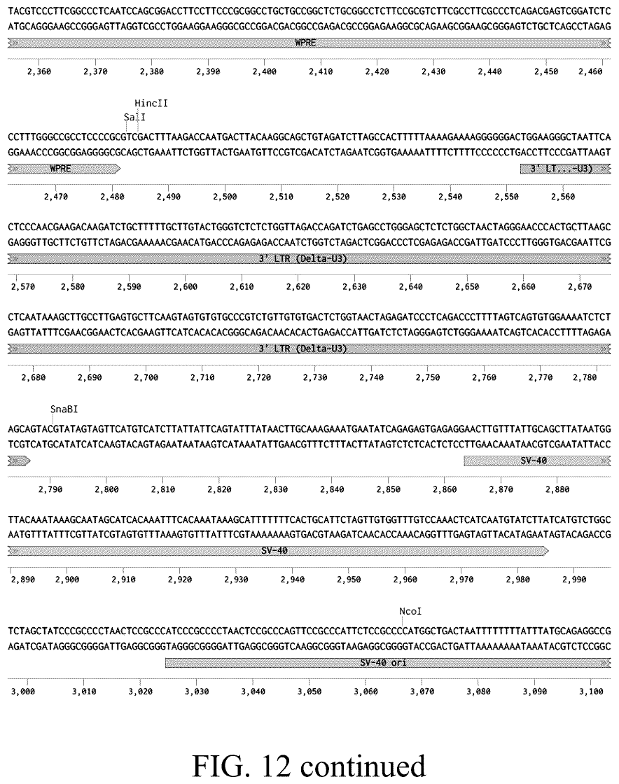

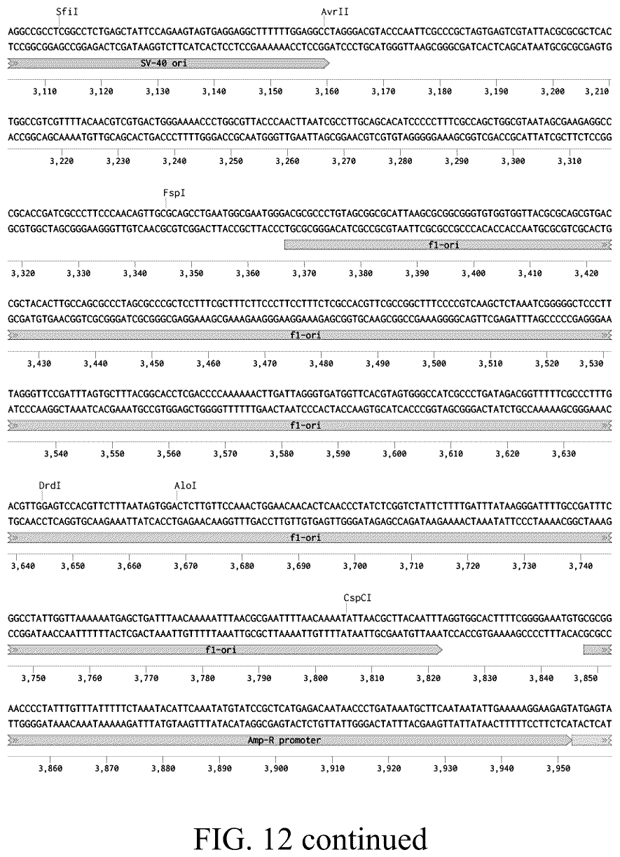

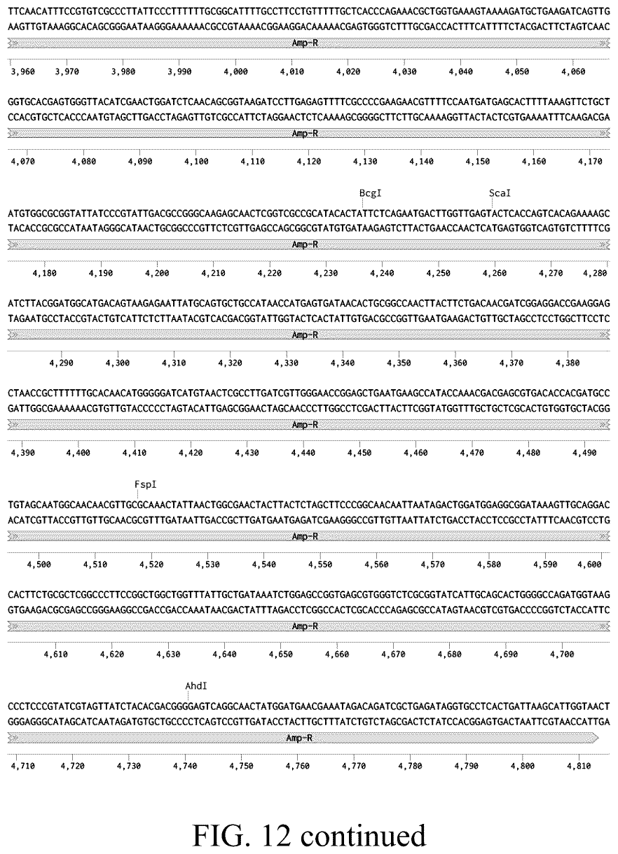

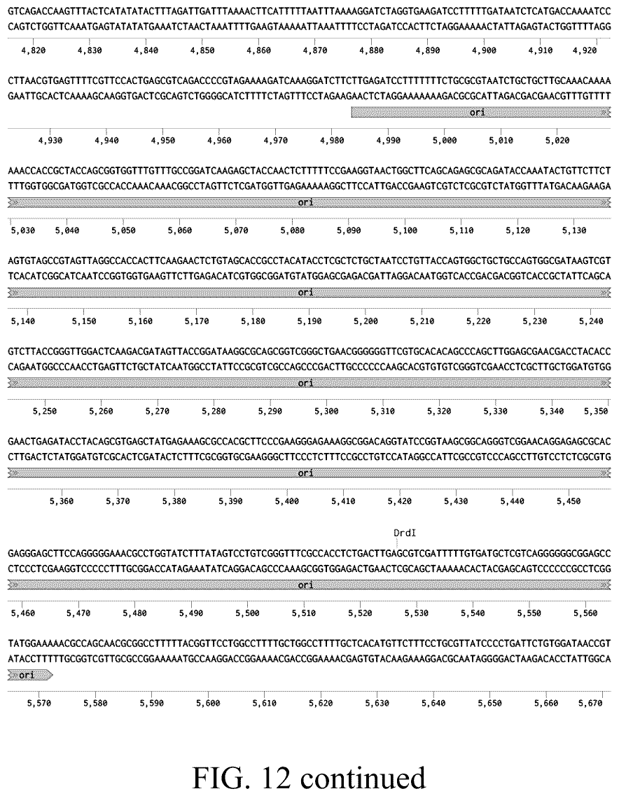

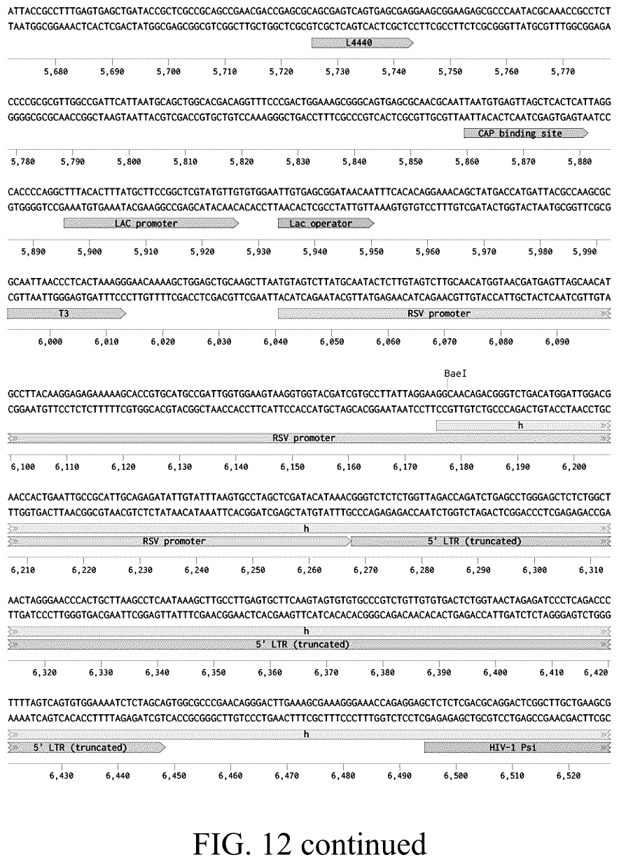

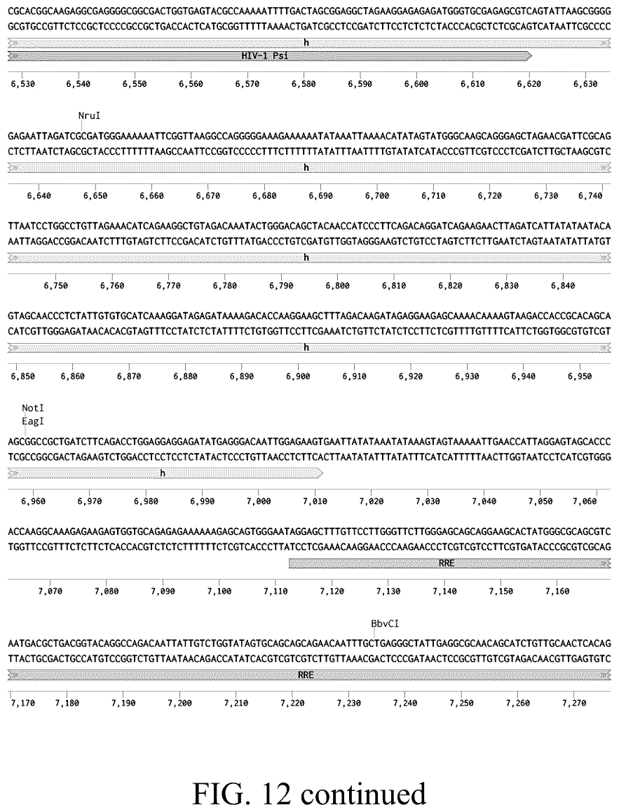

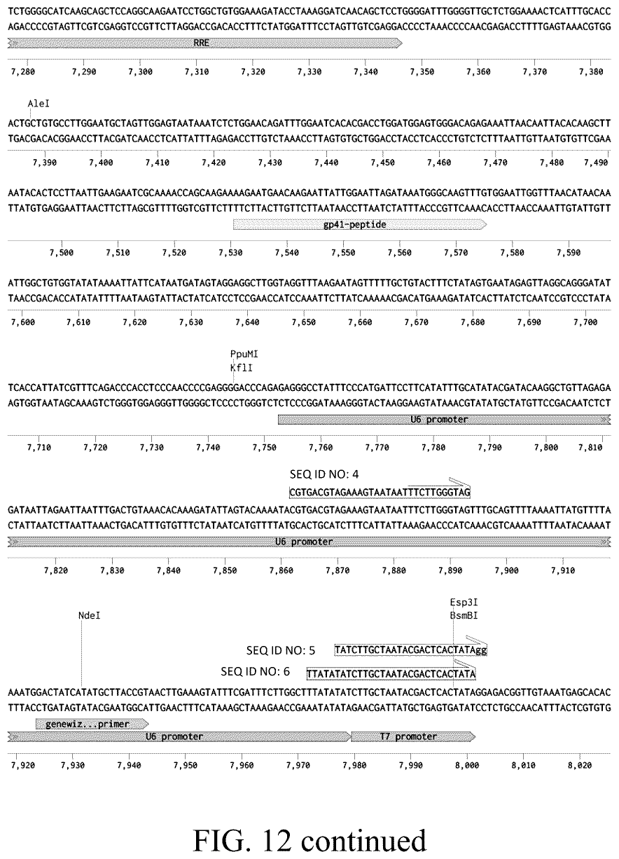

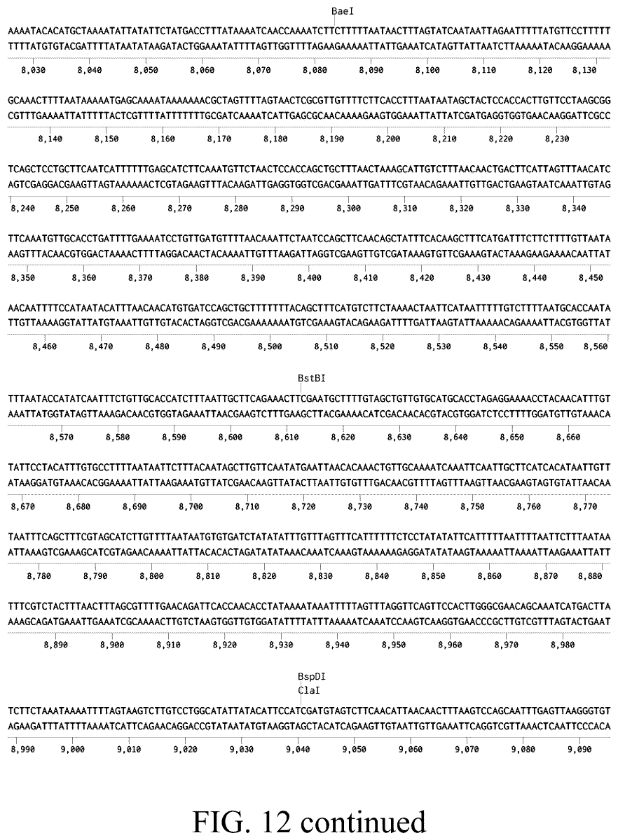

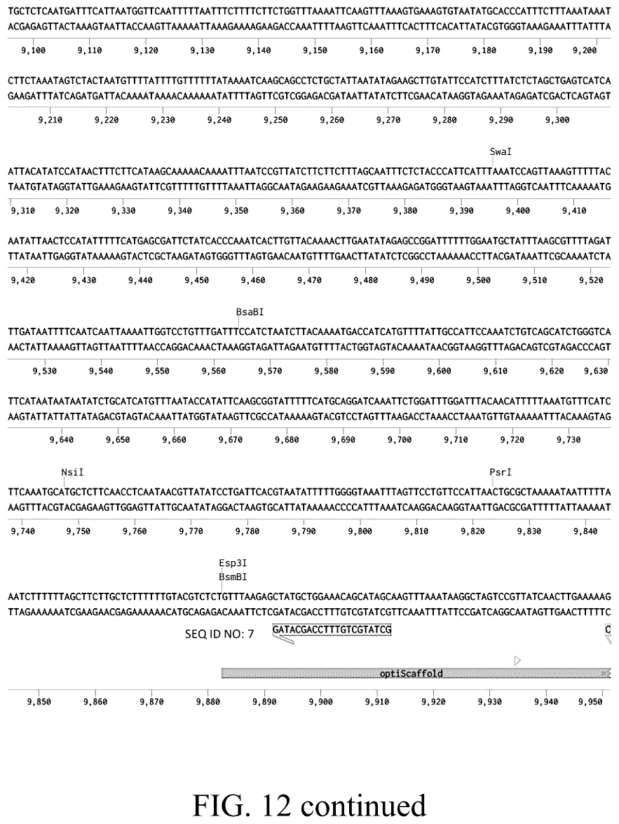

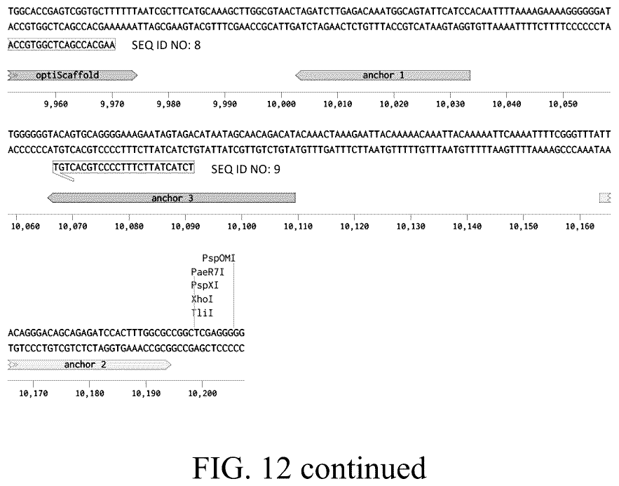

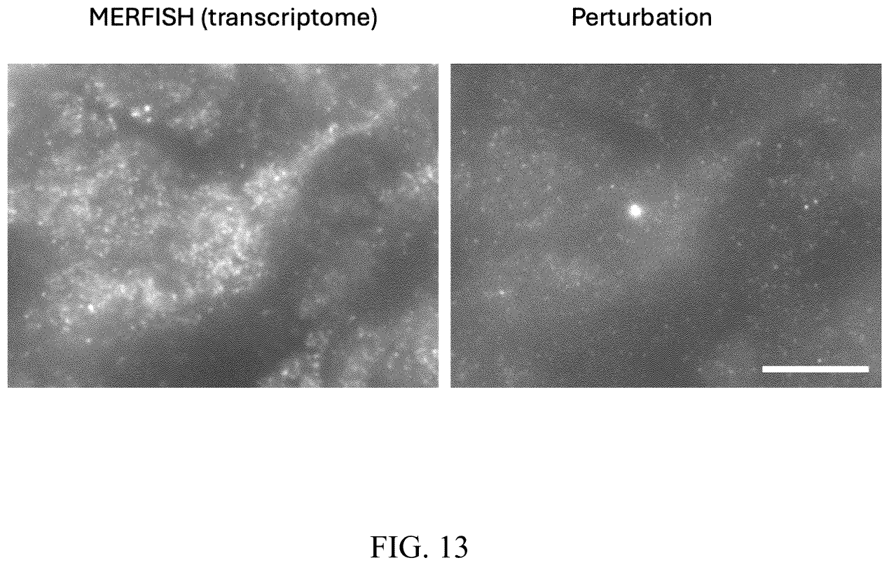

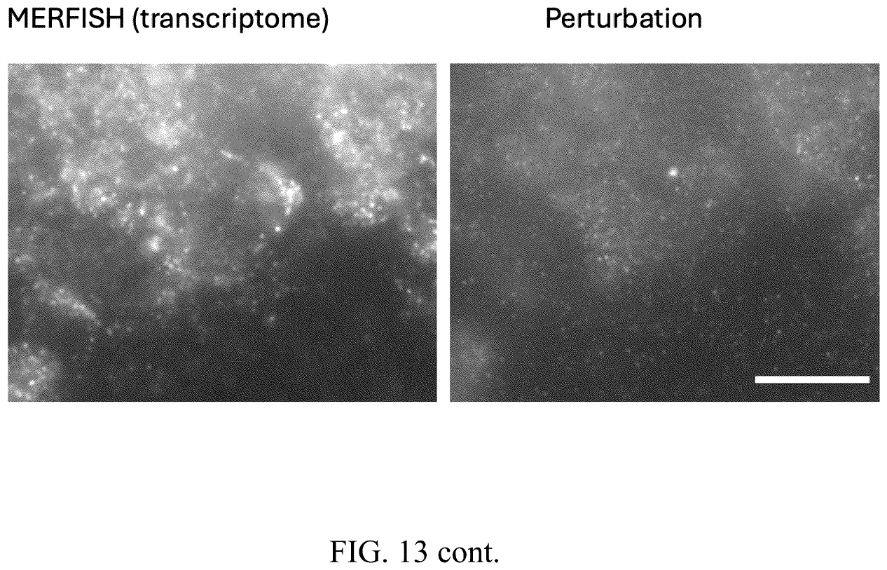

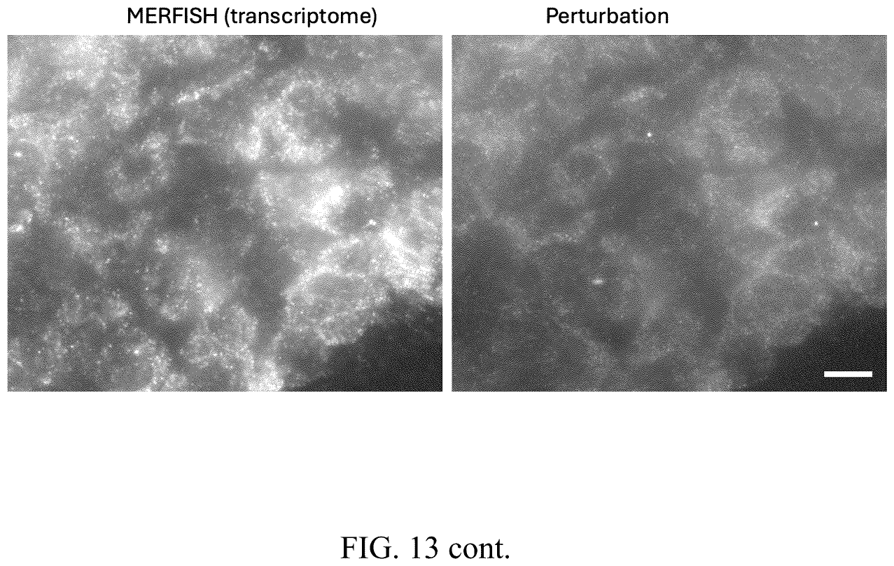

In some embodiments, the donor polynucleotide may be inserted to the strand on the target sequence that contains the PAM (e.g., the PAM sequence of the site-specific nuclease such as Cas). In such cases, the donor polynucleotide may comprise a homology sequence of a region on the PAM containing strand of the target sequence. Such region may comprise the PAM sequence. The region may be at the 3′ side of the cleavage site of the site-specific nuclease. In some examples, the homology sequence may comprise from 4 to 10, or from 10 to 25 nucleotides in length. An example of such homology sequence may be of the “h1” region shown in FIG. 12.

In some embodiments, the donor polynucleotide may be inserted to the strand on the target sequence that binds to the guide, e.g., the strand that contains a guide-binding sequence. In such cases, the donor polynucleotide may comprise a homology sequence of a region that comprises at least a portion of the guide-binding sequence. In some cases, the region may comprise the entire guide-binding sequence. Such region may further comprise a sequence at the 3′ side of the guide-binding sequence. For example, the region may comprise from 5 to 15 nucleotides, e.g., 5, 6, 7, 8, 9, 10, 11, 12, 13, 14, 15 nucleotides from the 3′ side of the guide-binding sequence. In some cases, the region may be adjacent to the R-loop of the guide. For example, in the cases where the guide forms a RNA-DNA duplex with the guide-binding sequence, the region comprises a sequence at the 3′ side from the RNA-DNA duplex, e.g., from 5 to from 5 to 15 nucleotides, e.g., 5, 6, 7, 8, 9, 10, 11, 12, 13, 14, 15 nucleotides from the 3′ side from the RNA-DNA duplex. An example of such homology sequence may be of the “h2” region shown in FIG. 12.

In some examples, the homology sequence is of a region on the target sequence at 3′ side of a PAM-containing strand. In certain examples, the homology sequence is of a region on the target sequence 10 nucleotides from 3′ side of a RNA-DNA duplex formed by a guide molecule and a target sequence. For example, the guide molecule forms a RNA-DNA duplex with the target sequence, and the homology sequence is of a region on the target sequence 5 to 15 nucleotides from 3′ side of the RNA-DNA duplex. In some embodiments, the donor polynucleotide is inserted to a region on the target sequence that is 3′ side of a PAM-containing strand. In some cases, the donor polynucleotide is inserted to a region on the target sequence that is 3′ side of a sequence complementary to the guide molecule.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of the corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 5 bases to 50 kb in length, e.g., from 50 to 40 kb, from 100 and 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, from 2800 bases to 3000 bases, from 2900 bases to 3100 bases, from 3000 bases to 3200 bases, from 3100 bases to 3300 bases, from 3200 bases to 3400 bases, from 3300 bases to 3500 bases, from 3400 bases to 3600 bases, from 3500 bases to 3700 bases, from 3600 bases to 3800 bases, from 3700 bases to 3900 bases, from 3800 bases to 4000 bases, from 3900 bases to 4100 bases, from 4000 bases to 4200 bases, from 4100 bases to 4300 bases, from 4200 bases to 4400 bases, from 4300 bases to 4500 bases, from 4400 bases to 4600 bases, from 4500 bases to 4700 bases, from 4600 bases to 4800 bases, from 4700 bases to 4900 bases, or from 4800 bases to 5000 bases in length.

### Example Non-LTR Retrotransposon-Mediated Modifications for Decreasing or Increasing Expression of Target Genes

Non-LTR retrotransposon systems may be used to introduce one or more modifications (insertions, deletions, substitutions) that modify expression of one or more genes. The modifications may be made in a non-coding region that controls expression of the one or more target genes, in a coding region encoding a gene expression product (e.g., a polypeptide), or both. Example modifications are described in further detail below.

Non-LTR Modification that Decrease Expression by Targeting Non-Coding Regions

In one example embodiment, the non-LTR retrotransposon system is configured to introduce a deletion, insertion, or mutation in one or more non-coding regions controlling expression of one or more genes such the expression of the one or more genes is reduced. In one embodiment, the one or more modifications remove, modify, or disrupt an enhancer such that binding of transcription factors or other regulatory proteins controlling expression is disrupted, thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the one or more modifications comprise introduction of a silencer element into the non-coding region leading to the recruitment of transcriptional repressors that block or decrease gene expression. In one embodiment, the one or more modifications comprise modifying or replacing an existing silencer element such that the silencing function of the silencer element is increased relative to an unmodified silencer sequence. In another embodiment, the one or more modifications comprise disrupting or replacing one or more insulator sequences such that nearby silencer elements or repressive chromatin structures can decrease gene expression.

Non-LTR Modifications that Increase Expression by Targeting Non-Coding Regions

In another embodiment, the non-LTR retrotransposon system is configured to introduce one or more modifications controlling expression of one or more genes such that binding of transcription factors or other regulatory proteins is increased or strengthened and gene expression is increased. In one embodiment, the one or more modifications comprise one or more modifications in one or more promoters such that binding of transcription factors or RNA polymerase is increased or strengthened and gene expression is increased. In another embodiment, the one or more modifications comprise introduction of insertions/deletions/substitution that disrupt or remove one or more silence elements thereby preventing binding of transcriptional repressors and increasing gene expression. In another embodiment, the one or more modifications comprise the introduction of insulator sequences, either new insulator sequences or modified versions of pre-existing insulator sequences, thereby reducing the influence of nearby silencer elements or repressive chromatin structures such that gene expression is increased.

Non-LTR Modifications that Decrease Expression by Targeting Coding Regions

In one embodiment, the non-LTR retrotransposon systems are configured such that one or more modifications (e.g., insertions, deletions, substitutions) are made in a coding region of the one or more genes such that expression of the one or more genes is reduced. In on embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to active a proteins function. Accordingly, modifications that introduce inhibitory PTMs or remove activating PTMs may be made to decrease protein function, stability, and/or degradation.

Non-LTR Modifications that Increase Expression by Targeting Coding Regions

In one embodiment, the non-LTR retrotransposon system is configured such that one or more modifications (e.g. insertions, deletions, substitutions) are made in a coding region of the one or more genes such that expression of the one or more genes is increased. In one embodiment, the one or more modifications comprise removing inhibitors sequences, such as IRESs or upstream open reading frames (uORFs), that negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modifications may comprise mutations or modifications within the coding region that improve catalytic activity, binding affinity, or other functional properties of the protein. This approach does not directly increase gene expression but can result in an overall increase in the functional output of the gene product. In another embodiment, the non-LTR retrotransposon system is used to insert an additional functional copy of one or more genes.

### Epigenetic Editing

In one example embodiment, the perturbation comprises an epigenetic modifier or modification system to either decrease expression of one or more genes or increase expression of one or more genes. or a combination thereof. In one example embodiment, the one or more agents is an epigenetic modifier polypeptide comprising a DNA binding domain linked to or otherwise capable of associating with an epigenetic modification domain such that binding of the DNA binding domain at target sequence on genomic DNA (e.g., chromatin) results in one or more epigenetic modifications by the epigenetic modification domain that increases or decreases expression of the one or more polypeptides disclosed herein. As used herein, “linked to or otherwise capable of associating with” refers to a fusion protein or a recruitment domain or an adaptor protein, such as an aptamer (e.g., MS2) or an epitope tag. The recruitment domain or an adaptor protein can be linked to an epigenetic modification domain or the DNA binding domain (e.g., an adaptor for an aptamer). The epigenetic modification domain can be linked to an antibody specific for an epitope tag fused to the DNA binding domain. An aptamer can be linked to a guide sequence.

In example embodiments, the DNA binding domain is a programmable DNA binding protein linked to or otherwise capable of associating with an epigenetic modification domain. Programmable DNA binding proteins for modifying the epigenome include, but are not limited to CRISPR systems, OMEGA systems, transcription activator-like effectors (TALEs), Zn finger proteins and meganucleases (see, e.g., Thakore P I, Black J B, Hilton I B, Gersbach C A. Editing the epigenome: technologies for programmable transcription and epigenetic modulation. Nat Methods. 2016; 13(2):127-137; and described further herein). In example embodiments, the DNA binding domain is a nuclease-deficient RNA-guided DNA endonuclease enzyme or a nuclease-deficient endonuclease enzyme. In example embodiments, a CRISPR system having an inactivated nuclease activity (e.g., dCas) is used as the DNA binding domain.

In example embodiments, the epigenetic modification domain is a functional domain and includes, but is not limited to a histone methyltransferase (HMT) domain, histone demethylase domain, histone acetyltransferase (HAT) domain, histone deacetylation (HDAC) domain, DNA methyltransferase domain, DNA demethylation domain, histone phosphorylation domain (e.g., serine and threonine, or tyrosine), histone ubiquitylation domain, histone sumoylation domain, histone ADP ribosylation domain, histone proline isomerization domain, histone biotinylation domain, histone citrullination domain (see, e.g., Epigenetics, Second Edition, 2015, Edited by C. David Allis; Marie-Laure Caparros; Thomas Jenuwein; Danny Reinberg; Associate Editor Monika Lachlan; Dawson M A, Kouzarides T. Cancer epigenetics: from mechanism to therapy. Cell. 2012; 150(1):12-27; Syding L A, Nickl P, Kasparek P, Sedlacek R. CRISPR/Cas9 Epigenome Editing Potential for Rare Imprinting Diseases: A Review. Cells. 2020; 9(4):993; and Zhang Y. Transcriptional regulation by histone ubiquitination and deubiquitination. Genes Dev. 2003; 17(22):2733-2740). Example epigenetic modification domains can be obtained from, but are not limited to chromatin modifying enzymes, such as, DNA methyltransferases (e.g., DNMT1, DNMT3a and DNMT3b), TET1, TET2, thymine-DNA glycosylase (TDG), GCN5-related N-acetyltransferases family (GNAT), MYST family proteins (e.g., MOZ and MORF), and CBP/p300 family proteins (e.g., CBP, p300), Class I HDACs (e.g., HDAC 1-3 and HDAC8), Class II HDACs (e.g., HDAC 4-7 and HDAC 9-10), Class III HDACs (e.g., sirtuins), HDAC11, SET domain containing methyltransferases (e.g., SET7/9 (KMT7, NCBI Entrez Gene: 80854), KMT5A (SET8), MMSET, EZH2, and MLL family members), DOT1L, LSD1, Jumonji demethylases (e.g., KDM5A (JARID1A), KDM5C (JARIDIC), and KDM6A (UTX)), kinases (e.g., Haspin, VRK1, PKCα, PKCβ, PIM1, IKKα, Rsk2, PKB/Akt, Aurora B, MSK1/2, JNK1, MLTKα, PRK1, Chk1, Dlk/ZIP, PKCδ, MST1, AMPK, JAK2, Abl, BMK1, CaMK, S6K1, SIK1), Ubp8, ubiquitin C-terminal hydrolases (UCH), the ubiquitin-specific processing proteases (UBP), and poly(ADP-ribose) polymerase 1 (PARP-1). See, also, U.S. Pat. No. 11,001,829B2 for additional domains.

In example embodiments, histone acetylation is targeted to a target sequence using a CRISPR system (see, e.g., Hilton I B, et al. Epigenome editing by a CRISPR-Cas9-based acetyltransferase activates genes from promoters and enhancers. Nat Biotechnol. 2015). In example embodiments, histone deacetylation is targeted to a target sequence (see, e.g., Cong et al., 2012; and Konermann S, et al. Optical control of mammalian endogenous transcription and epigenetic states. Nature. 2013; 500:472-476). In example embodiments, histone methylation is targeted to a target sequence (see, e.g., Snowden A W, Gregory P D, Case C C, Pabo C O. Gene-specific targeting of H3K9 methylation is sufficient for initiating repression in vivo. Curr Biol. 2002; 12:2159-2166; and Cano-Rodriguez D, Gjaltema R A, Jilderda L J, et al. Writing of H3K4Me3 overcomes epigenetic silencing in a sustained but context-dependent manner. Nat Commun. 2016; 7:12284). In example embodiments, histone demethylation is targeted to a target sequence (see, e.g., Kearns N A, Pham H, Tabak B, et al. Functional annotation of native enhancers with a Cas9-histone demethylase fusion. Nat Methods. 2015; 12(5):401-403). In example embodiments, histone phosphorylation is targeted to a target sequence (see, e.g., Li J, Mahata B, Escobar M, et al. Programmable human histone phosphorylation and gene activation using a CRISPR/Cas9-based chromatin kinase. Nat Commun. 2021; 12(1):896). In example embodiments, DNA methylation is targeted to a target sequence (see, e.g., Rivenbark A G, et al. Epigenetic reprogramming of cancer cells via targeted DNA methylation. Epigenetics. 2012; 7:350-360; Siddique A N, et al. Targeted methylation and gene silencing of VEGF-A in human cells by using a designed Dnmt3a-Dnmt3L single-chain fusion protein with increased DNA methylation activity. J Mol Biol. 2013; 425:479-491; Bernstein D L, Le Lay J E, Ruano E G, Kaestner K H. TALE-mediated epigenetic suppression of CDKN2A increases replication in human fibroblasts. J Clin Invest. 2015; 125:1998-2006; Liu X S, Wu H, Ji X, et al. Editing DNA Methylation in the Mammalian Genome. Cell. 2016; 167(1):233-247.e17; Stepper P, Kungulovski G, Jurkowska R Z, et al. Efficient targeted DNA methylation with chimeric dCas9-Dnmt3a-Dnmt3L methyltransferase. Nucleic Acids Res. 2017; 45(4):1703-1713; and Pflueger C., Tan D., Swain T., Nguyen T., Pflueger J., Nefzger C., Polo J. M., Ford E., Lister R. A modular dCas9-SunTag DNMT3A epigenome editing system overcomes pervasive off-target activity of direct fusion dCas9-DNMT3A constructs. Genome Res. 2018; 28:1193-1206). In example embodiments, DNA demethylation is targeted to a target sequence using a CRISPR system (see, e.g., TET1, see Xu et al, Cell Discov. 2016 May 3; 2: 16009; Choudhury et al, Oncotarget. 2016 Jul. 19; 7(29):46545-46556; and Kang J G, Park J S, Ko J H, Kim Y S. Regulation of gene expression by altered promoter methylation using a CRISPR/Cas9-mediated epigenetic editing system. Sci Rep. 2019; 9(1):11960). In example embodiments, DNA demethylation is targeted to a target sequence (see, e.g., TDG, see, Gregory D J, Zhang Y, Kobzik L, Fedulov A V. Specific transcriptional enhancement of inducible nitric oxide synthase by targeted promoter demethylation. Epigenetics. 2013; 8:1205-1212).

Example epigenetic modification domains can be obtained from, but are not limited to transcription activators, such as, VP64 (see, e.g., Ji Q, et al. Engineered zinc-finger transcription factors activate OCT4 (POU5F1), SOX2, KLF4, c-MYC (MYC) and miR302/367. Nucleic Acids Res. 2014; 42:6158-6167; Perez-Pinera P, et al. Synergistic and tunable human gene activation by combinations of synthetic transcription factors. Nat Methods. 2013; 10:239-242; Farzadfard F, Perli S D, Lu T K. Tunable and multifunctional eukaryotic transcription factors based on CRISPR/Cas. ACS Synth Biol. 2013; 2:604-613; Black J B, Adler A F, Wang H G, et al. Targeted Epigenetic Remodeling of Endogenous Loci by CRISPR/Cas9-Based Transcriptional Activators Directly Converts Fibroblasts to Neuronal Cells. Cell Stem Cell. 2016; 19(3):406-414; and Maeder M L, Linder S J, Cascio V M, Fu Y, Ho Q H, Joung J K. CRISPR RNA-guided activation of endogenous human genes. Nat Methods. 2013; 10(10):977-979), p65 (see, e.g., Liu P Q, et al. Regulation of an endogenous locus using a panel of designed zinc finger proteins targeted to accessible chromatin regions. Activation of vascular endothelial growth factor A. J Biol Chem. 2001; 276:11323-11334; and Konermann S, et al. Genome-scale transcriptional activation by an engineered CRISPR-Cas9 complex. Nature. 2015; 517:583-588), HSF1, and RTA (see, e.g., Chavez A, et al. Highly efficient Cas9-mediated transcriptional programming. Nat Methods. 2015; 12:326-328); KRAB (see, e.g., Beerli R R, Segal D J, Dreier B, Barbas C F., 3rd Toward controlling gene expression at will: specific regulation of the erbB-2/HER-2 promoter by using polydactyl zinc finger proteins constructed from modular building blocks. Proc Natl Acad Sci USA. 1998; 95:14628-14633; Cong L, Zhou R, Kuo Y C, Cunniff M, Zhang F. Comprehensive interrogation of natural TALE DNA-binding modules and transcriptional repressor domains. Nat Commun. 2012; 3:968; Gilbert L A, et al. CRISPR-mediated modular RNA-guided regulation of transcription in eukaryotes. Cell. 2013; 154:442-451; and Yeo N C, Chavez A, Lance-Byrne A, et al. An enhanced CRISPR repressor for targeted mammalian gene regulation. Nat Methods. 2018; 15(8):611-616).

In example embodiments, the epigenetic modification domain linked to a DNA binding domain recruits an epigenetic modification protein to a target sequence. In example embodiments, a transcriptional activator recruits an epigenetic modification protein to a target sequence. For example, VP64 can recruit DNA demethylation, increased H3K27ac and H3K4me. In example embodiments, a transcriptional repressor protein recruits an epigenetic modification protein to a target sequence. For example, KRAB can recruit increased H3K9me3 (see, e.g., Thakore P I, D'Ippolito A M, Song L, et al. Highly specific epigenome editing by CRISPR-Cas9 repressors for silencing of distal regulatory elements. Nat Methods. 2015; 12(12):1143-1149). In an example embodiment, methyl-binding proteins linked to a DNA binding domain, such as MBD1, MBD2, MBD3, and MeCP2 recruits an epigenetic modification protein to a target sequence. In an example embodiment, Mi2/NuRD, Sin3A, or Co-REST recruit HDACs to a target sequence.

In example embodiments, the epigenetic modification domain can be a eukaryotic or prokaryotic (e.g., bacteria or Archaea) protein. In example embodiments, the eukaryotic protein can be a mammalian, insect, plant, or yeast protein and is not limited to human proteins (e.g., a yeast, insect, plant chromatin modifying protein, such as yeast HATs, HDACs, methyltransferases, etc.

In one aspect of the invention, is provided a fusion protein (epigenetic modification polypeptide) comprising from N-terminus to C-terminus, an epigenetic modification domain, an XTEN linker, and a nuclease-deficient RNA-guided DNA endonuclease enzyme or a nuclease-deficient endonuclease enzyme.

In aspects, the epigenetic modification polypeptide further comprises a transcriptional activator. In aspects, the transcriptional activator is VP64, p65, RTA, or a combination of two or more thereof. In another aspect, the epigenetic modification polypeptide further comprises one or more nuclear localization sequences. In embodiments, the epigenetic modification polypeptide comprises the nuclease-deficient RNA-guided DNA endonuclease enzyme. In embodiments, the fusion protein comprises the nuclease-deficient DNA endonuclease enzyme.

In some embodiments, the functional domains associated with the adaptor protein or the CRISPR enzyme is a transcriptional activation domain comprising VP64, p65, MyoD1, HSF1, RTA or SET7/9. Other references herein to activation (or activator) domains in respect of those associated with the adaptor protein(s) include any known transcriptional activation domain and specifically VP64, p65, MyoD1, HSF1, RTA or SET7/9 (see, e.g., U.S. Pat. No. 11,001,829B2).

In certain embodiments, the present invention provides a fusion protein comprising from N-terminus to C-terminus, an RNA-binding sequence, an XTEN linker, and a transcriptional activator. In aspects, the transcriptional activator is VP64, p65, RTA, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present invention provides a method of activating a target nucleic acid sequence in a cell, the method comprising: (i) delivering a first polynucleotide encoding a epigenetic modification polypeptide described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

### Transcription Repressors and Transcription Activators

In some embodiments, the method includes modulating gene expression of one or more target genes by modifying DNA binding sites and/or methylation sites for one or more DNA binding or interaction molecules or complexes. In some embodiments, the DNA binding or interaction molecules comprise, transcriptional activators, and/or transcriptional repressors. In some embodiments, the method comprises administering or otherwise introducing an engineered transcriptional activator or repressor to one or more cells such that expression of a target gene in any one is decreased or repressed or the expression of a target gene is increased or initiated.

### Exemplary Engineered Transcriptional Activators

In one example embodiment, a programmable nuclease is used to recruit an activator protein to a target gene in order to enhance expression. In another example embodiment, the programmable nuclease system is recruited to an enhancer possessing a variant. For example, a catalytically inactive Cas protein (“dCas”) fused to an activator can be used to recruit that activator protein to the mutated sequence. Accordingly, a guide sequence is designed to direct binding of the dCas-activator fusion such that the activator can interact with the target genomic region and induce expression of a gene. In one example embodiment, the guide is designed to bind within 10, 20, 30, 40, 50, 60, 70, 80, 90, 100 or up to 500 base pairs of the variant nucleotide. In one example embodiment, a CRISPR guide sequence includes the specific variant nucleotide. The Cas protein used may be any of the Cas proteins described elsewhere herein. In one example protein, the Cas protein is a dCas9.

In one embodiment, the programmable nuclease system is a CRISPRa system (see, e.g., US20180057810A1; and Konermann et al. “Genome-scale transcriptional activation by an engineered CRISPR-Cas9 complex” Nature. 2014 Dec. 10. doi: 10.1038/nature14136). Numerous genetic variants associated with disease phenotypes are found to be in non-coding region of the genome, and frequently coincide with transcription factor (TF) binding sites and non-coding RNA genes. In one embodiment, a CRISPR system may be used to activate gene transcription. A nuclease-dead RNA-guided DNA binding domain, dCas9, tethered to transcriptional activator domains that promote gene activation (e.g., p65) may be used for “CRISPRa” that activates transcription. In one example embodiment, for use of dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, one or more activator domains are recruited. In one example embodiment, the activation domain is linked to the CRISPR enzyme. In another example embodiment, the guide sequence includes aptamer sequences that bind to adaptor proteins fused to an activation domain. In general, the positioning of the one or more activator domains on the inactivated CRISPR enzyme or CRISPR complex is one which allows for correct spatial orientation for the activator domain to affect the target with the attributed functional effect. For example, the transcription activator is placed in a spatial orientation which allows it to affect the transcription of the target. This may include positions other than the N-/C-terminus of the CRISPR enzyme.

Other programmable nucleases can be modified similarly to provide transcriptional activation. In another example embodiment, an OMEGA system is used to recruit an activation domain to a gene. In one example embodiment, the activation domain is linked to the OMEGA protein. In general, the positioning of the one or more activator domains on the OMEGA protein is one which allows for correct spatial orientation for the activator domain to affect the target with the attributed functional effect. Similar to CRISPRa, the recruitment of the activation domain can increase expression of a gene.

In another example embodiment, a zinc finger system is used to recruit an activation domain to a gene. In one example embodiment, the activation domain is linked to the zinc finger system. In general, the positioning of the one or more activator domains on the zinc finger system is one which allows for correct spatial orientation for the activator domain to affect the target with the attributed functional effect. Similar to CRISPRa, the recruitment of the activation domain can increase expression of the gene.

In another example embodiment, a TALE system is used to recruit an activation domain to of a gene. In one example embodiment, the activation domain is linked to the TALE system. In general, the positioning of the one or more activator domains on the TALE system is one which allows for correct spatial orientation for the activator domain to affect the target with the attributed functional effect. For example, the transcription activator is placed in a spatial orientation which allows it to affect the transcription of the target. Similar to CRISPRa, the recruitment of the activation domain can increase expression of the gene.

In another example embodiment, a meganuclease system is used to recruit an activation domain to of a gene. In one example embodiment, the activation domain is linked to the meganuclease system. In general, the positioning of the one or more activator domains on the inactivated meganuclease system is one which allows for correct spatial orientation for the activator domain to affect the target with the attributed functional effect. For example, the transcription activator is placed in a spatial orientation which allows it to affect the transcription of the target. Similar to CRISPRa, the recruitment of the activation domain can increase expression of the gene.

### Exemplary Engineered Transcriptional Repressors

CRISPR interference (CRISPRi) is a CRISPR-Cas system variant that allows selective silencing or repression of gene expression by sterically repressing transcription by blocking transcription initiation or elongation (see e.g., Li et al., Cell. 152 (5): 1173-1183 (2013) of a target gene that is targeted by the dCas component of the system. A CRISPRi system comprises a dCas (e.g., dCas9) fused or otherwise linked to a repressor protein or domain (e.g., a KRAB (Kruppel-associated box) domain, mSin3A, NCoR (nuclear receptor co-repressor, Lsd1 (lysine-specific demethylase 1), MeCP2 (methyl-CpG-binding protein 2, HP1 (heterochromatin protein 1), and REST (RE1-silencing transcription factor)). In operation, the dCas portion is directed to a target gene whose expression is to be repressed, by a target gene specific guide RNA. The repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some exemplary embodiments, the CRISPRi system is configured to target one or more regions of a gene. In some exemplary embodiments, the CRISPRi system is configured to target one or more regions of a promoter or other regulatory region a gene.

In certain embodiments, the invention provides for a method of blocking transcription of one or more genes, comprising (i) a CRISPR guide targeting a genomic sequence encoding one or more of the genes and a modified Cas protein that is catalytically inactive, wherein the CRISPR guides optionally comprise a loop capable of binding a transcriptional activator domain or a transcription repressor domain. In certain embodiments, the modified Cas protein is optionally linked to a transcription activator domain or a transcription repressor domain.

In certain embodiments, the modified Cas protein is Cas9, Cpf1, C2c1, or C2c3. The modified Cas protein can be fused to a transcription activator domain or a transcription repressor domain. Alternatively, the CRISPR guides comprise a loop capable of binding a transcriptional activator domain or a transcription repressor domain.

Other programmable nucleases can be modified similarly to provide transcriptional repression. In some embodiments, an OMEGA is used to sterically inhibit transcription of one or more genes. In one example embodiment, the OMEGA protein is fused or otherwise linked to a repressor protein or domain (e.g., a KRAB (Kruppel-associated box) domain, mSin3A, NCoR (nuclear receptor co-repressor, Lsd1 (lysine-specific demethylase 1), MeCP2 (methyl-CpG-binding protein 2, HP1 (heterochromatin protein 1), and REST (RE1-silencing transcription factor)). In operation, the OMEGA protein directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some embodiments, a TALEN system is used to sterically inhibit transcription of one or more genes. In one example embodiment, the TALEN is fused or otherwise linked to a repressor protein or domain (e.g., a KRAB (Kruppel-associated box) domain, mSin3A, NCoR (nuclear receptor co-repressor, Lsd1 (lysine-specific demethylase 1), MeCP2 (methyl-CpG-binding protein 2, HP1 (heterochromatin protein 1), and REST (RE1-silencing transcription factor)). In operation, the TALEN directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some embodiments, a zinc finger system is used to sterically inhibit transcription of one or more genes. In one example embodiment, the ZFN is fused or otherwise linked to a repressor protein or domain (e.g., a KRAB (Kruppel-associated box) domain, mSin3A, NCoR (nuclear receptor co-repressor, Lsd1 (lysine-specific demethylase 1), MeCP2 (methyl-CpG-binding protein 2, HP1 (heterochromatin protein 1), and REST (RE1-silencing transcription factor)). In operation, the ZFN directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some embodiments, a Meganuclease system is used to sterically inhibit transcription of one or more genes. In one example embodiment, the Meganuclease is fused or otherwise linked to a repressor protein or domain (e.g., a KRAB (Kruppel-associated box) domain, mSin3A, NCoR (nuclear receptor co-repressor, Lsd1 (lysine-specific demethylase 1), MeCP2 (methyl-CpG-binding protein 2, HP1 (heterochromatin protein 1), and REST (RE1-silencing transcription factor)). In operation, the Meganuclease directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

### CTCF Editing

In one example embodiment, the perturbation comprises administering a CTCF-based gene modification system. In one example embodiment, the CTCF-based gene modification system configured to modify the one or more target genes is a genetic modification system capable of editing one or more CTCF motifs in the cell genome. In an example embodiment, editing one or more CTCF motifs modifies one or more chromosome loops such that expression of one or more target genes and transcription factors is decreased and/or is increased.

As used herein, the terms “CTCF” refers to the architectural protein CCCTC-binding factor. As used herein, the term “CTCF binding motif” refers a consensus DNA sequence (typically, 5′-CCACNAGGTGGCAG-′3 (SEQ ID NO: 32)) to which CTCF and two other proteins of the chromosomal loop forming complex, SMC3 and RAD21 bind. A loop domain is defined between two convergent pairs of CTCF-binding motifs. A chromosome loop refers to the genomic sequences in close proximity to each other (in any degree) that lie on the same chromosome (configured in cis), and also includes the architectural machinery involved in maintaining them (e.g., proteins, non-coding RNAs, DNA regulatory elements, etc.). The CTCF or the chromosome loop is maintained by architectural or DNA machinery associated with the CTCF or the chromosome loop and can facilitate interactions between remote enhancers and their target gene promoters to modulate transcription. (See, e.g., Guo et al. (2018).

Accordingly, gene expression can be modified by modification of where chromosomal loops are formed. Chromosomal loops can be removed, e.g., by removing or disrupting one or both of the CTCF motifs, or introduced by creation of a convergent pair of CTCF motifs, either by adding one CTCF motif that is in convergent orientation to a pre-existing CTCF motif or by insertion of a convergent CTCF pair. Any programmable nuclease (CRISPR-Cas, OMEGA systems, Zn Finger Nucleases, TALENs, meganucleases, base editors, prime editors, CAST systems, non-LTR retrotransposons) may be configured to insert, disrupt, or remove CTCF motifs. Three-dimensional chromosome mapping techniques such as Hi-C may be used to determine where loop boundaries are found and accordingly which CTCF motifs should be targeted to remove a loop and silence gene expression or introduce a loop and promote increased gene expression. See, e.g., Rao et al. “A 3D Map of the Human Genome at Kilobase Resolution Reveals Principles of Chromatin Looping” Cell, 159(7): 1665-1680 (2014).

In one embodiment, the system is configured to make a substitution, deletion or insertion of a CTCF motif or chromosome loop such that an enhancer and/or target gene promoter that regulates transcription of one or more target genes does not initiate transcription of the target gene, thereby reducing the expression of the one or more target genes or transcription factors.

In certain embodiments, the substitution, deletion, or insertion of a CTCF motif or chromosome loop initiates transcription of one or more target genes or transcription factors such that one or more genes or transcription factors are increased.

### RNAi and Antisense Oligonucleotides (ASO)

In one example embodiment, a perturbation comprises one or more RNAi agents directed to one or more genes such that expression of the one or more genes is reduced. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%. Additionally, inhibitory nucleic acid molecules such as RNAi and ASOs can be used in vivo (see, e.g., Yan Y, Liu X Y, Lu A, Wang X Y, Jiang L X, Wang J C. Non-viral vectors for RNA delivery. J Control Release. 2022; 342:241-279).

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

Antisense therapy is a form of treatment that uses antisense oligonucleotides (ASOs) to target messenger RNA (mRNA). ASOs are capable of altering mRNA expression through a variety of mechanisms, including ribonuclease H mediated decay of the pre-mRNA, direct steric blockage, and exon content modulation through splicing site binding on pre-mRNA (see, e.g., Crooke S T, Liang X H, Baker B F, Crooke R M. Antisense technology: A review. J Biol Chem. 2021; 296:100416. doi:10.1016/j.jbc.2021.100416). Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Commonly used antisense mechanisms to degrade target RNAs include RNase H1-dependent and RISC-dependent mechanisms. Preferred ASOs include Locked Nucleic Acid (LNA), Peptide Nucleic Acid (PNA), and morpholinos.

Combination with Other Optical Assays

In example embodiments, the methods described herein can be further combined to any additional phenotypes detectable by microscopy. In example embodiments, the additional phenotypes comprise cell morphology or biomolecule organization, including those detected by live cell markers, immunostaining, histological staining, or other similar methods. In example embodiments, the one or more additional phenotypes comprise any time resolved phenotype, such as, ion indicators, (e.g., calcium, sodium, magnesium, zinc, pH, and membrane potential indicators), voltage imaging, dynamic metabolite measurements, markers of cell stress, and/or cell migration. In example embodiments, a movie is taken of the plurality of cells after perturbation and before fixing. In example embodiments, live cell imaging is performed.

### Kits

In an aspect, the invention provides kits containing any one or more of the elements discussed herein. For example, a kit may include any embodiment of perturbation constructs, including a library of perturbation constructs capable of perturbing a plurality of gene targets. For example, a kit may include any embodiment of encoding probes, anchor probes, and readout probes. Additionally, kits may include phage DNA dependent RNA polymerase.

Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular process, or in a form that requires addition of one or more other components before use (e.g., in concentrate or lyophilized form).

Further embodiments are illustrated in the following Examples which are given for illustrative purposes only and are not intended to limit the scope of the invention.

### EXAMPLES

### Example 1—Perturb-FISH Provides for an all-Optical Genetic Screen of Intracellular and Intercellular Transcriptional Circuits

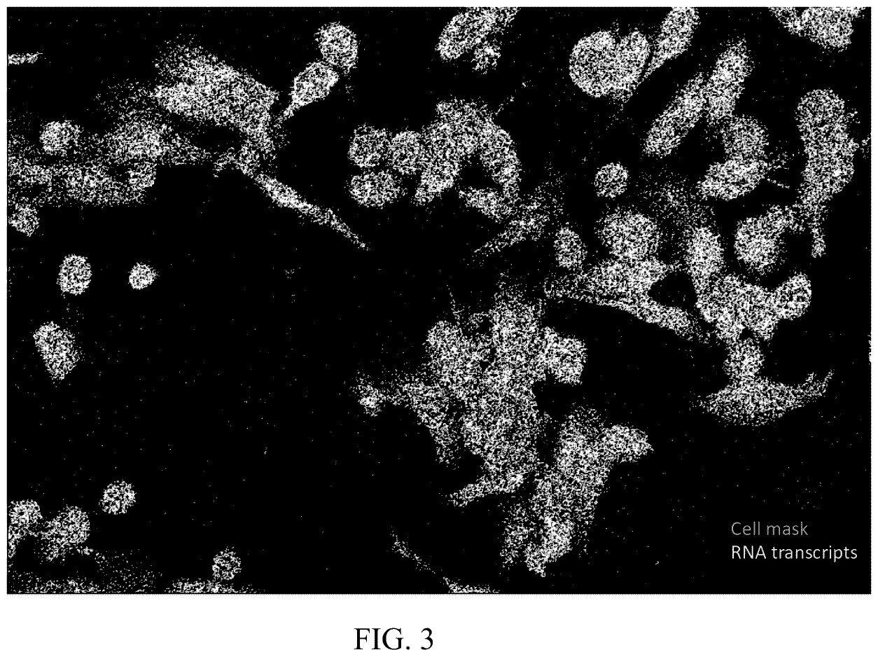

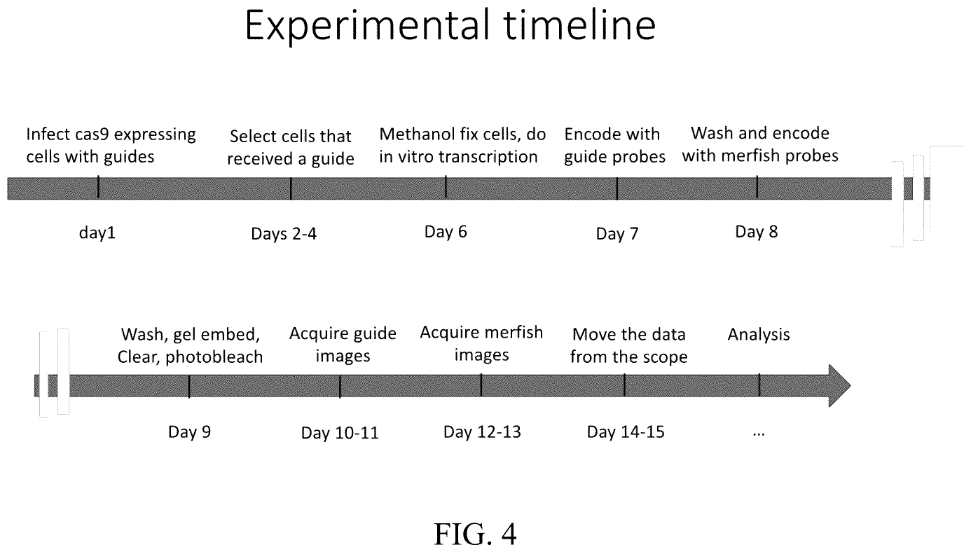

Perturb-FISH uses in situ transcription from a phage promoter in fixed cells to amplify guide RNA sequences from a perturbation construct that can be optically identified using guide RNA specific encoding probes (FIG. 1). Each encoding probe includes a targeting sequence capable of hybridizing to the guide RNA sequence and four readout sequences that make up a guide RNA barcode. Readout probes identify each readout sequence. FIG. 2A shows that RNA identity is encoded across 15 images. Each gRNA used in the experiment is expected to be “on” in 4 out of 15 images (i.e., 4 readout sequences). Each barcode is different from any other barcode by at least 4 bits (hamming weight 4 hamming distance 4, as was used in MERFISH), allowing error correction (i.e., if a spot appears “on” 5 times, it can be corrected and the identity decoded). FIG. 2B Shows a single image for a field of cells. Applicants combined detection of the perturbation identity with mRNA transcripts identified with MERFISH (FIG. 3). Applicants provide an exemplary timeline for a perturb-FISH experiment (FIG. 4).

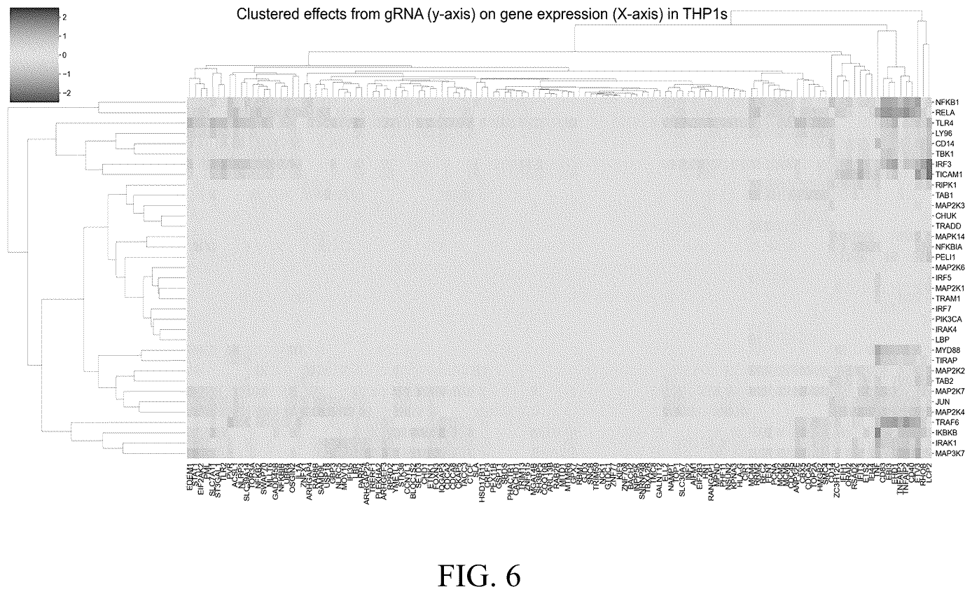

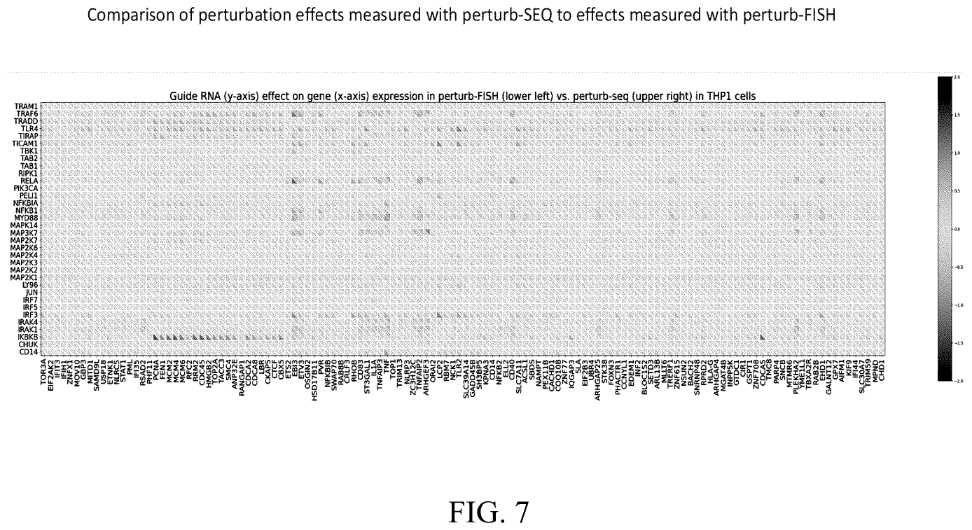

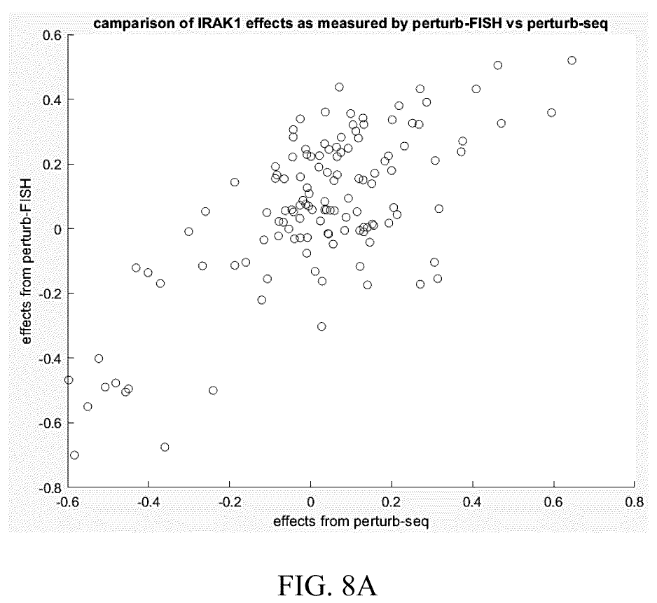

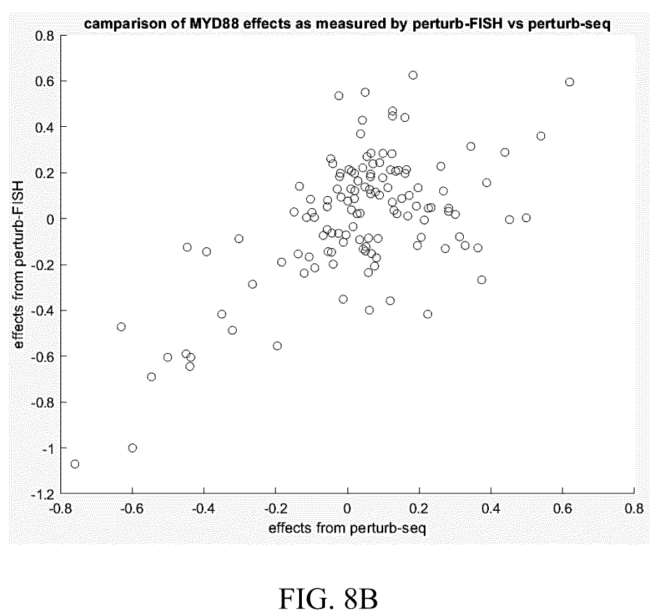

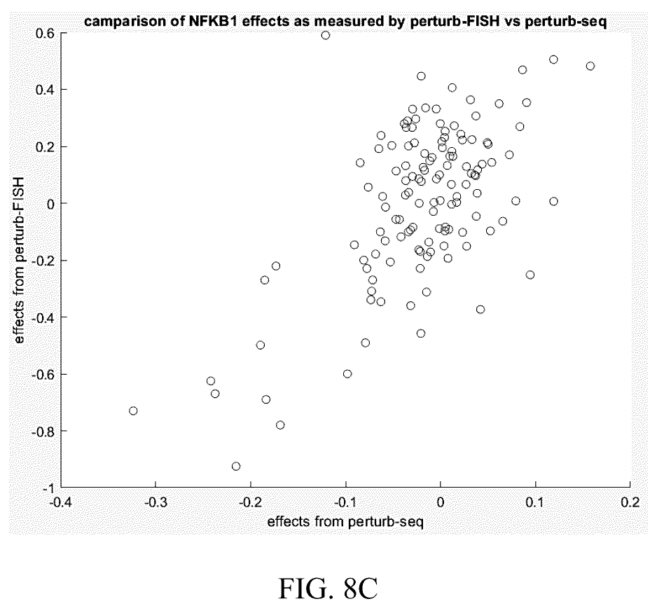

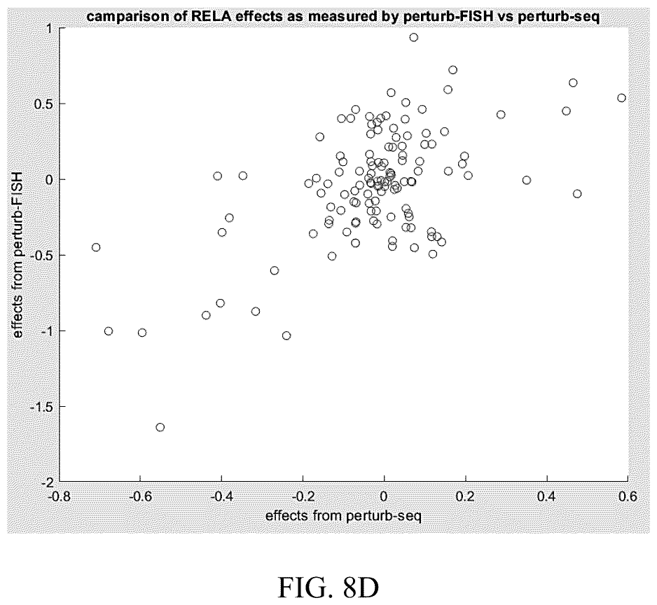

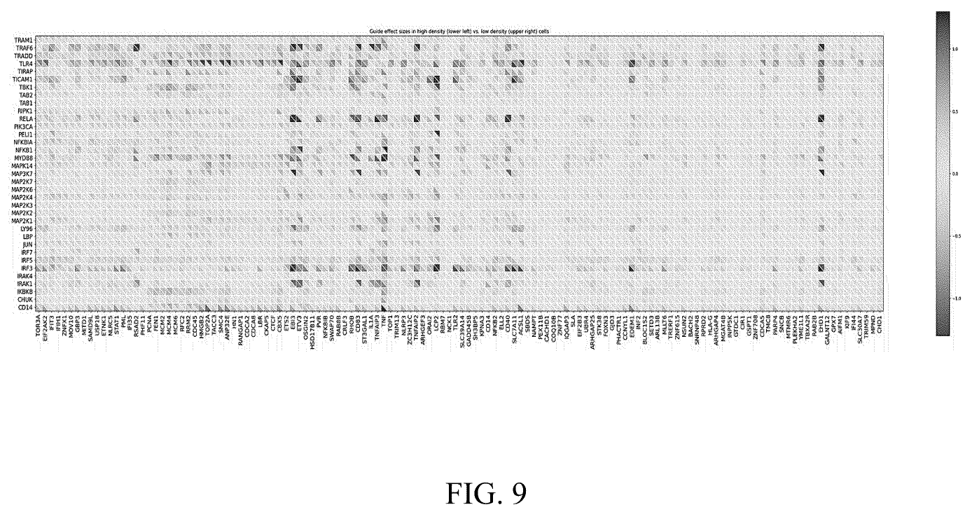

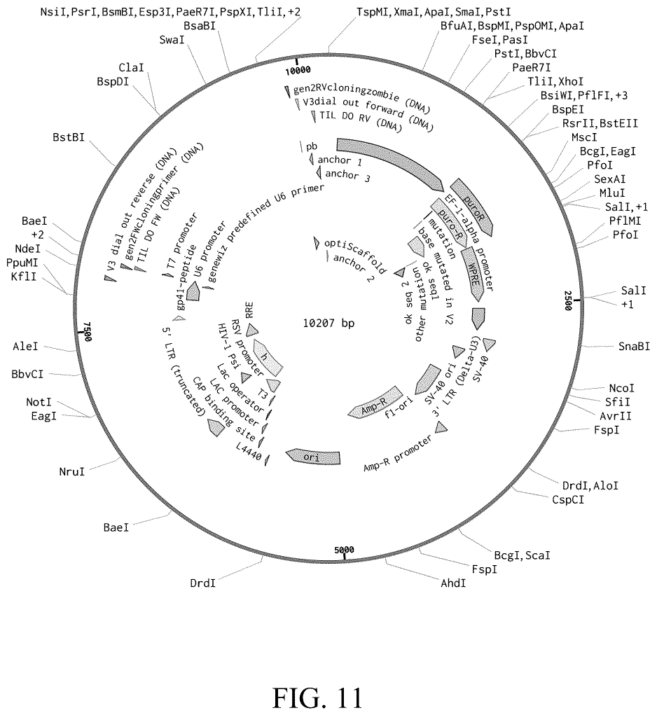

Applicants show the distributions of guides during a perturb-FISH experiment (FIG. 5). The distributions between cloned gRNAs and decoded gRNAs are expected to closely match Variation comes from errors in the protocol, but also in differences in cell proliferation, toxicity of some guides, and stochasticity of infections. Applicants performed multiplexed perturbations in THP1 cells and identified changes in gene expression associated with each perturbation (FIG. 6). Applicants used normal perturb-seq data as the “ground truth” and compared to perturb-FISH effects and found very high correlation (FIG. 7). Applicants found high correlation between some genes and lower correlation between other genes when comparing perturb-seq and perturb-FISH for four perturbed genes (FIG. 8). Applicants observed variations in gene expression after perturbation when comparing cell density (FIG. 9). FIG. 10 shows each step of an exemplary perturb-FISH experiment to identify gene networks in the macrophage response to LPS stimulation. Applicants used a lentiviral vector to generate lentivirus for each perturbation. The perturb-FISH vector is described in FIG. 10A, and the map and full sequence of the vector is shown in FIGS. 11 and 12.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method for perturbation screening with spatially resolved readouts comprising:
a) perturbing a plurality of cells by introducing one or more perturbation constructs to the plurality of cells, each perturbation construct encoding for one or more sequence specific perturbations, wherein the one or more sequence specific perturbations comprise a perturbation sequence identifying the perturbation, said sequence operably linked to at least two promoters comprising a Pol III promoter and a phage promoter, and wherein the plurality of cells maintains a spatial localization;
b) fixing the perturbed cells, whereby the spatial localization of the plurality of cells is fixed;
c) contacting the fixed cells with one or more phage polymerases and reagents for in vitro transcription of the perturbation sequences;
d) encoding the plurality of cells by contacting the plurality of cells with:
i) encoding probes specific for a perturbation sequence, each probe comprising a targeting sequence specific for one perturbation sequence and one or more readout sequences specific for each perturbation and
ii) encoding probes specific for mRNAs expressed in the plurality of cells, each probe comprising a targeting sequence specific for an mRNA sequence and one or more readout sequences specific for each mRNA sequence;
e) contacting the fixed and encoded plurality of cells with fluorescently labeled readout probes specific for a readout sequence and acquiring spatially resolved images for each readout probe using a microscope; and
f) decoding the perturbation and mRNA expression for each cell in the plurality of cells based on the images acquired.

2. The method of claim 1, wherein the one or more perturbation constructs comprise one or more anchor sequences downstream of the perturbation sequence, wherein the one or more anchor sequences are the same for every perturbation construct.

3. The method of claim 2, wherein the encoding step (d) further comprises contacting the plurality of cells with acrydite-modified anchor probes comprising a sequence specific to an anchor sequence and contacting the plurality of cells with acrydite-modified anchor probes comprising a poly(dT) sequence, wherein the method further comprises embedding the fixed cells in a polymerized hydrogel, and removing cell components not linked to the hydrogel through an anchor probe.

4. The method of claim 3, wherein the polymerized hydrogel is a non-swellable hydrogel, a swellable hydrogel, a polyacrylamide hydrogel, or a polyacrylate hydrogel.

5. (canceled)

6. (canceled)

7. The method of claim 1, wherein the plurality of cells is grown on a solid support to maintain the spatial localization.

8. (canceled)

9. (canceled)

10. (canceled)

11. The method of claim 1, wherein the one or more sequence specific perturbations is made using a CRISPR system and the perturbation sequence is a guide sequence.

12. (canceled)

13. The method of claim 1, wherein the one or more sequence specific perturbations is made using an RNAi or antisense system, and the perturbation sequence is an RNAi or antisense sequence.

14. (canceled)

15. The method of claim 1, wherein the readout sequences of the encoding probes for encoding the perturbation sequences are different from the readout sequences of the encoding probes for encoding the mRNA sequences, and wherein step (f) is performed in two steps, one step for perturbations and one step for mRNA.

16. The method of claim 1, wherein the one or more perturbation constructs are integrated into a genome of the perturbed cells.

17. The method of claim 1, wherein the one or more perturbation constructs are introduced by a viral vector at a multiplicity of infection (MOI) where each cell in the plurality of cells receives one or more zero perturbation constructs.

18. (canceled)

19. (canceled)

20. (canceled)

21. The method of claim 1, further comprising linking the perturbation and mRNA expression to one or more additional phenotypes detectable by microscopy.

22. The method of claim 21, wherein the one or more additional phenotypes comprise a cell morphology, biomolecule organization, or time resolved phenotype.

23. (canceled)

24. The method of claim 1, wherein after step (a), perturbation, and before step (b), fixing, a movie of the plurality of cells is recorded, wherein the movie records live cell markers.

25. (canceled)

26. A kit comprising a library of perturbation constructs, each perturbation construct encoding for one or more sequence specific perturbations, wherein the one or more sequence specific perturbations comprise a perturbation sequence identifying the perturbation, said sequence operably linked to at least two promoters comprising a Pol III promoter and a phage promoter.

27. The kit of claim 26, wherein the one or more sequence specific perturbations comprises a CRISPR system, wherein the perturbation sequence is a guide sequence.

28. (canceled)

29. (canceled)

30. The kit of claim 26, further comprising encoding probes specific for a perturbation sequence, each probe comprising a targeting sequence specific for one perturbation sequence and one or more readout sequences specific for each perturbation.

31. The kit of claim 26, further comprising encoding probes specific for mRNA sequences, each probe comprising a targeting sequence specific for an mRNA sequence and one or more readout sequences specific for each mRNA sequence.

32. The kit of claim 26, wherein the perturbation constructs comprise one or more anchor sequences downstream of the perturbation sequence, wherein the one or more anchor sequences are the same for every perturbation construct.

33. The kit of claim 32, further comprising acrydite-modified anchor probes comprising a sequence specific to the one or more anchor sequences downstream of the perturbation sequence.

34. (canceled)

35. (canceled)

36. (canceled)

37. A method of identifying gene regulatory interactions in a spatially organized population of cells, comprising:
a) introducing a library of perturbation constructs into a plurality of cells maintaining spatial localization, each perturbation construct encoding a sequence-specific perturbation identified by a perturbation sequence;
b) fixing the spatially organized plurality of cells;
c) simultaneously determining, for each cell, the identify of the perturbation and the transcriptome of that cell using spatially resolved nucleic acid imaging; and
d) correlating perturbation identity with transcriptional changes across the population of cells to identify gene regulatory interactions.


SEQUENCE LISTING (32 sequences)

SEQ ID NO: 1 - DNA, 10207 bp, synthetic construct
  misc_feature 1..10207: note = Synthetic
  source 1..10207: mol_type = other DNA
cccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgcccgctagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgctaatacgactcactataggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgtttaagagctatgctggaaacagcatagcaagtttaaataaggctagtccgttatcaacttgaaaaagtggcaccgagtcggtgcttttttaatcgcttcatgcaaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggggg

SEQ ID NO: 2 - DNA, 10207 bp, synthetic construct
  misc_feature 1..10207: note = Synthetic
  source 1..10207: mol_type = other DNA
gggcccacgtttctacctatttcaaaatttgtctctccttagaaacgtcgattacctggaagatccagaactttcctcacccttaaccgaggccacgggcagtcacccgtctcgcgtgtagcgggtgtcaggggctcttcaacccccctccccagccgttaactaggccacggatctcttccaccgcgccccatttgaccctttcactacagcacatgaccgaggcggaaaaagggctcccaccccctcttggcatatattcacgtcatcagcggcacttgcaagaaaaagcgttgcccaaacggcggtcttgtgtccattcacggcacacaccaagggcgcccggaccggagaaatgcccaataccgggaacgcacggaacttaatgaaggtggaccgacgtcatgcactaagaactagggctcgaagcccaaccttcacccaccctctcaagctccggaacgcgaattcctcggggaagcggagcacgaactcaactccggaccggacccgcgaccccggcggcgcacgcttagaccaccgtggaagcgcggacagagcgacgaaagctattcagagatcggtaaattttaaaaactactggacgacgctgcgaaaaaaagaccgttctatcagaacatttacgcccggttctagacgtgtgaccataaagccaaaaaccccggcgcccgccgctgccccgggcacgcagggtcgcgtgtacaagccgctccgccccggacgctcgcgccggtggctcttagcctgcccccatcagagttcgaccggccggacgagaccacggaccggagcgcggcggcacatagcggggcgggacccgccgttccgaccgggccagccgtggtcaacgcactcgcctttctaccggcgaagggccgggacgacgtccctcgagttttacctcctgcgccgcgagccctctcgcccgcccactcagtgggtgtgtttccttttcccggaaaggcaggagtcggcagcgaagtacactgaggtgcctcatggcccgcggcaggtccgtggagctaatcaagagctcgaaaacctcatgcagcagaaatccaacccccctccccaaaatacgctacctcaaaggggtgtgactcacccacctctgacttcaatccggtcgaaccgtgaactacattaagaggaaccttaaacgggaaaaactcaaacctagaaccaagtaagagttcggagtctgtcaccaagtttcaaaaaaagaaggtaaagtccacagcactgcatgccggtggtactggctcatgttcgggtgccacgcggagcggtgggcgctgctgcaggggtcccggcatgcgtgggagcggcggcgcaagcggctgatggggcggtgcgcggtgtggcagctaggcctggcggtgtagctcgcccagtggctcgacgttcttgagaaggagtgcgcgcagcccgagctgtagccgttccacacccagcgcctgctgccgcggcggcaccgccagacctggtgcggcctctcgcagcttcgcccccgccacaagcggctctagccgggcgcgtaccggctcaactcgccaagggccgaccggcgcgtcgttgtctaccttccggaggaccgcggcgtggccgggttcctcgggcgcaccaaggaccggtggcagcctcagagcgggctggtggtcccgttcccagacccgtcgcggcagcacgaggggcctcacctccgccggctcgcgcggccccacgggcggaaggacctctggaggcgcggggcgttggaggggaagatgctcgccgagccgaagtggcagtggcggctgcagctccacgggcttcctggcgcgtggaccacgtactgggcgttcgggccacggacttgcgcaattcagctgttagttggagacctaatgttttaaacactttctaactgaccataagaattgatacaacgaggaaaatgcgatacacctatgcgacgaaattacggaaacatagtacgataacgaagggcataccgaaagtaaaagaggaggaacatatttaggaccaacgacagagaaatactcctcaacaccgggcaacagtccgttgcaccgcaccacacgtgacacaaacgactgcgttgggggtgaccaaccccgtaacggtggtggacagtcgaggaaaggccctgaaagcgaaagggggagggataacggtgccgccttgagtagcggcggacggaacgggcgacgacctgtccccgagccgacaacccgtgactgttaaggcaccacaacagcccctttagtagcaggaaaggaaccgacgagcggacacaacggtggacctaagacgcgccctgcaggaagacgatgcagggaagccgggagttaggtcgcctggaaggaagggcgccggacgacggccgagacgccggagaaggcgcagaagcggaagcgggagtctgctcagcctagagggaaacccggcggaggggcgcagctgaaattctggttactgaatgttccgtcgacatctagaatcggtgaaaaattttcttttcccccctgaccttcccgattaagtgagggttgcttctgttctagacgaaaaacgaacatgacccagagagaccaatctggtctagactcggaccctcgagagaccgattgatcccttgggtgacgaattcggagttatttcgaacggaactcacgaagttcatcacacacgggcagacaacacactgagaccattgatctctagggagtctgggaaaatcagtcacaccttttagagatcgtcatgcatatcatcaagtacagtagaataataagtcataaatattgaacgtttctttacttatagtctctcactctccttgaacaaataacgtcgaatattaccaatgtttatttcgttatcgtagtgtttaaagtgtttatttcgtaaaaaaagtgacgtaagatcaacaccaaacaggtttgagtagttacatagaatagtacagaccgagatcgatagggcggggattgaggcgggtagggcggggattgaggcgggtcaaggcgggtaagaggcggggtaccgactgattaaaaaaaataaatacgtctccggctccggcggagccggagactcgataaggtcttcatcactcctccgaaaaaacctccggatccctgcatgggttaagcgggcgatcactcagcataatgcgcgcgagtgaccggcagcaaaatgttgcagcactgacccttttgggaccgcaatgggttgaattagcggaacgtcgtgtagggggaaagcggtcgaccgcattatcgcttctccgggcgtggctagcgggaagggttgtcaacgcgtcggacttaccgcttaccctgcgcgggacatcgccgcgtaattcgcgccgcccacaccaccaatgcgcgtcgcactggcgatgtgaacggtcgcgggatcgcgggcgaggaaagcgaaagaagggaaggaaagagcggtgcaagcggccgaaaggggcagttcgagatttagcccccgagggaaatcccaaggctaaatcacgaaatgccgtggagctggggttttttgaactaatcccactaccaagtgcatcacccggtagcgggactatctgccaaaaagcgggaaactgcaacctcaggtgcaagaaattatcacctgagaacaaggtttgaccttgttgtgagttgggatagagccagataagaaaactaaatattccctaaaacggctaaagccggataaccaattttttactcgactaaattgtttttaaattgcgcttaaaattgttttataattgcgaatgttaaatccaccgtgaaaagcccctttacacgcgccttggggataaacaaataaaaagatttatgtaagtttatacataggcgagtactctgttattgggactatttacgaagttattataactttttccttctcatactcataagttgtaaaggcacagcgggaataagggaaaaaacgccgtaaaacggaaggacaaaaacgagtgggtctttgcgaccactttcattttctacgacttctagtcaacccacgtgctcacccaatgtagcttgacctagagttgtcgccattctaggaactctcaaaagcggggcttcttgcaaaaggttactactcgtgaaaatttcaagacgatacaccgcgccataatagggcataactgcggcccgttctcgttgagccagcggcgtatgtgataagagtcttactgaaccaactcatgagtggtcagtgtcttttcgtagaatgcctaccgtactgtcattctcttaatacgtcacgacggtattggtactcactattgtgacgccggttgaatgaagactgttgctagcctcctggcttcctcgattggcgaaaaaacgtgttgtaccccctagtacattgagcggaactagcaacccttggcctcgacttacttcggtatggtttgctgctcgcactgtggtgctacggacatcgttaccgttgttgcaacgcgtttgataattgaccgcttgatgaatgagatcgaagggccgttgttaattatctgacctacctccgcctatttcaacgtcctggtgaagacgcgagccgggaaggccgaccgaccaaataacgactatttagacctcggccactcgcacccagagcgccatagtaacgtcgtgaccccggtctaccattcgggagggcatagcatcaatagatgtgctgcccctcagtccgttgatacctacttgctttatctgtctagcgactctatccacggagtgactaattcgtaaccattgacagtctggttcaaatgagtatatatgaaatctaactaaattttgaagtaaaaattaaattttcctagatccacttctaggaaaaactattagagtactggttttagggaattgcactcaaaagcaaggtgactcgcagtctggggcatcttttctagtttcctagaagaactctaggaaaaaaagacgcgcattagacgacgaacgtttgtttttttggtggcgatggtcgccaccaaacaaacggcctagttctcgatggttgagaaaaaggcttccattgaccgaagtcgtctcgcgtctatggtttatgacaagaagatcacatcggcatcaatccggtggtgaagttcttgagacatcgtggcggatgtatggagcgagacgattaggacaatggtcaccgacgacggtcaccgctattcagcacagaatggcccaacctgagttctgctatcaatggcctattccgcgtcgccagcccgacttgccccccaagcacgtgtgtcgggtcgaacctcgcttgctggatgtggcttgactctatggatgtcgcactcgatactctttcgcggtgcgaagggcttccctctttccgcctgtccataggccattcgccgtcccagccttgtcctctcgcgtgctccctcgaaggtccccctttgcggaccatagaaatatcaggacagcccaaagcggtggagactgaactcgcagctaaaaacactacgagcagtccccccgcctcggatacctttttgcggtcgttgcgccggaaaaatgccaaggaccggaaaacgaccggaaaacgagtgtacaagaaaggacgcaataggggactaagacacctattggcataatggcggaaactcactcgactatggcgagcggcgtcggcttgctggctcgcgtcgctcagtcactcgctccttcgccttctcgcgggttatgcgtttggcggagaggggcgcgcaaccggctaagtaattacgtcgaccgtgctgtccaaagggctgacctttcgcccgtcactcgcgttgcgttaattacactcaatcgagtgagtaatccgtggggtccgaaatgtgaaatacgaaggccgagcatacaacacaccttaacactcgcctattgttaaagtgtgtcctttgtcgatactggtactaatgcggttcgcgcgttaattgggagtgatttcccttgttttcgacctcgacgttcgaattacatcagaatacgttatgagaacatcagaacgttgtaccattgctactcaatcgttgtacggaatgttcctctctttttcgtggcacgtacggctaaccaccttcattccaccatgctagcacggaataatccttccgttgtctgcccagactgtacctaacctgcttggtgacttaacggcgtaacgtctctataacataaattcacggatcgagctatgtatttgcccagagagaccaatctggtctagactcggaccctcgagagaccgattgatcccttgggtgacgaattcggagttatttcgaacggaactcacgaagttcatcacacacgggcagacaacacactgagaccattgatctctagggagtctgggaaaatcagtcacaccttttagagatcgtcaccgcgggcttgtccctgaactttcgctttccctttggtctcctcgagagagctgcgtcctgagccgaacgacttcgcgcgtgccgttctccgctccccgccgctgaccactcatgcggtttttaaaactgatcgcctccgatcttcctctctctacccacgctctcgcagtcataattcgccccctcttaatctagcgctacccttttttaagccaattccggtccccctttcttttttatatttaattttgtatatcatacccgttcgtccctcgatcttgctaagcgtcaattaggaccggacaatctttgtagtcttccgacatctgtttatgaccctgtcgatgttggtagggaagtctgtcctagtcttcttgaatctagtaatatattatgtcatcgttgggagataacacacgtagtttcctatctctattttctgtggttccttcgaaatctgttctatctccttctcgttttgttttcattctggtggcgtgtcgttcgccggcgactagaagtctggacctcctcctctatactccctgttaacctcttcacttaatatatttatatttcatcatttttaacttggtaatcctcatcgtgggtggttccgtttctcttctcaccacgtctctcttttttctcgtcacccttatcctcgaaacaaggaacccaagaaccctcgtcgtccttcgtgatacccgcgtcgcagttactgcgactgccatgtccggtctgttaataacagaccatatcacgtcgtcgtcttgttaaacgactcccgataactccgcgttgtcgtagacaacgttgagtgtcagaccccgtagttcgtcgaggtccgttcttaggaccgacacctttctatggatttcctagttgtcgaggacccctaaaccccaacgagaccttttgagtaaacgtggtgacgacacggaaccttacgatcaacctcattatttagagaccttgtctaaaccttagtgtgctggacctacctcaccctgtctctttaattgttaatgtgttcgaattatgtgaggaattaacttcttagcgttttggtcgttcttttcttacttgttcttaataaccttaatctatttacccgttcaaacaccttaaccaaattgtattgtttaaccgacaccatatattttaataagtattactatcatcctccgaaccatccaaattcttatcaaaaacgacatgaaagatatcacttatctcaatccgtccctataagtggtaatagcaaagtctgggtggagggttggggctcccctgggtctctcccggataaagggtactaaggaagtataaacgtatatgctatgttccgacaatctctctattaatcttaattaaactgacatttgtgtttctataatcatgttttatgcactgcatctttcattattaaagaacccatcaaacgtcaaaattttaatacaaaattttacctgatagtatacgaatggcattgaactttcataaagctaaagaaccgaaatatatagaacgattatgctgagtgatatcctctgccaacatttactcgtgtgttttatgtgtacgattttataatataagatactggaaatattttagttggttttagaagaaaaattattgaaatcatagttattaatcttaaaaatacaaggaaaaacgtttgaaaattatttttactcgttttatttttttgcgatcaaaatcattgagcgcaacaaaagaagtggaaattattatcgatgaggtggtgaacaaggattcgccagtcgaggacgaagttagtaaaaaactcgtagaagtttacaagattgaggtggtcgacgaaattgatttcgtaacagaaattgttgactgaagtaatcaaattgtagaagtttacaacgtggactaaaacttttaggacaactacaaaattgtttaagattaggtcgaagttgtcgataaagtgttcgaaagtactaaagaagaaaacaattatttgttaaaaggtattatgtaaattgttgtacactaggtcgacgaaaaaaatgtcgaaagtacagaagattttgattaagtattaaaaacagaaaattacgtggttataaattatggtatagttaaagacaacgtggtagaaattaacgaagtctttgaagcttacgaaaacatcgacaacacgtacgtggatctccttttggatgttgtaaacaataaggatgtaaacacggaaaattattaagaaatgttatcgaacaagttatacttaattgtgtttgacaacgttttagtttaagttaacgaagtagtgtattaacaaattaaagtcgaaagcatcgtagaacaaaattattacacactagatatataaacaaatcaaagtaaaaaagaggatatataagtaaaaattaaaattaagaaattattaaagcagatgaaattgaaatcgcaaaacttgtctaagtggttgtggatattttatttaaaaatcaaatccaagtcaaggtgaacccgcttgtcgtttagtactgaatagaagatttattttaaaatcattcagaacaggaccgtataatatgtaaggtagctacatcagaagttgtaattgttgaaattcaggtcgttaaactcaattcccacaacgagagttactaaagtaattaccaagttaaaaattaaagaaaagaagaccaaattttaagttcaaatttcactttcacattatacgtgggtaaagaaatttatttagaagatttatcagatgattacaaaataaaacaaaaaatattttagttcgtcggagacgataattatatcttcgaacataaggtagaaatagagatcgactcagtagttaatgtataggtattgaaagaagtattcgtttttgttttaaattaggcaatagaagaagaaatcgttaaagagatgggtaagtaaatttaggtcaatttcaaaaatgttataattgaggtataaaaagtactcgctaagatagtgggtttagtgaacaatgttttgaacttatatctcggcctaaaaaaccttacgataaattcgcaaaatctaaactattaaaagttagttaattttaaccaggacaaactaaaggtagattagaatgttttactggtagtacaaaataacggtaaggtttagacagtcgtagacccagtaagtattattattatagacgtagtacaaattatggtataagttcgccataaaaagtacgtcctagtttaagacctaaacctaaatgttgtaaaaatttacaaagtagaagtttacgtacgagaagttggagttattgcaatataggactaagtgcattataaaaaccccatttaaatcaaggacaaggtaattgacgcgatttttattaaaaatttagaaaaaatcgaagaacgagaaaaaacatgcagagacaaattctcgatacgacctttgtcgtatcgttcaaatttattccgatcaggcaatagttgaactttttcaccgtggctcagccacgaaaaaattagcgaagtacgtttcgaaccgcattgatctagaactctgtttaccgtcataagtaggtgttaaaattttcttttcccccctaaccccccatgtcacgtcccctttcttatcatctgtattatcgttgtctgtatgtttgatttcttaatgtttttgtttaatgtttttaagttttaaaagcccaaataatgtccctgtcgtctctaggtgaaaccgcggccgagctccccc

SEQ ID NO: 3 - protein, 198 aa, synthetic construct
  REGION 1..198: note = Synthetic
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPG

SEQ ID NO: 4 - DNA, 32 bp, synthetic construct
  source 1..32: mol_type = other DNA
  misc_feature 1..32: note = Synthetic
cgtgacgtagaaagtaataatttcttgggtag

SEQ ID NO: 5 - DNA, 27 bp, synthetic construct
  source 1..27: mol_type = other DNA
  misc_feature 1..27: note = Synthetic
tatcttgctaatacgactcactatagg

SEQ ID NO: 6 - DNA, 30 bp, synthetic construct
  source 1..30: mol_type = other DNA
  misc_feature 1..30: note = Synthetic
ttatatatcttgctaatacgactcactata

SEQ ID NO: 7 - DNA, 21 bp, synthetic construct
  misc_feature 1..21: note = Synthetic
  source 1..21: mol_type = other DNA
gatacgacctttgtcgtatcg

SEQ ID NO: 8 - DNA, 19 bp, synthetic construct
  misc_feature 1..19: note = Synthetic
  source 1..19: mol_type = other DNA
accgtggctcagccacgaa

SEQ ID NO: 9 - DNA, 25 bp, synthetic construct
  misc_feature 1..25: note = Synthetic
  source 1..25: mol_type = other DNA
tgtcacgtcccctttcttatcatct

SEQ ID NO: 10 - protein, 7 aa, unidentified
  source 1..7: note = Simian virus 40
PKKKRKV

SEQ ID NO: 11 - protein, 10 aa, unidentified
  source 1..10: note = Simian virus 40
PKKKRKVEAS

SEQ ID NO: 12 - protein, 16 aa, synthetic construct
  REGION 1..16: note = Synthetic
KRPAATKKAGQAKKKK

SEQ ID NO: 13 - protein, 9 aa, Homo sapiens
PAAKRVKLD

SEQ ID NO: 14 - protein, 11 aa, Homo sapiens
RQRRNELKRSP

SEQ ID NO: 15 - protein, 38 aa, Homo sapiens
NQSSNFGPMKGGNFGGRSSGPYGGGGQYFAKPRNQGGY

SEQ ID NO: 16 - protein, 42 aa, Homo sapiens
RMRIZFKNKGKDTAELRRRRVEVSVELRKAKKDEQILKRRNV

SEQ ID NO: 17 - protein, 8 aa, Homo sapiens
VSRKRPRP

SEQ ID NO: 18 - protein, 8 aa, Homo sapiens
PPKKARED

SEQ ID NO: 19 - protein, 8 aa, Homo sapiens
PQPKKKPL

SEQ ID NO: 20 - protein, 12 aa, Mus sp.
SALIKKKKKMAP

SEQ ID NO: 21 - protein, 5 aa, unidentified
  source 1..5: note = Influenza virus
DRLRR

SEQ ID NO: 22 - protein, 7 aa, unidentified
  source 1..7: note = Influenza virus
PKQKKRK

SEQ ID NO: 23 - protein, 10 aa, unidentified
  source 1..10: note = Hepatitus D virus
RKLKKKIKKL

SEQ ID NO: 24 - protein, 10 aa, Mus sp.
REKKKFLKRR

SEQ ID NO: 25 - protein, 20 aa, Homo sapiens
KRKGDEVDGVDEVAKKKSKK

SEQ ID NO: 26 - protein, 17 aa, Homo sapiens
RKCLQAGMNLEARKTKK

SEQ ID NO: 27 - protein, 288 aa, synthetic construct
  REGION 1..288: note = Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 28 - protein, 183 aa, synthetic construct
  REGION 1..183: note = Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 29 - DNA, 42 bp, synthetic construct
  source 1..42: mol_type = other DNA
  misc_feature 1..42: note = Synthetic
ggctcgttgggaggtcccgggttgaaatcccggacgagcccg

SEQ ID NO: 30 - DNA, 100 bp, Homo sapiens
  source 1..100: mol_type = other DNA
tagccaaatgcctcgtcatctaattagtgacgcgcatgaatggatgaacgagattcccactgtccctacctactatccagcgaaaccacagccaagggaa

SEQ ID NO: 31 - DNA, 125 bp, Bombyx mori
  source 1..125: mol_type = other DNA
tagccaaatgcctcgtcatctaattagtgacgcgcatgaatggattaacgagattcccactgtccctatctactatctagcgaaaccacagccaagggaacgggcttgggagaatcagcggggaa

SEQ ID NO: 32 - DNA, 14 bp, synthetic construct
  source 1..14: mol_type = other DNA
  misc_feature 1..14: note = Synthetic
  variation 5: note = n is a, c, g, or t
ccacnaggtggcag